(19) United States
(12) Patent Application Publication
(10) Pub. No.: US 2021/0196822 A1
(43) Pub. Date: Jul. 1, 2021

(54) TREATMENT OF TRIPLE NEGATIVE BREAST CANCER WITH TARGETED TGF-B INHIBITION

(71) Applicant: Merck Patent GmbH, Darmstadt (DE)

(72) Inventors: George Locke, Arlington, MA (US); Isabelle Dussault, Needham, MA (US)

(21) Appl. No.: 17/176,427

(22) Filed: Feb. 16, 2021

Related U.S. Application Data
(63) Continuation of application No. PCT/US2019/047734, filed on Aug. 22, 2019.
(60) Provisional application No. 62/721,249, filed on Aug. 22, 2018.

(51) Int. Cl. A61K 39/395; A61K 38/17; A61K 9/00; A61P 35/00; A61M 5/158; A61M 5/178

(52) U.S. Cl. CPC A61K 39/3955; A61K 38/179; A61K 9/0019; A61K 2039/54; A61M 5/158; A61M 5/178; A61P 35/00

(57) ABSTRACT

The present disclosure relates generally to methods for treating a patient diagnosed with triple negative breast cancer (TNBC), involving identifying a patient likely to respond to treatment via targeted TGF-β inhibition with an anti-TGFβ agent, and treating the subject with the anti-TGFβ agent.

### CROSS-REFERENCE TO RELATED APPLICATIONS

This application claims the benefit of and priority to U.S. Provisional Patent Application No. 62/721,249, filed Aug. 22, 2018, the entire disclosures of which are incorporated by reference herein.

### SEQUENCE LISTING

The instant application contains a Sequence Listing which has been submitted electronically in ASCII format and is hereby incorporated by reference in its entirety. Said ASCII copy, created on Jun. 27, 2019, is named EMD-009WO_SL_ST25.txt and is 99,098 bytes in size.

### FIELD OF THE DISCLOSURE

The present disclosure relates generally to methods for treating a subject diagnosed with triple negative breast cancer (TNBC), involving identifying a subject likely to respond to treatment via targeted TGF-β inhibition with an anti-TGFβ agent, and treating the subject with the anti-TGFβ agent.

### BACKGROUND

TNBC is a heterogeneous group of breast cancer tumors that is usually diagnosed via immunohistochemistry for tumors that do not express estrogen receptor (ER) and progesterone receptor (PR) at all, and do not overexpress hormone epidermal growth factor receptor 2 (HER-2). TNBC is an aggressive type of cancer that is associated with a poor prognosis. Since the tumor cells lack the necessary receptors, common treatments like hormone therapy and drugs that target estrogen, progesterone, and HER-2 are ineffective. Doxorubicin is a standard-of-care, DNA-damaging agent used in the treatment of a host of malignancies, including locally advanced and recurrent or metastatic TNBC; the response to doxorubicin is dismal compared to other types of breast cancer.

Recent efforts to improve therapy for TNBC have focused on classifying the TNBC tumor type according to 4 (Lehmann, J. Clin. Invest. (2011) 121:2750-2767) or 6 (Burstein, Clin. Canc. Res. (2014) 21:1688-1698) sub-types: BL1 (basal-like 1), BL2 (basal-like 2), LAR (luminal androgen receptor), M (mesenchymal), IM (immune-modulatory) and MSL (mesenchymal-stem like) based on gene expression profiles. One promising approach is using PARP inhibitors in patients with BRCA1/2 mutations, a group of patients that constitutes 10-20% of TNBC patients. This sub-group of patients falls into the BL1 sub-type for which other therapies have also been suggested, such as CDK inhibitors and taxanes. The BL2 sub-type (22% of TNBC) includes genes enriched for growth factor signaling, which suggests that growth factor inhibitors (including kinase inhibitors) are potential therapies for this group. The UAR sub-types, as its name implies, contains the androgen receptor gene and therefore, anti-androgens are potential therapies in the group. Drugs targeting mesenchymal pathways such as c-Met inhibitors, TGFβ inhibitors and Wnt inhibitors have been proposed as potential therapies for the M sub-type. Finally, checkpoint inhibitors (CPI) may be good choices for the IM group which is enriched for genes involved in immune processes. Many targeted therapeutic agents are undergoing clinical trials, most often in unselected TNBC patients.

However, the application of precision medicine to TNBC has been limited so far by the lack of paired biomarkers and associated targeted therapy. While TNBC sub-types have been described in the literature, prospective biomarker-driven clinical trials are still uncommon in this disease. The identification of patient subgroups that show sensitivity to a specific treatment could result in more efficacious treatment for the biomarker-positive patients while avoiding unnecessary treatment (and their potential side effects) for biomarker-negative patients. Such targeted therapy could improve the therapeutic choices for TNBC patients. The present disclosure identifies biomarkers associated with response to TGFβ blockers and/or CPI. Biomarker positive patients are predicted to be more likely to respond to the therapy than biomarker negative patients.

US patent application publication number US 20150225483 A1, incorporated herein by reference, describes a bi-functional fusion protein that combines an anti-programmed death ligand 1 (PD-L1) antibody with the soluble extracellular domain of tumor growth factor beta receptor type II (TGFβRII) as a TGFβ neutralizing “Trap,” into a single molecule. Specifically, the protein is a heterotetramer, consisting of the two immunoglobulin light chains of anti-PD-L1, and two heavy chains comprising the heavy chain of anti-PD-L1 genetically fused via a flexible glycine-serine linker to the extracellular domain of the human TGFβRII (see FIG. 1). This anti-PD-L1/TGFβ Trap molecule is designed to target two major mechanisms of immunosuppression in the tumor microenvironment. US patent application publication number US 20150225483 A1 describes administration of the Trap molecule at doses based on the patient's weight.

The present disclosure provides methods for treating a subject diagnosed with TNBC, wherein the subject has been first determined to have an increased expression level of high mobility group AT-hook 2 (HMGA2) and/or MDS1 and EVI1 complex locus protein EVI1 (MECOM) relative to a known control expression level, and then administering anti-PD-L1/TGFβ Trap protein to the subject.

### SUMMARY OF THE DISCLOSURE

For an effective treatment of patients diagnosed with TNBC, the present disclosure provides a therapeutic regimen that treats TNBC in patients determined to have an increased expression level of HMGA2 or MECOM relative to a known expression level, and improves disease prognosis and overall survival of TNBC patients.

In one aspect, the present invention provides a method of treating or managing TNBC in a subject by administering an anti-TGFβ agent to a subject who has been determined to have an increased level of high mobility group AT-hook 2 (HMGA2) or MDS1 and EVI1 complex locus (MECOM) expression relative to a corresponding known control level, and thereby treating TNBC in the subject.

In another aspect, the present invention provides a method of achieving at least a partial response in treating or increasing survival of a triple negative breast cancer (TNBC) patient by administering an anti-TGFβ agent to a subject who has been determined to have an increased level of high mobility group AT-hook 2 (HMGA2) expression or MDS1 and EVI1 complex locus (MECOM) relative to a corresponding known control level, and thereby achieving at least a partial response in treating or increasing survival of a triple negative breast cancer (TNBC) patient.

In another aspect, the present invention provides a method of identifying a subject suitable for treating or managing TNBC in the subject with an anti-TGFβ agent, the method comprising determining the level of high mobility group AT-hook 2 (HMGA2) or MDS1 and EVI1 complex locus (MECOM) in the subject, wherein an increased level of HMGA2 or MECOM expression in the subject, relative to a corresponding known control level, identifies the subject as suitable for treating TNBC with the anti-TGFβ agent.

In certain embodiments, the present disclosure provides a two-step method of treating or managing TNBC in a subject, in which the first step involves identifying a subject who has an increased level of HMGA2 or MECOM expression relative to a corresponding known control level, and the second step involves administering an anti-TGFβ agent to the subject who has been determined to have an increased level of HMGA2 or MECOM, and thereby treating TNBC in the subject.

In certain embodiments, the present disclosure provides a two-step method of achieving at least a partial response in treating or increasing survival of a triple negative breast cancer (TNBC) patient, in which the first step involves identifying a subject who has an increased level of HMGA2 or MECOM expression relative to a corresponding known control level, and the second step involves administering an anti-TGFβ agent to the subject who has been determined to have an increased level of HMGA2 or MECOM, and thereby achieving at least a partial response in treating or increasing survival of a triple negative breast cancer (TNBC) patient.

In certain embodiments, the present disclosure provides a method of identifying a subject responsive to treatment of TNBC in the subject with an anti-TGFβ agent, in which the level of HMGA2 or MECOM in the subject is determined, and wherein an increased level of HMGA2 or MECOM expression in the subject, relative to a corresponding known control level, identifies the subject as suitable for treating TNBC with the anti-TGFβ agent.

In each of the methods of the present invention, the HMGA2 or MECOM level of the subject is determined by analyzing a tissue sample from the patient. In certain embodiments, the tissue sample is a biopsy sample, blood, serum, or plasma sample.

In each of the methods of the present invention, the level of HMGA2 or MECOM is determined by immunochemistry or by RNA expression analysis.

In some embodiments, the anti-TGFβ agent is an anti-PD-L1/TGFβ Trap protein used in the treatment of TNBC patients, comprising a first polypeptide comprising: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), and a second polypeptide comprising at least a variable region of a light chain of an antibody that binds PD-L1; wherein the heavy chain of the first polypeptide and the light chain of the second polypeptide, when combined, form an antigen binding site that binds PD-L1. In some embodiments, an anti-PD-L1/TGFβ Trap protein comprises a first polypeptide comprising the amino acid sequences of SEQ ID NOs: 35, 36, and 37, and a second polypeptide comprising the amino acid sequences of SEQ ID NOs: 38, 39, and 40. In some embodiments, an anti-PD-L1/TGFβ Trap protein comprises a first polypeptide comprising the amino acid sequence of SEQ ID NO: 3, and the second polypeptide comprising the amino acid sequence of SEQ ID NO: 1.

In certain embodiments, the subject who has been determined to have an increased level of HMGA2 or MECOM expression relative to a corresponding known control level is administered at least 1200 mg of the anti-PD-L1/TGFβ Trap protein. In certain embodiments, the subject who has been determined to have an increased level of HMGA2 or MECOM expression relative to a corresponding known control level is administered at least 1800 mg of the anti-PD-L1/TGFβ Trap protein. In certain embodiments, the subject who has been determined to have an increased level of HMGA2 or MECOM expression relative to a corresponding known control level is administered 1800 mg to 3000 mg of the anti-PD-L1/TGFβ Trap protein. In certain embodiments, the subject who has been determined to have an increased level of HMGA2 or MECOM expression relative to a corresponding known control level is administered 1200 mg to 2400 mg of the anti-PD-L1/TGFβ Trap protein.

In certain embodiments, the subject who has been determined to have an increased level of HMGA2 or MECOM expression relative to a corresponding known control level is administered 1200 mg of the anti-PD-L1/TGFβ Trap protein. In certain embodiments, the subject who has been determined to have an increased level of HMGA2 or MECOM expression relative to a corresponding known control level is administered 1200 mg of the anti-PD-L1/TGFβ Trap protein, once every two weeks.

In certain embodiments, the subject who has been determined to have an increased level of HMGA2 or MECOM expression relative to a corresponding known control level is administered 1800 mg of the anti-PD-L1/TGFβ Trap protein, once every three weeks.

In certain embodiments, the subject who has been determined to have an increased level of HMGA2 or MECOM expression relative to a corresponding known control level is administered 2400 mg of the anti-PD-L1/TGFβ Trap protein. In certain embodiments, the subject who has been determined to have an increased level of HMGA2 or MECOM expression relative to a corresponding known control level is administered 2400 mg of the anti-PD-L1/TGFβ Trap protein, once every three weeks. In certain embodiments, the subject who has been determined to have an increased level of HMGA2 or MECOM expression relative to a corresponding known control level is administered 2100 mg or 3000 mg of the anti-PD-L1/TGFβ Trap protein, once every three weeks.

In certain embodiments, in the methods of the present invention, the increased HMGA2 expression is at least 2.0-fold, for example, 2.27 more than a known population mean among TNBC patients. In certain embodiments, in the methods of the present invention, the increased HMGA2 expression is at least 2-5-fold more than the known population average level of HMGA2 expression. In certain embodiments, in the methods of the present invention, the increased HMGA2 expression is at least 200% higher, at least 300% higher, at least 400% higher, at least 500% higher, at least 600% higher, at least 700% higher, at least 800% higher, at least 900% higher, at least 1000% higher, or more than the normal level of HMGA2 expression. In certain embodiments, in the methods of the present invention, the increased HMGA2 expression in the subject is at least 19- to 35-fold more than the HMGA2 expression in a subject who is non-responsive to a treatment with an anti-TGFβ agent. In some embodiments, the expression level of the HMGA2 gene is measured by quantifying HMGA2 mRNA transcript normalized to house-keeping gene or genes. HMGA2 mRNA transcript and the house-keeping gene or genes are quantified by RNA quantitative methods, such as quantitative reverse transcription PCR. The house-keeping gene or genes are those that have relatively constant expression among the target population.

In certain embodiments, in the methods of the present invention, the increased MECOM expression is at least 1.5-fold more than a known population mean among TNBC patients. In certain embodiments, in the methods of the present invention, the increased MECOM expression is at least 1.5 to 4-fold more than the known population mean among TNBC patients.

In certain embodiments, in the methods of the present invention, the increased MECOM expression is at least 100%, at least 200% higher, at least 300% higher, at least 400% higher, at least 500% higher, at least 600% higher, at least 700% higher, at least 800% higher, at least 900% higher, at least 1000% higher, or more than the normal level of MECOM expression.

Determination of mRNA can be from tumor tissue or circulating tumor cells, or circulating tumor mRNA. The mRNA will be used to detect high expression of the HMGA2 or MECOM genes. High expression can be considered as tumor cells expressing a level above a certain reference level by PCR or other technologies that quantify mRNA expression.

In certain embodiments, in the methods of the present invention, the increased HMGA2 or MECOM expressions are determined via quantification of the HMGA2 and MECOM mRNA, respectively. In certain embodiments, in the methods of the present invention, HMGA2 mRNA or MECOM mRNA levels are determined via reverse transcription polymerase chain reaction (RT-qPCR) assay. In certain embodiments, HMGA2 mRNA or MECOM mRNA levels are determined via digital droplet PCR (ddPCR). In certain embodiments, in the methods of the present invention, the increased HMGA2 or MECOM expressions are determined via quantification of the HMGA2 and MECOM protein, respectively. In certain embodiments, in the methods of the present invention, the increased HMGA2 protein or MECOM protein levels are determined via immunohistochemistry. In certain embodiments, in the methods of the present invention, more than 1% tumor cells (e.g., more than 5%, more than 10%, more than 15%, or more than 20%) expressing HMGA2 protein in a tissue sample obtained from the TNBC subject determined the increased HMGA2 protein expression level. In certain embodiments, in the methods of the present invention, more than 1% tumor cells (e.g., more than 5%, more than 10%, more than 15%, or more than 20%) expressing MECOM protein in a tissue sample obtained from the TNBC subject determined the increased MECOM protein expression level.

Other embodiments and details of the disclosure are presented herein below.

### BRIEF DESCRIPTION OF THE DRAWINGS

FIG. 1 is a schematic drawing of an anti-PD-L1/TGFβ Trap molecule including one anti-PD-L1 antibody fused to two extracellular domains (ECDs) of TGFβ Receptor TI via a (Gly₄Ser)₄Gly (SEQ ID NO: 11) linker.

FIG. 2A is a box plot showing results for HMGA2 from the study described in Example 1. HMGA2 expression levels are plotted against response to anti-PD-L1/TGFβ Trap protein. TPM=transcripts per million; PD=progressive disease; SD=stable disease; PR=partial response. High HMGA2 expression is considered as expression level at least as high as the lowest HMGA2 expression among patients who responds to anti-PD-L1/TGFβ Trap protein treatment.

FIG. 2B is a histogram of the distribution of HMGA2 expression in breast cancer in the TCGA database. TPM=transcripts per million; NE=not evaluable. NE data was excluded from hypothesis testing.

FIG. 3A is a histogram of the distribution of MECOM expression in breast cancer in the TCGA database. TPM=transcripts per million; NE=not evaluable. High MECOM expression is considered as expression level at least as high as the lowest MECOM expression among patients who responds to anti-PD-L1/TGFβ Trap protein treatment.

FIG. 3B is a box plot showing results for MECOM from the study described in Example 1. MECOM expression levels are plotted against response to anti-PD-L1/TGFβ Trap protein. TPM=transcripts per million; PD=progressive disease; SD=stable disease; PR=partial response. High MECOM expression is considered as expression level at least as high as the lowest MECOM expression among patients who responds to anti-PD-L1/TGFβ Trap protein treatment.

FIGS. 4A-D show box plots of log-TPM of several potential predictive biomarkers plotted against response status of patients treated with anti-PD-L1/TGFβ Trap protein. FIG. 4A shows a box plot of log-TPM of HMGA2 plotted against response status of patients treated with anti-PD-L1/TGFβ Trap protein. FIG. 4B shows a box plot of log-TPM of MECOM plotted against response status of patients treated with anti-PD-L1/TGFβ Trap protein. FIG. 4C shows a box plot of log-TPM of CLEC3 A plotted against response status of patients treated with anti-PD-L1/TGFβ Trap protein. FIG. 4D shows a box plot of log-TPM of CCNDBP1 plotted against response status of patients treated with anti-PD-L1/TGFβ Trap protein. (Abbreviations used in the figures: NE (not evaluable), PD=progressive disease; SD=stable disease; PR=partial response).

FIGS. 5A-F are scatter plots showing association between HMGA2 and selected TGF-β signaling core genes. FIG. 5A is a scatter plot showing association between HMGA2 expression and Tgfbr1 expression. FIG. 5B is a scatter plot showing association between HMGA2 and Tgfbr2 expression. FIG. 5C is a scatter plot showing association between HMGA2 expression and Smad3 expression. FIG. 5D is a scatter plot showing association between HMGA2 expression and Tgfb1 expression. FIG. 5E is a scatter plot showing association between HMGA2 expression and Tgfb2 expression. FIG. 5F is a scatter plot showing association between HMGA2 expression and Tgfb3 expression.

FIGS. 6A-F are scatter plots showing association between HMGA2 and selected TGF-β signaling target genes. FIG. 6A is a scatter plot showing association between HMGA2 expression and Col1a1 expression. FIG. 6B is a scatter plot showing association between HMGA2 expression and Col1a2 expression. FIG. 6C is a scatter plot showing association between HMGA2 expression and Fn1 expression. FIG. 6D is a scatter plot showing association between HMGA2 expression and Vim expression. FIG. 6E is a scatter plot showing association between HMGA2 expression and Vegfa expression. FIG. 6F is a scatter plot showing association between HMGA2 expression and Zeb1 expression.

FIG. 7A is a plot showing expression of Tgfbr1 in control, Trap control, anti-PD-L1, and anti-PD-L1/TGFβ Trap-treated animal groups. FIG. 7B is a plot showing expression of Tgfbr2 in control, Trap control, anti-PD-L1, and anti-PD-L1/TGFβ Trap-treated animal groups. FIG. 7C is a plot showing expression of Smad3 in control, Trap control, anti-PD-L1, and anti-PD-L1/TGFβ Trap-treated animal groups. FIG. 7D is a plot showing expression of Tgfb1 in control, Trap control, anti-PD-L1, and anti-PD-L1/TGFβ Trap-treated animal groups. FIG. 7E is a plot showing expression of Tgfb2 in control, Trap control, anti-PD-L1, and anti-PD-L1/TGFβ Trap-treated animal groups. FIG. 7F is a plot showing expression of Tgfb3 in control, Trap control, anti-PD-L1, and anti-PD-L1/TGFβ Trap-treated animal groups.

FIG. 8A is a plot showing expression of HMGA2 in control, Trap control, anti-PD-L1, and anti-PD-L1/TGFβ Trap-treated animal groups. FIG. 8B is a plot showing expression of Col1a1 in control, Trap control, anti-PD-L1, and anti-PD-L1/TGFβ Trap-treated animal groups. FIG. 8C is a plot showing expression of Col1a2 in control, Trap control, anti-PD-L1, and anti-PD-L1/TGFβ Trap-treated animal groups. FIG. 8D is a plot showing expression of Fn1 in control, Trap control, anti-PD-L1, and anti-PD-L1/TGFβ Trap-treated animal groups. FIG. 8E is a plot showing expression of Vim in control, Trap control, anti-PD-L1, and anti-PD-L1/TGFβ Trap-treated animal groups. FIG. 8F is a plot showing expression of Vegfa in control, Trap control, anti-PD-L1, and anti-PD-L1/TGFβ Trap-treated animal groups. FIG. 8G is a plot showing expression of Zeb1 in control, Trap control, anti-PD-L1, and anti-PD-L1/TGFβ Trap-treated animal groups.

FIG. 9A is a scatter plot showing association between HMGA2 expression and Ifhg expression. FIG. 9B is a scatter plot showing association between HMGA2 expression and Gzmb expression. FIG. 9C is a scatter plot showing association between HMGA2 expression and Gzmk expression. FIG. 9D is a plot showing expression of Ifhg in control, trap control, Anti-PD-L1, and anti-PD-L1/TGFβ Trap-treated animals. FIG. 9E is a plot showing expression of Gzmb in control, trap control, Anti-PD-L1, and anti-PD-L1/TGFβ Trap-treated animals. FIG. 9F is a plot showing expression of Gzmk in control, trap control, Anti-PD-L1, and anti-PD-L1/TGFβ Trap-treated animals.

FIG. 10 is a box plot showing expression of HMGA2 (in log-TPM (transcript per million) and TNBC status of subjects.

FIG. 11 shows HMGA2 expression (in TPM) versus response in separate panels for non-TNBC (left) and for TNBC (right) subjects. [Abbreviations used in the figure: NE: not evaluable; PD: progressive disease; SD: stable disease; PR: partial response; CR: complete response; METBRC: metastatic breast cancer; TPM: transcript per million for the HMGA2 gene].

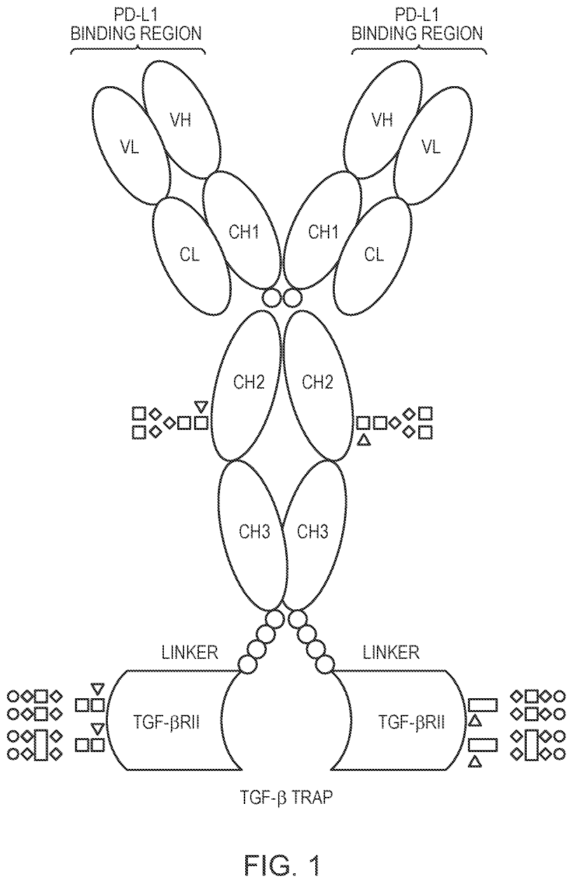

### DETAILED DESCRIPTION

By “TGFβRII” or “TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ TD NO: 8)), or a polypeptide having the wild-type human TGFβ Receptor Type 2 Isoform B sequence (e.g., the amino acid sequence of NCBI RefSeq Accession No. NP 003233 (SEQ ID NO: 9)) or having a sequence substantially identical to the amino acid sequence of SEQ ID NO: 8 or of SEQ ID NO: 9. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII lacks the signal sequence.

By a “fragment of TGFβRII capable of binding TGFβ” is meant any portion of NCBI RefSeq Accession No. NP_001020018 (SEQ ID NO: 8) or of NCBI RefSeq Accession No. NP_003233 (SEQ ID NO: 9), or a sequence substantially identical to SEQ ID NO: 8 or SEQ ID NO: 9 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 10.

“PD-L1 high” or “high PD-L1” refers to ≥80% PD-L1 positive tumor cells as determined by the PD-L1 IHC 73-10 assay (Dako), or tumor proportion score (TPS)≥50% as determined by the Dako IHC 22C3 PharmDx assay (TPS is a term of art related to the IHC 22C3 PharmDx assay, which describes the percentage of viable tumor cells with partial or complete membrane staining (e.g., staining for PD-L1)). Both IHC 73-10 and IHC 22C3 assays select a similar patient population at their respective cutoffs. In certain embodiments, VENTANA PD-L1 (SP263) assay, which has high concordance with 22C3 PharmDx assay (see Sughayer et al., Appl. Immunohistochem. Mol. Morphol., (2018)), can also be used for determining PD-L1 high expression level.

“PD-L1 positive” or “PD-L1+” indicates TPS≥1% PD-L1 positive tumor cells as determined, for example, by the Dako PD-L1 IHC 22C3 pharmDx assay.

By “substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

By “patient” is meant either a human or non-human animal (e.g., a mammal). “Patient,” “subject,” “patient in need thereof,” and “subject in need thereof” are used interchangeably in the present disclosure, and refer to a living organism suffering from or prone to a disease or condition that can be treated by administration using the methods and compositions provided in the present disclosure.

The terms “treat,” “treating,” or “treatment,” and other grammatical equivalents as used in the present disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The term “consolidation” in the context of a therapeutic regimen of the present disclosure is used as is commonly understood in the art. For example, according to the National Cancer Institute, the term “consolidation therapy” is a “[t]reatment that is given after cancer has disappeared following the initial therapy. Consolidation therapy is used to kill any cancer cells that may be left in the body. It may include radiation therapy, a stem cell transplant, or treatment with drugs that kill cancer cells. Also called intensification therapy and postremission therapy.” https://www.cancer.gov/publications/dictionaries/cancer-terms/def/consolidation-therapy, last visited on Jun. 9, 2018.

The term “progression-free survival” or PFS is defined as the time from randomization (which can occur 6 or more weeks after treatment initiation) to the date of the first documented event of tumor progression or death in the absence of disease progression. The term “overall survival” is defined as the time from randomization until death from any cause. Progression-free survival is assessed by the investigators, according to RECIST, version 1.1, as a predefined sensitivity analysis.

By “cancer” is meant triple negative breast cancer (TNBC), for which immunohistochemistry has confirmed that the breast cancer does not express estrogen receptor (ER) and progesterone receptor (PR) at all, and also does not overexpress HER2.

By “advanced triple negative breast cancer (TNBC)” is meant metastatic disease, treatment-refractory disease, or cancer that was previously locally advanced and now has progressed.

By “responsive” subject or “responder,” it is meant that a subject with TNBC receiving treatment with anti-PD-L1/TGFβ Trap protein will experience a best overall response of at least a partial response (PR), or a complete response (CR) as determined by RECIST 1.1.

By “non-responsive” subject or “non-responder,” it is meant that a subject with TNBC receiving treatment with anti-PD-L1/TGFβ Trap protein will experience a best overall response of progressive disease (PD) as determined by RECIST 1.1.

The terms “risk,” “at risk,” and “risk factor,” are used here as conventionally understood in the art. For example, a risk factor is any attribute, characteristic or exposure of an individual that increases the likelihood of developing a disease or injury. In certain embodiments, a person at risk of developing a disease, disorder, or condition means that the person is exposed to a risk factor that contributes or enhances the probability of incidence of that disease, disorder, or condition.

Throughout the description and claims of the present disclosure the word “comprise” and other forms of the word, such as “comprising” and “comprises,” means including but not limited to, and is not intended to exclude, for example, other components.

By “co-administer” it is meant that a composition described herein is administered at the same time, just prior to, or just after the administration of additional therapies. The protein and the composition of the present disclosure can be administered alone or can be co-administered with a second, third, or fourth therapeutic agent(s) to a patient. Co-administration is meant to include simultaneous or sequential administration of the protein or composition individually or in combination (more than one therapeutic agent).

The term “a” is not meant to limit as a singular. In certain embodiments, the term “a” may refer to a plural form. As used throughout the present disclosure, the singular forms “a,” “an,” and “the” include plural reference unless the context clearly dictates otherwise. Thus, for example, a reference to “a composition” includes a plurality of such compositions, as well as a single composition.

A “reconstituted” formulation is one which has been prepared by dissolving a lyophilized formulation in an aqueous carrier such that the bifunctional molecule is dissolved in the reconstituted formulation. The reconstituted formulation is suitable for intravenous administration (IV) to a patient in need thereof.

The term “about” refers to any minimal alteration in the concentration or amount of an agent that does not change the efficacy of the agent in preparation of a formulation and in treatment of a disease or disorder. In embodiments, the term “about” may include ±15% of a specified numerical value or data point.

Ranges can be expressed in the present disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in the present disclosure, and that each value is also disclosed as “about” that particular value in addition to the value itself. It is also understood that throughout the application, data are provided in a number of different formats and that the data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

An “isotonic” formulation is one which has essentially the same osmotic pressure as human blood. Isotonic formulations will generally have an osmotic pressure from about 250 to 350 mOsmol/kgH₂O. The term “hypertonic” is used to describe a formulation with an osmotic pressure above that of human blood. Isotonicity can be measured using a vapor pressure or ice-freezing type osmometer, for example.

The term “buffering agent” refers to one or more components that when added to an aqueous solution is able to protect the solution against variations in pH when adding acid or alkali, or upon dilution with a solvent. In addition to phosphate buffers, there can be used glycinate, carbonate, citrate buffers and the like, in which case, sodium, potassium or ammonium ions can serve as counterion.

An “acid” is a substance that yields hydrogen ions in aqueous solution. A “pharmaceutically acceptable acid” includes inorganic and organic acids which are nontoxic at the concentration and manner in which they are formulated.

A “base” is a substance that yields hydroxyl ions in aqueous solution. “Pharmaceutically acceptable bases” include inorganic and organic bases which are non-toxic at the concentration and manner in which they are formulated.

A “lyoprotectant” is a molecule which, when combined with a protein of interest, prevents or reduces chemical and/or physical instability of the protein upon lyophilization and subsequent storage.

A “preservative” is an agent that reduces bacterial action and may be optionally added to the formulations herein. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3pentanol, and m-cresol.

A “surfactant” is a surface active molecule containing both a hydrophobic portion (e.g., alkyl chain) and a hydrophilic portion (e.g., carboxyl and carboxylate groups). Surfactant may be added to the formulations of the invention. Surfactants suitable for use in the formulations of the present invention include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.).

For an effective treatment of patients diagnosed with triple negative breast cancer (TNBC), the present disclosure provides a therapeutic regimen that treats TNBC in patients determined to have an increased expression level of HMGA2 or MECOM relative to a known expression level, and improves disease prognosis and overall survival of TNBC patients. The known expression level is the HMGA2 expression level in a control population or tissue sample. In certain embodiments, patient is diagnosed with advanced TNBC. In certain embodiments, patient is diagnosed with metastatic TNBC refractory to prior lines of treatment.

Anti-TGFβ agents of the present disclosure include TGFβ traps, antibodies, small molecule inhibitors, and oligonucleotides targeting TGFβ expression. For example, anti-TGFβ agents include TGFβ-neutralizing antibodies ID11, 2G7, Fresolimumab (GC1008; Sanofi, Genzyme), Metelimumab (CAT-192; AstraZeneca, Cambridge Antibody Technology), TGFβ receptor-blocking antibodies such as LY3022859 (Eli Lilly & Co) and small molecule TGFβ receptor kinase inhibitor Galunisertib (LY2157299; Eh Lilly & Co), SD-208 (Scios Inc), and LY2109761 (Eh Lilly & Co.).

The bifunctional protein of the present disclosure (anti-PD-L1/TGFβ Trap molecule) includes a first and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ) (e.g., a soluble fragment). The second polypeptide includes at least a variable region of a light chain of an antibody that binds PD-L1, in which the heavy chain of the first polypeptide and the light chain of the second polypeptide, when combined, form an antigen binding site that binds PD-L1 (e.g., any of the antibodies or antibody fragments described herein). Because the bifunctional protein of the present disclosure binds to two targets, (1) PD-L1, which is largely membrane bound, and (2) TGFβ, which is soluble in blood and interstitium, the BW-independent dosing regimen requires a dose that is effective not only to inhibit PD-L1 at the tumor site but also sufficient to inhibit TGFβ.

### Method of Treating Cancer or Inhibiting Tumor Growth

In one aspect, the present invention provides a method of treating or managing TNBC in a subject by administering an anti-TGFβ agent to a subject who has been determined to have an increased level of high mobility group AT-hook 2 (HMGA2) or MDS1 and EVI1 complex locus (MECOM) expression relative to a corresponding known control level, and thereby treating TNBC in the subject.

In another aspect, the present invention provides a method of achieving at least a partial response in treating or increasing survival of a TNBC patient by administering an anti-TGFβ agent to a subject who has been determined to have an increased level of high mobility group AT-hook 2 (HMGA2) expression or MDS1 and EVI1 complex locus (MECOM) relative to a corresponding known control level, and thereby achieving at least a partial response in treating or increasing survival of a triple negative breast cancer (TNBC) patient.

In certain embodiments, the present disclosure provides a two-step method of treating or managing TNBC in a subject, in which the first step involves identifying a subject who has an increased level of HMGA2 or MECOM expression relative to a corresponding known control level, and the second step involves administering an anti-TGFβ agent to the subject who has been determined to have an increased level of HMGA2 or MECOM, and thereby treating TNBC in the subject.

In certain embodiments, the present disclosure provides a two-step method of achieving at least a partial response in treating or increasing survival of a triple negative breast cancer (TNBC) patient, in which the first step involves identifying a subject who has an increased level of HMGA2 or MECOM expression relative to a corresponding known control level, and the second step involves administering an anti-PD-L1/TGFβ Trap protein to the subject who has been determined to have an increased level of HMGA2 or MECOM, and thereby achieving at least a partial response in treating or increasing survival of a triple negative breast cancer (TNBC) patient.

In each of the methods of the present invention, the HMGA2 or MECOM level of the subject is determined by analyzing a tissue sample from the patient. In certain embodiments, the tissue sample is a blood, serum, or plasma sample. In particular embodiments, the tissue sample from the subject is a breast tissue obtained by a biopsy (e.g., needle biopsy sample collected from the patient before initiation of treatment). In each of the methods of the present invention, the level of HMGA2 or MECOM is determined by immunochemistry of biopsy sample or by RNA expression analysis of a biopsy sample or blood, serum, or plasma sample collected from the patient before initiation of treatment.

In particular embodiments, the tissue sample from the subject is a breast cancer tissue obtained by a biopsy (e.g., needle biopsy sample collected from the patient before initiation of treatment). In each of the methods of the present invention, the level of HMGA2 or MECOM is determined by immunochemistry of biopsy sample or by RNA expression analysis of a biopsy sample or blood, serum, or plasma sample collected from the patient before initiation of treatment. In certain embodiments, in the methods of the present invention, the increased HMGA2 mRNA and MECOM mRNA levels are determined via well-known mRNA quantification methods. In an exemplary embodiment, in the methods of the present invention, HMGA2 mRNA and MECOM mRNA levels are determined via reverse transcription polymerase chain reaction (RT-qPCR) assay. In an exemplary embodiment, HMGA2 mRNA and MECOM mRNA levels are determined via digital droplet PCR (ddPCR).

In certain embodiments, the present disclosure provides a method for predicting the response of a patient diagnosed with triple negative breast cancer to an anti-TGFβ agent and of treating the patient by administering an anti-TGFβ agent. In an exemplary embodiment, the level of HMGA2 is determined by extracting RNA from fresh paraffin embedded tumor sample. In an exemplary embodiment, the level of HMGA2 is determined by extracting RNA from frozen paraffin embedded tumor sample. In another exemplary embodiment, the level of HMGA2 is determined by extracting RNA from fixed paraffin embedded tumor sample. In certain embodiments, the extracted RNA is reverse transcribed to produce cDNA. In certain embodiments, the reverse transcribed cDNA is amplified using a PCR-based method (e.g., RT-qPCR, digital droplet PCR). In an exemplary embodiment, a PCR-based method (e.g., RT-qPCR, digital droplet PCR) is utilized to quantitatively assay RNA transcript levels of HMGA2 expression. In certain embodiments, HMGA2 RNA transcript level is normalized against a level of RNA transcript of at least one housekeeping gene to provide a normalized HMGA2 transcript level. Methods for normalizing gene expression data using well-known housekeeping genes (e.g., GAPDH, actin, ubiquitin) are well-known (see Dheda K. et. al., Validation of housekeeping genes for normalizing RNA expression in real-time PCR. Biotechniques 2004; 37: 112-119.).

In certain embodiments, the present invention involves administering an anti-PD-L1/TGFβ Trap protein to the subject who has been determined to have an increased level of HMGA2 RNA transcript levels compared to the distribution of HMGA2 RNA transcript levels across all tumors in the population, and thereby achieving at least a partial response in treating or increasing survival of a triple negative breast cancer (TNBC) patient.

In certain embodiments, a high HMGA2 expression cutoff is set based on the quantitation method used to quantify HMGA2 expression in the subject. In an exemplary embodiment, HMGA2 high expression cutoff to select patient population that will respond to anti-PD-L1/TGFβ Trap protein treatment is deduced by incorporation of data obtained from RNA-seq and data obtained from qPCR and/or ddPCR. The TPM values obtained from RNA-seq may be translated into quantitation values that can be obtained from absolute quantitation methods (e.g., qPCR or ddPCR). A transfer function that maps from TPM values obtained from RNA-seq to Ct values (for qPCR) or ddPCR ratio values (for ddPCR) is generated. This transfer function is used to find the corresponding Ct or ddPCR ratio levels that can provide a cutoff with regards to high HMGA2 expression. In an exemplary embodiment, transfer function used to find corresponding Ct values that can provide a cutoff with regards to high HMGA2 expression is:

Y₁=X₁−log 2(TPM_{lowest}/TPM_{baseline});

where Y₁=Ct value cutoff;

X₁=normalized ΔCt value (median relative qPCR expression for HMGA2);

TPM_{lowest}=lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In an exemplary embodiment, transfer function used to find corresponding Ct values that can provide a cutoff with regards to high HMGA2 expression is:

Y₁=X₁−log 2(TPM_{second lowest}/TPM_{baseline});

where Y₁=Ct value cutoff;

X₁=normalized ΔCt value (median relative qPCR expression for HMGA2);

TPM_{second lowest}=lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In certain embodiments, transfer function used to find corresponding ddPCR ratio values that can provide a cutoff with regards to high HMGA2 expression is:

Y₁=X₁×(TPM_{lowest}/TPM_{baseline});

where Y₁=ddPCR ratio value cutoff;

X₁=normalized ddPCR ratio value (median ddPCR ratio value for HMGA2);

TPM_{lowest}=lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In certain embodiments, transfer function used to find corresponding ddPCR ratio values that can provide a cutoff with regards to high HMGA2 expression is:

Y₁=X₁×(TPM_{second lowest}/TPM_{baseline});

where Y₁=ddPCR ratio value cutoff;

X₁=normalized ddPCR ratio value (median ddPCR ratio value for HMGA2);

TPM_{second lowest}=second lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In an exemplary embodiment, a patient is regarded as HMGA2 high when the expression of HMGA2 in that subject's tumor is high with respect to the distribution of HMGA2 expression across all tumors in the TNBC population. In an exemplary embodiment, the reference level of HMGA2 expression is determined by collecting tumor samples of a cohort of patients who responded to treatment with anti-PD-L1/TGFβ Trap and a cohort of patients who did not respond (responders and non-responders, respectively); measuring HMGA2 expression within the samples; characterizing the distributions of expression among responders and non-responders reflected in a cut-off value separating these distributions; and setting the threshold value that corresponded to a selected cut-off between the responders and non-responders as the reference expression level.

In one aspect, the present invention provides a method for identifying a TNBC patient likely to respond (e.g., partial response (PR), improved survival) to treatment with targeted TGFβ inhibition. To identify a TNBC patient likely to respond to treatment with anti-TGFβ agent, the expression levels of HMGA2 and/or MECOM relative to a corresponding known control expression level, respectively, is analyzed. In certain embodiments, the analysis of HMGA2 or MECOM expression levels is performed 7-30 days (e.g., 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days) or more, before treatment with targeted TGFβ inhibition with an anti-PD-L1/TGFβ Trap molecule. In order to determine levels of HMGA2 and/or MECOM, in a TNBC patient using the methods described herein, a sample may be obtained from the patient. Therefore, in some embodiments of the invention, the level of HMGA2 and/or MECOM in the TNBC patient is determined in a sample obtained from the TNBC patient. In certain embodiments, to identify a TNBC patient likely to respond to treatment with an anti-TGFβ agent, the expression levels of HMGA2 and/or MECOM is analyzed in a tissue sample collected from the subject. In particular embodiments, the tissue sample from the subject is a blood, serum, or plasma sample. In particular embodiments, the tissue sample from the subject is abreast tissue obtained by a biopsy (e.g., needle biopsy sample collected from the patient before initiation of treatment).

In certain embodiments, the present invention provides a method of treating TNBC patients with high HMGA2 expression relative to a known control expression level of the general TNBC population with agents targeting TGFβ. In certain embodiments, agents targeting TGFβ can be small molecules, monoclonal antibodies, fusion proteins of TGFβ receptors, and/or antisense RNA derivatives. In certain embodiments, these agents are known to target TGFβ pathway. For example: Galunisertib (LY-2157299; Eli Lilly & Co.), vactosertib (TEW-7197, NOV-1301; MedPacto, Inc., National OncoVenture), LY3200882 (Eh Lilly & Co.), NIS-793 (XOMA-089; Novartis, XOMA corporation), SAR-439459 (Sanofi), ABBV-151 (AGRX-115, AbbVie, Argenx), AVID-200 (Forbius), PF-06952229 (Pfizer), and YL-13027 (Shanghai YingLi Pharmaceutical Co., Ltd.).

In some embodiments, the level of HMGA2 and/or MECOM is determined by analyzing a sample from the patient. In certain embodiments, the tissue sample from the subject is a breast tissue obtained by a biopsy (e.g., needle biopsy sample collected from the patient before initiation of treatment). In each of the methods of the present invention, the level of HMGA2 or MECOM is determined by immunochemistry of biopsy sample or by RNA expression analysis of a biopsy sample or blood, serum, or plasma sample collected from the patient before initiation of treatment. For example, in methods of the invention, the level of HMGA2 and/or MECOM may be determined by immunochemistry, for example, by an enzyme-linked immunosorbent assay (ELISA), or by nucleotide analysis.

In certain embodiments, the methods of the present invention involve comparing the measured levels of mRNA expression of HMGA2 and/or MECOM in a sample obtained from a TNBC patient, to the levels of the mRNA expression of known values available at the TCGA database (The Cancer Genome Atlas—National Institutes of Health), which is a database of a large number of patients.

Comparing RNA-seq datasets is non-trivial due to variations in technical platform and sample preparation leading to what are commonly called batch effects; to correct for the batch effects separating the observed levels of HMGA2 and/or MECOM expression from the levels according to TCGA, the ComBat algorithm was implemented in the sva Bioconductor package (sva version 3.28.0, Leek J T, Johnson W E, Parker H S, Fertig E J, Jaffe A E, Storey J D, Zhang Y, Torres L C (2018). sva: Surrogate Variable Analysis.). This method rendered comparable the patient clinical data in the TCGA breast cancer dataset (hereafter TCGA-BRCA) that were triple-negative (hereafter TCGA BRCA TNBC) to the observed levels of HMGA2 and/or MECOM.

In certain embodiments, to identify a TNBC patient likely to respond to treatment with anti-PD-L1/TGFβ Trap molecule, the expression level of HMGA2 or MECOM is analyzed by sequencing RNA extracted from formalin-fixed, paraffin-embedded (FFPE) tissue samples. For example, RNA is extracted from FFPE samples by isolating total RNA from one to two 5-10 μm FFPE curls by RNeasy FFPE kit (Qiagen, Hilden, Germany), and RNA concentration is determined using the Qubit HS RNA assay (ThermoFisher Scientific, USA) on the Qubit 2.0 fluorometer. The extracted RNA is then sequenced using methods known in the art. For example, in certain embodiments, quantitative real-time PCR (qPCR) is used to analyze HMGA2 or MECOM expression level. In certain embodiments, qPCR is performed in duplicate using TaqMan Gene Expression Master Mix and ran on Applied Biosystems 7500 Fast Real-Time PCR System (96-well format) using the manufacturer's recommended cycling protocol. Primer/probe set for target genes (SEQ ID NO: 63 and/or SEQ ID NO: 64) and house-keeping genes can be designed using Primer Express® if “off-the-shelf” gene expression assay is not available. The comparative ΔCt method may be used for relative quantification of gene expression.

In another embodiment, digital droplet PCR (ddPCR) is used to analyze HMGA2 or MECOM expression level. In certain embodiments, ddPCR is performed using assays containing primers and probes targeting target genes (SEQ ID NO: 63 and/or SEQ ID NO: 64) and house-keeping genes following BioRad ddPCR protocol. Sample analysis of each experiment is performed using QuantaSoft software. Positive droplet concentrations in all samples are determined using manually placed fluorescence thresholds based on negative clusters as detected in the corresponding no template control (NTCs). Target DNA concentration (copies/μL) and absolute droplet counts within single samples are used as the quantitative outcome measurement.

In another embodiment, the HTG EdgeSeq system is used to analyze HMGA2 or MECOM expression level. The FFPE specimens are scraped into tubes and lysed in HTG's lysis buffer, followed by the introduction of gene-specific DNA nuclease protection probes (NPP). After allowing the NPPs to hybridize to their target RNAs, which can be both soluble or cross-linked in the biological matrix, SI nuclease is added which removes excess unhybridized NPPs and RNAs, leaving behind only NPPs hybridized to their target RNAs.

Thus, a stoichiometric conversion of the target RNA to the NPPs is achieved, producing a virtual 1:1 ratio of NPP to RNA. The qNPA steps are automated on the HTG EdgeSeq processor, which is followed by PCR to add sequencing adaptors and tags. The labeled samples are pooled, cleaned, and sequenced on a next generation sequencing (NGS) platform using standard protocols. Data from the NGS instrument are processed and reported by the HTG EdgeSeq parser software.

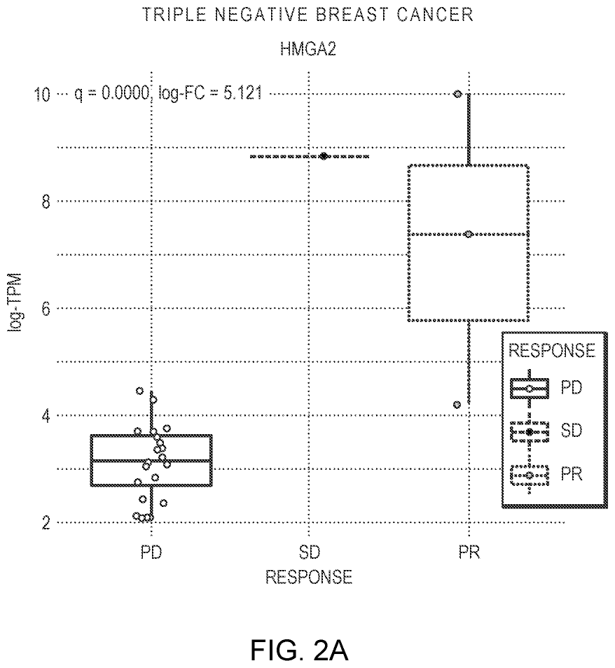

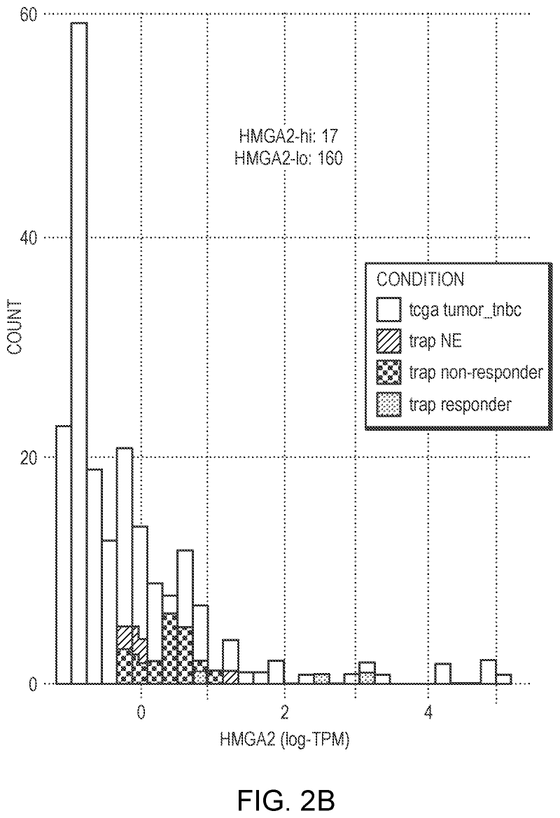

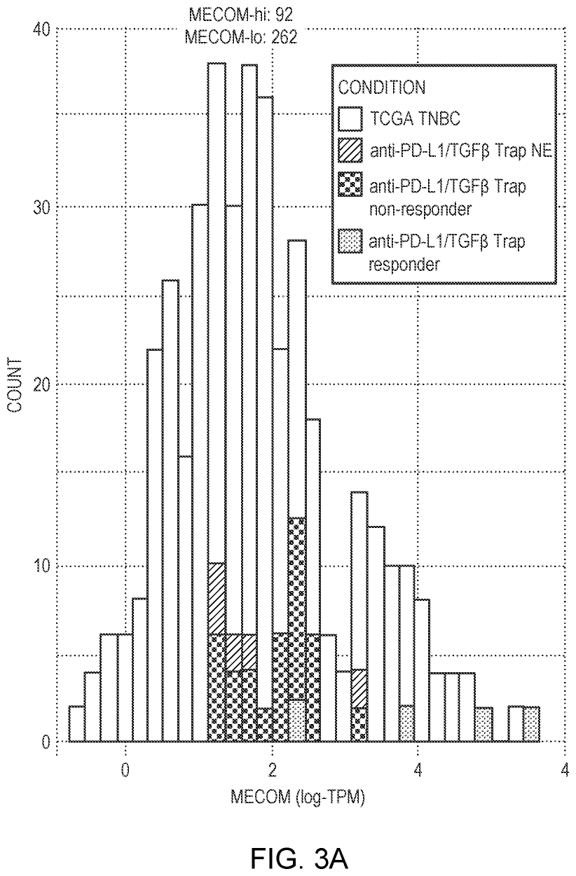

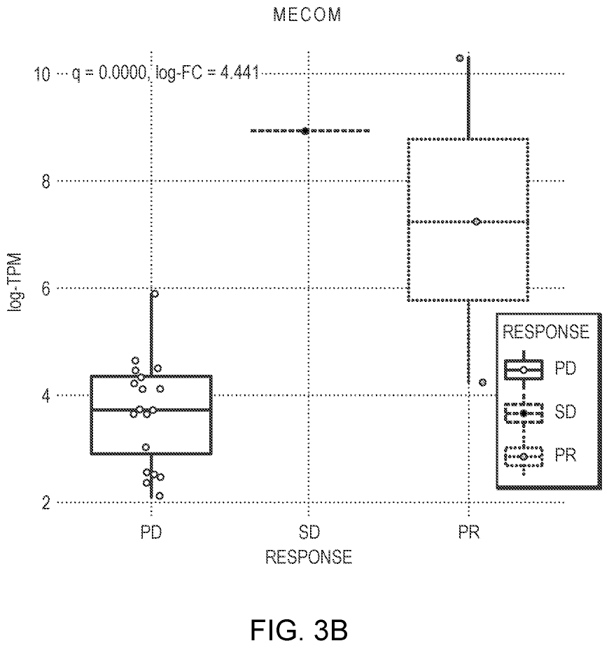

As shown in FIG. 2A (HMGA2) and FIG. 3B (MECOM), both HMGA2 and MECOM over-express by more than 20-fold in responders as compared with non-responders. In FIG. 2A, HMGA2 expression levels (log of transcripts per million (TPM)) are plotted against response to anti-PD-L1/TGFβ Trap protein (PD=progressive disease; SD=stable disease; PR=partial response). TPM, a widely used metric of transcript abundance, was computed by RSEM. See Li & Dewey, (2011), BMC Bioinformatics, 12:323. The high and low expression levels of the HMGA2 are noted in FIG. 2B, of MECOM are noted in FIG. 3A. The ratio of the high-to-low expression levels provides an independent value (a factor) by which TNBC patients who would likely respond to the anti-PD-L1/TGFβ Trap protein treatment could be identified.

High HMGA2 expression is an expression level, which is at least as high as the lowest HMGA2 expression among patients who responds to anti-PD-L1/TGFβ Trap protein treatment. As shown in FIG. 2A, HMGA2 expression is significantly higher (at least 35-fold) compared to the expression levels among the non-responders.

In FIG. 3B, MECOM expression levels (log of transcripts per million (TPM)) are plotted against response to treatment with an anti-PD-L1/TGFβ Trap protein (PD=progressive disease; SD=stable disease; PR=partial response). High MECOM expression is an expression level, which is at least as high as the lowest MECOM expression among patients who responds to anti-PD-L1/TGFβ Trap protein treatment. As shown in FIG. 3B, MECOM expression is significantly higher (at least 20-fold) compared to the expression levels among the non-responders.

In certain embodiments, the expression level of HMGA2 or MECOM is analyzed by immunohistochemistry (IHC). For example, in certain embodiments, an automated IHC method can be developed for assaying the expression of HMGA2 or MECOM in tumor cell nuclei in FFPE tissue specimens. The current disclosure provides methods for detecting the presence of human HMGA2 or MECOM antigen in a test tissue sample, or quantifying the level of human HMGA2 or MECOM antigen or the proportion of cells in the sample that express the antigen, which methods comprise contacting the test sample, and a negative control sample, with a mAb that specifically binds to human HMGA2 or MECOM, under conditions that allow for formation of a complex between the Ab or portion thereof and human HMGA2 or MECOM. Preferably, the test and control tissue samples are FFPE samples. The formation of a complex is then detected, wherein a difference in complex formation between the test sample and the negative control sample is indicative of the presence of human HMGA2 or MECOM antigen in the sample. Various methods are used to quantify HMGA2 or MECOM expression.

In a particular embodiment, the automated IHC method comprises: (a) deparaffinizing and rehydrating mounted tissue sections in an autostainer; (b) retrieving antigen in a Target Retrieval Solution, at the appropriate pH, using a pre-treatment module, (c) running the autostainer to include steps of neutralizing endogenous peroxidase in the tissue specimen; blocking non-specific protein-binding sites on the slides; incubating the slides with primary Ab or negative control reagent; incubating with a post-primary blocking agent; adding a chromogen substrate and developing; and counterstaining with hematoxylin, (d) dehydrating in graded ethanol series, and clearing prior to mounting with permanent medium.

For assessing HMGA2 or MECOM expression in tumor tissue samples, a pathologist examines the number of HMGA2+ or MECOM+ tumor cells in each field under a microscope and mentally estimates the percentage of cells that are positive, then averages them to come to the final percentage. The different staining intensities are designated as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%.

In certain embodiments of these scoring methods, the samples are scored by two pathologists operating independently and the scores are subsequently consolidated. In certain other embodiments, the identification of positive and negative cells is scored using appropriate software.

A histoscore is used as a more quantitative measure of the IHC data. The histoscore is calculated as follows: Histoscore=[(% tumor.times.1 (low intensity))+(% tumor.times.2 (medium intensity))+(% tumor.times.3 (high intensity)] To determine the histoscore, the percentage of stained cells in each intensity category within a specimen is estimated. The final histoscore range may be 0 (no expression) to 300 (maximum expression).

In certain embodiments, in the methods of the present invention, the increased HMGA2 expression is at least 2.0-fold, for example, 2.27 more than a known population mean among TNBC patients. In certain embodiments, in the methods of the present invention, the increased HMGA2 expression is at least 2 to 7-fold (e.g., 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 5.6-fold, 5.7-fold, 5.8-fold, 5.9-fold, 6.0-fold, 6.1-fold, 6.2-fold, 6.3-fold, 6.4-fold, 6.5-fold, 6.6-fold, 6.7-fold, 6.8-fold, 6.9-fold, or 7.0-fold) more than the known population average level of HMGA2 expression (e.g., population mean among TNBC patients).

In certain embodiments, in the methods of the present invention, the increased MECOM expression is at least 1.5-fold more than a known population mean among TNBC patients. In certain embodiments, in the methods of the present invention, the increased MECOM expression is at least 1.5 to 4-fold (e.g., 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, or 4.0-fold) more than the known population mean among TNBC patients.

In certain embodiments, the expression level of HMGA2 or MECOM is compared to the known expression level of the general TNBC population. In certain embodiments, HMGA2 expression level is determined to be high if the HMGA2 RNA expression level is determined to be greater than 2.60 times than tire HMGA2 RNA expression level of tire general TNBC population. In certain embodiments, in the methods of the present invention, more than 1% tumor cells (e.g., more than 5%, more than 10%, more than 15%, or more than 20%) expressing HMGA2 protein in a tissue sample obtained from the TNBC subject determined the increased HMGA2 protein expression level. In certain embodiments, MECOM expression level is determined to be high if the MECOM RNA expression level is greater than 1.7 times than the MECOM RNA expression level of the general TNBC population. In certain embodiments, in the methods of the present invention, more than tumor 1% cells (e.g., more than 5%, more than 10%, more than 15%, or more than 20%) expressing MECOM protein in a tissue sample obtained from the TNBC subject determined the increased MECOM protein expression level.

In certain embodiments, in the methods of the present invention, the increased HMGA2 expression in the subject is at least 19- to 40-fold (e.g., 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, or 40-fold) more than the HMGA2 expression in a subject who is non-responsive to a treatment with the anti-PD-L1/TGFβ Trap protein.

In certain embodiments, the expression level of HMGA2 or MECOM is compared between responders and non-responders within the TNBC population. In certain embodiments, HMGA2 expression level is determined to be high if the HMGA2 RNA expression level is determined to be at least 19 to 35 times greater than the HMGA2 RNA expression level of the non-responder TNBC population. In certain embodiments, MECOM expression level is determined to be high if the MECOM RNA expression level is greater than at least 18 to 35 times greater than the MECOM RNA expression level of the non-responder TNBC population.

In certain embodiments, in the methods of the present invention, the increased HMGA2 expression is at least 200% higher, at least 300% higher, at least 400% higher, at least 500% higher, at least 600% higher, at least 700% higher, at least 800% higher, at least 900% higher, at least 1000% higher, or more than the normal level of HMGA2 expression.

In certain embodiments, in the methods of the present invention, the increased HMGA2 and/or MECOM expression is 100%-1000% higher (200%-1000% higher, 300%-1000% higher, 400%-1000% higher, 500%-1000% higher, 600%-1000% higher, 700%-1000% higher, 800%-1000% higher, 900%-1000% higher, 100%-900% higher, 100%-800% higher, 100%-700% higher, 100%-600% higher, 100%-500% higher, 100%-400% higher, 100%-300% higher, or 100%-200% higher) transcript expression than the normal population level of HMGA2 and/or MECOM expression. In certain embodiments, the subject identified to be responsive to treatment with targeted TGFβ inhibition has been determined to have increased HMGA2 transcript expression, wherein the increased HMGA2 transcript expression is at least 200% higher, at least 300% higher, at least 400% higher, at least 500% higher, at least 600% higher, at least 700% higher, at least 800% higher, at least 900% higher, at least 1000% higher, or more, than the normal level of HMGA2 transcript expression. In certain embodiments, the subject identified to be responsive to treatment with targeted TGFβ inhibition has been determined to have increased MECOM expression, wherein the increased MECOM transcript expression is at least 200% higher, at least 300% higher, at least 400% higher, at least 500% higher, at least 600% higher, at least 700% higher, at least 800% higher, at least 900% higher, at least 1000% higher, or more, than the normal level of MECOM transcript expression.

In embodiments of the invention, the increased level of HMGA2 and/or MECOM is determined at about 1 week, about 2 weeks, about 3 weeks, about 4 weeks, about 5 weeks, about 6 weeks, about 7 weeks, about 8 weeks, about 9 weeks, about 10 weeks, about 11, weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1 week to about 2 weeks, about 2 weeks to about 3 weeks, 3 weeks to 4 weeks, 4 weeks to 5 weeks, 5 weeks to 6 weeks, 6 weeks to 7 weeks, 7 weeks to 8 weeks, 8 weeks to 9 weeks, 9 weeks to 10 weeks, 10 weeks to 11 weeks, 11 weeks to 12 weeks, 12 weeks to 16 weeks, 16 weeks to 20 weeks, 20 weeks to 24 weeks, 24 weeks to 28 weeks, 28 weeks to 32 weeks, 32 weeks to 36 weeks, 36 weeks to 40 weeks, 40 weeks to 44 weeks, 44 weeks to 48 weeks, 48 weeks to 52 weeks, and/or more than 52 weeks before administering an initial dose of an anti-PDL1/TGFβ Trap molecule.

In the methods of the present invention, a TNBC patient who has been determined to have an increased level of high mobility group AT-hook 2 (HMGA2) or MDS1 and EVI1 complex locus (MECOM) expression relative to a corresponding known control level, is administered a dose of at least 1200 mg of an anti-PD-L1/TGFβ Trap protein comprising a first polypeptide and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ). The second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, the method of treating TNBC or inhibiting tumor growth of the present disclosure involves administering to a subject who has been determined to have an increased level of high mobility group AT-hook 2 (HMGA2) or MDS1 and EVI1 complex locus (MECOM) expression relative to a corresponding known control level an anti-PD-L1/TGFβ Trap protein including two peptides in which the first polypeptide includes the amino acid sequence of SEQ ID NO: 3, and a second polypeptide includes the amino acid sequence of SEQ ID NO: 1.

In certain embodiments, the method of treating TNBC or inhibiting tumor growth of the present disclosure involves administering to a subject who has been determined to have an increased level of high mobility group AT-hook 2 (HMGA2) or MDS1 and EVI1 complex locus (MECOM) expression relative to a corresponding known control level, a protein (e.g., an anti-PD-L1/TGFβ Trap molecule (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40)) at a dose of about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg). In certain embodiments, about 1200 mg of anti-PD-L1/TGFβ Trap molecule is administered to a TNBC patient once every two weeks. In certain embodiments, about 1800 mg of anti-PD-L1/TGFβ Trap molecule is administered to a TNBC patient once every three weeks. In certain embodiments, about 2400 mg of anti-PD-L1/TGFβ Trap molecule is administered to a TNBC patient once every three weeks. In certain embodiments, about 1200 mg of a protein product with a first polypeptide that includes the amino acid sequence of SEQ ID NO: 3 and the second polypeptide that includes the amino acid sequence of SEQ ID NO: 1 is administered to a subject once every two weeks. In certain embodiments, about 1800 mg of a protein product with a first polypeptide that includes the amino acid sequence of SEQ ID NO: 3 and the second polypeptide that includes the amino acid sequence of SEQ ID NO: 1 is administered to a TNBC patient, who has been determined to have an increased level of high mobility group AT-hook 2 (HMGA2) or MDS1 and EVT1 complex locus (MECOM) expression relative to a corresponding known control level, once every three weeks. In certain embodiments, about 1800 mg of a protein product with a first polypeptide that comprises the amino acid sequences of SEQ TD NOs: 35, 36, and 37, and a second polypeptide that comprises the amino acid sequences of SEQ ID NOs: 38, 39, and 40 is administered to a TNBC patient once every three weeks. In certain embodiments, about 2400 mg of a protein product with a first polypeptide that includes the amino acid sequence of SEQ ID NO: 3 and the second polypeptide that includes the amino acid sequence of SEQ ID NO: 1 is administered to a TNBC patient, who has been determined to have an increased level of high mobility group AT-hook 2 (HMGA2) or MDS1 and EVI1 complex locus (MECOM) expression relative to a corresponding known control level, once every three weeks. In certain embodiments, about 2400 mg of a protein product with a first polypeptide that comprises the amino acid sequences of SEQ ID NOs: 35, 36, and 37, and a second polypeptide that comprises the amino acid sequences of SEQ ID NOs: 38, 39, and 40 is administered to a TNBC patient once every three weeks.

In certain embodiments, the dose administered to a TNBC patient may be about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, 1875 mg, about 1900 mg, about 1925 mg, about 1950 mg, about 1975 mg, about 2000 mg, about 2025 mg, about 2050 mg, about 2075 mg, 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, or about 2400 mg.

In certain embodiments, the subject who has been determined to have an increased level of HMGA2 or MECOM expression relative to a corresponding known control level is administered at least 1200 mg of the anti-PD-L1/TGFβ Trap protein. In certain embodiments, the subject who has been determined to have an increased level of HMGA2 or MECOM expression relative to a corresponding known control level is administered at least 1200 mg of the anti-PD-L1/TGFβ Trap protein. In certain embodiments, the subject who has been determined to have an increased level of HMGA2 or MECOM expression relative to a corresponding known control level is administered 1200 mg to 3000 mg of the anti-PD-L1/TGFβ Trap protein. In certain embodiments, the subject who has been determined to have an increased level of HMGA2 or MECOM expression relative to a corresponding known control level is administered 1200 mg to 2400 mg of the anti-PD-L1/TGFβ Trap protein. In certain embodiments, the subject who has been determined to have an increased level of HMGA2 or MECOM expression relative to a corresponding known control level is administered 1800 mg to 2400 mg of the anti-PD-L1/TGFβ Trap protein.

In certain embodiments, the subject who has been determined to have an increased level of HMGA2 or MECOM expression relative to a corresponding known control level is administered 1200 mg of the anti-PD-L1/TGFβ Trap protein, once every two weeks. In certain embodiments, the subject who has been determined to have an increased level of HMGA2 or MECOM expression relative to a corresponding known control level is administered 1800 mg of the anti-PD-L1/TGFβ Trap protein, once every three weeks. In certain embodiments, the subject who has been determined to have an increased level of HMGA2 or MECOM expression relative to a corresponding known control level is administered 2400 mg of the anti-PD-L1/TGFβ Trap protein, once every three weeks.

In certain embodiments, the subject who has been determined to have an increased level of HMGA2 or MECOM expression relative to a corresponding known control level is administered 2100 mg of the anti-PD-L1/TGFβ Trap protein. In certain embodiments, the subject who has been determined to have an increased level of HMGA2 or MECOM expression relative to a corresponding known control level is administered 2100 mg of the anti-PD-L1/TGFβ Trap protein, once every three weeks. In certain embodiments, the subject who has been determined to have an increased level of HMGA2 or MECOM expression relative to a corresponding known control level is administered 2400 mg or 3000 mg of the anti-PD-L1/TGFβ Trap protein, once every three weeks.

In certain embodiments, the dose administered to a TNBC patient may be administered once every two weeks. In certain embodiments, the dose administered to a TNBC patient may be administered once every three weeks. In certain embodiments, the protein may be administered by intravenous administration, e.g., with a prefilled bag, a prefilled pen, or a prefilled syringes. In certain embodiments, the protein is administered intravenously from a 250 ml saline bag, and the intravenous infusion may be for about one hour (e.g., 50 to 80 minutes). In certain embodiments, the bag is connected to a channel comprising a tube and/or a needle.

In certain embodiments, subjects or patients with TNBC who has been determined to have an increased level of high mobility group AT-hook 2 (HMGA2) or MDS1 and EVI1 complex locus (MECOM) expression relative to a corresponding known control level are treated by intravenously administering at least 1200 mg (e.g., about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more) of anti-PD-L1/TGFβ Trap, which includes a first polypeptide that includes the amino acid sequence of SEQ ID NO: 3, and a second polypeptide that includes the amino acid sequence of SEQ ID NO: 1. In certain embodiments, subjects or patients with TNBC (are treated by intravenously administering at least 1200 mg (e.g., about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more) of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40. In certain embodiments, subjects or patients with TNBC are treated by intravenously administering 1200 mg of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40. In certain embodiments, subjects or patients with TNBC are treated by intravenously administering 2400 mg of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40.

In certain embodiments, subjects or patients with TNBC who has been determined to have an increased level of high mobility group AT-hook 2 (HMGA2) or MDS1 and EVI1 complex locus (MECOM) expression relative to a corresponding known control level are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that includes the amino acid sequence of SEQ ID NO: 3, and a second polypeptide that includes the amino acid sequence of SEQ ID NO: 1.

In certain embodiments, subjects or patients with TNBC who has been determined to have an increased level of high mobility group AT-hook 2 (HMGA2) or MDS1 and EVI1 complex locus (MECOM) expression relative to a corresponding known control level are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40.

In some embodiments, subjects or patients with TNBC who has been determined to have an increased level of high mobility group AT-hook 2 (HMGA2) or MDS1 and EVI1 complex locus (MECOM) expression relative to a corresponding known control level are treated by intravenously administering anti-PD-L1/TGFβ Trap at a dose of about 1200 mg once every 2 weeks. In some embodiments, subjects or patients with advanced TNBC are treated by intravenously administering anti-PD-L1/TGFβ Trap at a dose of 1200 mg once every 2 weeks. In some embodiments, subjects or patients with advanced TNBC are treated by intravenously administering anti-PD-L1/TGFβ Trap at a dose of about 1800 mg once every 3 weeks. In some embodiments, subjects or patients with advanced TNBC are treated by intravenously administering anti-PD-L1/TGFβ Trap at a dose of about 2400 mg once every 3 weeks. In some embodiments, subjects or patients with advanced TNBC are treated by intravenously administering anti-PD-L1/TGFβ Trap at a dose of 2400 mg once every 3 weeks.

In certain embodiments, the TNBC to be treated is PD-L1 positive. For example, in certain embodiments, the TNBC to be treated exhibits PD-L1+ expression (e.g., high PD-L1 expression). In some embodiments for example, PD-L1 high may be defined as ≥80% PD-L1 positive tumor cells (tumor proportion score [TPS]) as determined by the 73-10 assay. In some embodiments, PD-L1 high may be defined as Tumor Proportion Score (TPS)≥50% as determined by the PD-L1 IHC 22C3 pharmDx assay. In some embodiments, PD-L1 high may be defined as Tumor Proportion Score (TPS)≥25% as determined by the PD-L1 IHC SP263 assay. Methods of detecting a biomarker, such as PD-L1 for example, on a cancer or tumor, are routine in the art and are contemplated herein. Non-limiting examples include immunohistochemistry, immunofluorescence and fluorescence activated cell sorting (FACS). In some embodiments, subjects or patients with PD-L1 high TNBC are treated by intravenously administering anti-PD-L1/TGFβ Trap at a dose of about 1200 mg once every 2 weeks. In some embodiments, subjects or patients with PD-L1 high TNBC are treated by intravenously administering anti-PD-L1/TGFβ Trap at a dose of about 1800 mg once every 3 weeks. In some embodiments, subjects or patients with PD-L1 high TNBC are treated by intravenously administering anti-PD-L1/TGFβ Trap at a dose of about 2100 mg once every 3 weeks. In some embodiments, subjects or patients with PD-L1 high TNBC are treated by intravenously administering anti-PD-L1/TGFβ Trap at a dose of about 2400 mg once every 3 weeks.

In some embodiments, the methods of treatment disclosed herein result in a disease response or improved survival of the subject or patient. In some embodiments for example, the disease response may be a complete response, a partial response, or a stable disease. In some embodiments for example, the improved survival could be progression-free survival (PFS) or overall survival. In some embodiments, improvement (e.g., in PFS) is determined relative to a period prior to initiation of treatment with an anti-PD-L1/TGFβ Trap of the present disclosure. Methods of determining disease response (e.g., complete response, partial response, or stable disease) and patient survival (e.g., PFS, overall survival) for cancer or tumor therapy are routine in the art and are contemplated herein. In some embodiments, disease response is evaluated according to RECIST 1.1 after subjecting the treated patient to contrast-enhanced computed tomography (CT) or magnetic resonance imaging (MRI) of the affected area.

### TGFβ as a Cancer Target

The current disclosure permits localized reduction in TGFβ in a tumor microenvironment by capturing the TGFβ using a soluble cytokine receptor (TGFβRII) tethered to an antibody moiety targeting a cellular immune checkpoint receptor found on the exterior surface of certain tumor cells or immune cells. An example of an antibody moiety of the disclosure to an immune checkpoint protein is anti-PD-L1. The bifunctional molecule of the present disclosure, sometimes referred to herein as an “antibody-cytokine Trap,” is effective precisely because the anti-receptor antibody and cytokine Trap are physically linked. The resulting advantage (over, for example, administration of the antibody and the receptor as separate molecules) is partly because cytokines function predominantly in the local environment through autocrine and paracrine functions. The antibody moiety directs the cytokine Trap to the tumor microenvironment where it can be most effective, by neutralizing the local immunosuppressive autocrine or paracrine effects. Furthermore, in cases where the target of the antibody is internalized upon antibody binding, an effective mechanism for clearance of the cytokine/cytokine receptor complex is provided. Antibody-mediated target internalization was shown for PD-L1, and anti-PD-L1/TGFβ Trap was shown to have a similar internalization rate as anti-PD-L1. This is a distinct advantage over using an anti-TGFβ antibody because first, an anti-TGFβ antibody might not be completely neutralizing; and second, the antibody can act as a carrier extending the half-life of the cytokine.

Indeed, as described below, treatment with the anti-PD-L1/TGFβ Trap elicits a synergistic anti-tumor effect due to the simultaneous blockade of the interaction between PD-L1 on tumor cells and PD-1 on immune cells, and the neutralization of TGFβ in the tumor microenvironment. Without being bound by theory, this presumably is due to a synergistic effect obtained from simultaneous blocking the two major immune escape mechanisms, and in addition, the depletion of the TGFβ in the tumor microenvironment by a single molecular entity. This depletion is achieved by (1) anti-PD-L1 targeting of tumor cells; (2) binding of the TGFβ autocrine/paracrine in the tumor microenvironment by the TGFβ Trap; and (3) destruction of the bound TGFβ through the PD-L1 receptor-mediated endocytosis. Furthermore, the TGFβRTT fused to the C-terminus of Fc (fragment of crystallization of IgG) was several-fold more potent than the TGFβRII-Fc that places the TGFβRII at the N-terminus of Fc.

TGFβ had been a somewhat questionable target in cancer immunotherapy because of its paradoxical roles as the molecular Jekyll and Hyde of cancer (Bierie et al., Nat. Rev. Cancer, 2006; 6:506-20). Like some other cytokines, TGFβ activity is developmental stage and context dependent. Indeed TGFβ can act as cither a tumor promoter or a tumor suppressor, affecting tumor initiation, progression and metastasis. The mechanisms underlying this dual role of TGFβ remain unclear (Yang et al., Trends Immunol. 2010; 31:220-227). Although it has been postulated that Smad-dependent signaling mediates the growth inhibition of TGFβ signaling, while the Smad independent pathways contribute to its tumor-promoting effect, there are also data showing that the Smad-dependent pathways are involved in tumor progression (Yang et al., Cancer Res. 2008; 68:9107-11).

Both the TGFβ ligand and the receptor have been studied intensively as therapeutic targets. There are three ligand isoforms, TGFβ1, 2 and 3, all of which exist as homodimers. There are also three TGFβ receptors (TGFβR), which are called TGFβR type I, II and III (Lopez-Casillas et al., J. Cell Biol. 1994; 124:557-68). TGFβRI is the signaling chain and cannot bind ligand. TGFβRII binds the ligand TGFβ1 and 3, but not TGFβ2, with high affinity. The TGFβRII/TGFβ complex recruits TGFβRI to form the signaling complex (Won et al., Cancer Res. 1999; 59:1273-7). TGFβRIII is a positive regulator of TGFβ binding to its signaling receptors and binds all 3 TGFβ isoforms with high affinity. On the cell surface, the TGFβ/TGFβRIII complex binds TGFβRII and then recruits TGFβRI, which displaces TGFβRIII to form the signaling complex.

Although the three different TGFβ isoforms all signal through the same receptor, they are known to have differential expression patterns and non-overlapping functions in vivo. The three different TGF-β isoform knockout mice have distinct phenotypes, indicating numerous non-compensated functions (Bujak et al., Cardiovasc. Res. 2007; 74:184-95). While TGFβ1 null mice have hematopoiesis and vasculogenesis defects and TGFβ3 null mice display pulmonary development and defective palatogenesis, TGFβ2 null mice show various developmental abnormalities, the most prominent being multiple cardiac deformities (Bartram et al., Circulation 2001; 103:2745-52; Yamagishi et al., Anat. Rec. 2012; 295:257-67). Furthermore, TGFβ is implicated to play a major role in the repair of myocardial damage after ischemia and reperfusion injury. In an adult heart, cardiomyocytes secrete TGFβ, which acts as an autocrine to maintain the spontaneous beating rate. Importantly, 70-85% of the TGFβ secreted by cardiomyocytes is TGFβ2 (Roberts et al., J. Clin. Invest. 1992; 90:2056-62). Despite cardiotoxicity concerns raised by treatment with TGFβRI kinase inhibitors, the present applicant has observed a lack of toxicity, including cardiotoxicity, for anti-PD-L1/TGFβ Trap in monkeys.

Therapeutic approaches to neutralize TGFβ include using the extracellular domains of TGFβ receptors as soluble receptor Traps and neutralizing antibodies. Of the receptor Trap approach, soluble TGFβRIII may seem the obvious choice since it binds all the three TGFβ ligands. However, TGFβRIII, which occurs naturally as a 280-330 kD glucosaminoglycan (GAG)-glycoprotein, with extracellular domain of 762 amino acid residues, is a very complex protein for biotherapeutic development. The soluble TGFβRIII devoid of GAG could be produced in insect cells and has been shown to be a potent TGFβ neutralizing agent (Vilchis-Landeros et al., Biochem. J., (2001), 355:215). The two separate binding domains (the endoglin-related and the uromodulin-related) of TGFβRIII could be independently expressed, but they were shown to have affinities 20 to 100 times lower than that of the soluble TGFβRIII, and much diminished neutralizing activity (Mendoza et al., Biochemistry 2009; 48:11755-65). On the other hand, the extracellular domain of TGFβRII is only 136 amino acid residues in length and can be produced as a glycosylated protein of 25-35 kD. The recombinant soluble TGFβRII was further shown to bind TGFβ1 with a K_{D} of 200 pM, which is fairly similar to the K_{D} of 50 pM for the full length TGFβRII on cells (Lin et al., J. Biol. Chem. 1995; 270:2747-54). Soluble TGFβRII-Fc was tested as an anti-cancer agent and was shown to inhibit established murine malignant mesothelioma growth in a tumor model (Suzuki et al., Clin. Cancer Res., 2004; 10:5907-18). Because TGFβRII does not bind TGFβ2, and TGFβRIII binds TGFβ1 and 3 with lower affinity than TGFβRII, a fusion protein of the endoglin domain of TGFβRIII and extracellular domain of TGFβRII was produced in bacteria and was shown to inhibit the signaling of TGFβ1 and 2 in cell based assays more effectively than either TGFβRII or RIII (Verona et al., Protein Eng'g. Des. Sel. 2008; 21:463-73).

Still another approach to neutralize all three isoforms of the TGFβ ligands is to screen for a pan-neutralizing anti-TGFβ antibody, or an anti-receptor antibody that blocks the receptor from binding to TGFβ1, 2 and 3. GC1008, a human antibody specific for all isoforms of TGFβ, was in a Phase I/II study in patients with advanced malignant melanoma or renal cell carcinoma (Morris et al., J. Clin. Oncol. 2008; 26:9028 (Meeting abstract)). Although the treatment was found to be safe and well tolerated, only limited clinical efficacy was observed, and hence it was difficult to interpret the importance of anti-TGFβ therapy without further characterization of the immunological effects (Flavell et al., Nat. Rev. Immunol. 2010; 10:554-67). There were also TGFβ-isoform-specific antibodies tested in the clinic. Metelimumab, an antibody specific for TGFβ1, was tested in Phase 2 clinical trial as a treatment to prevent excessive post-operative scarring for glaucoma surgery; and Lerdelimumab, an antibody specific for TGFβ2, was found to be safe but ineffective at improving scarring after eye surgery in a Phase 3 study (Khaw et al., Ophthalmology 2007; 114:1822-1830). Anti-TGFβRII antibodies that block the receptor from binding to all the three TGFβ isoforms, such as the anti-human TGFβRII antibody TR1 and anti-mouse TGFβRII antibody MT1, have also shown some therapeutic efficacy against primary tumor growth and metastasis in mouse models (Zhong et al., Clin. Cancer Res. 2010; 16:1191-205). However, in a recent Phase I study of antibody TR1 (LY3022859), dose escalation beyond 25 mg (flat dose) was considered unsafe due to uncontrolled cytokine release, despite prophylactic treatment (Tolcher et al., Cancer Chemother. Pharmacol. 2017; 79:673-680). To date, the vast majority of the studies on TGFβ targeted anticancer treatment, including small molecule inhibitors of TGFβ signaling that often are quite toxic, are mostly in the preclinical stage and the anti-tumor efficacy obtained has been limited (Calone et al., Exp Oncol. 2012; 34:9-16; Connolly et al., Int. J. Biol. Sci. 2012; 8:964-78).

The antibody-TGFβ Trap of the disclosure is a bifunctional protein containing at least portion of a human TGFβ Receptor II (TGFβRII) that is capable of binding TGFβ. In certain embodiments, the TGFβ Trap polypeptide is a soluble portion of the human TGFβ Receptor Type 2 Isoform A (SEQ ID NO: 8) that is capable of binding TGFβ. In certain embodiments, TGFβ Trap polypeptide contains at least amino acids 73-184 of SEQ ID NO: 8. In certain embodiments, the TGFβ Trap polypeptide contains amino acids 24-184 of SEQ ID NO: 8. In certain embodiments, the TGFβ Trap polypeptide is a soluble portion of the human TGFβ Receptor Type 2 Isoform B (SEQ ID NO: 9) that is capable of binding TGFβ. In certain embodiments, TGFβ Trap polypeptide contains at least amino acids 48-159 of SEQ ID NO: 9. In certain embodiments, the TGFβ Trap polypeptide contains amino acids 24-159 of SEQ ID NO: 9. In certain embodiments, the TGFβ Trap polypeptide contains amino acids 24-105 of SEQ ID NO: 9. In certain exemplary embodiments, the TGFβ Trap polypeptide contains the sequence of SEQ ID NOs: 10, 50, 51, 52, 53, or 54.

In another embodiment, the antibody-TGFβ Trap of the disclosure is one of the fusion proteins disclosed in WO 2018/205985. In some embodiments, the fusion protein is one of the constructs listed in Table 2 of this publication, such as construct 9 or 15 thereof. In other embodiments, the antibody having the heavy chain sequence of SEQ ID NO: 11 and the light chain sequence of SEQ ID NO: 12 of this publication [corresponding to SEQ ID NO: 61 and 62, respectively, of the present disclosure] is fused via a linking sequence (G₄S)ₓG, wherein x is 4-5, to the TGFβRII extracellular domain sequence of SEQ ID NO: 14 or SEQ ID NO: 15 of said publication [corresponding to SEQ ID NO: 50 and 51, respectively, of the present disclosure].

### Mechanisms of Action

The approach of targeting T cell inhibition checkpoints for dis-inhibition with therapeutic antibodies is an area of intense investigation (for a review, see Pardoll, Nat. Rev. Cancer 2012, 12:253-264). In one approach, the antibody moiety or antigen binding fragment thereof targets T cell inhibition checkpoint receptor proteins on the T cell, such as, for example: CTLA-4, PD-1, BTLA, LAG-3, TIM-3, or LAIR1. In another approach, the antibody moiety targets the counter-receptors on antigen presenting cells and tumor cells (which co-opt some of these counter-receptors for their own immune evasion), such as for example: PD-L1 (B7-H1), B7-DC, HVEM, TIM-4, B7-H3, or B7-H4.

The disclosure contemplates antibody TGFβ Traps that target, through their antibody moiety or antigen binding fragment thereof, T cell inhibition checkpoints for dis-inhibition. To that end the applicants have tested the anti-tumor efficacy of combining a TGFβ Trap with antibodies targeting various T cell inhibition checkpoint receptor proteins, such as anti-PD-1, anti-PD-L1, anti-TIM-3 and anti-LAG3.

The programmed death 1 (PD-1)/PD-L1 axis is an important mechanism for tumor immune evasion. Effector T cells chronically sensing antigen take on an exhausted phenotype marked by PD-1 expression, a state under which tumor cells engage by upregulating PD-L1. Additionally, in the tumor microenvironment, myeloid cells, macrophages, parenchymal cells and T cells upregulate PD-L1. Blocking the axis restores the effector function in these T cells. Anti-PD-L1/TGFβ Trap also binds TGFβ (1, 2, and 3 isoforms), which is an inhibitory cytokine produced in the tumor microenvironment by cells including apoptotic neutrophils, myeloid-derived suppressor cells, T cells and tumor. Inhibition of TGFβ by soluble TGFβRII reduced malignant mesothelioma in a manner that was associated with increases in CD8+ T cell anti-tumor effects. The absence of TGFβ1 produced by activated CD4+ T cells and Treg cells has been shown to inhibit tumor growth and protect mice from spontaneous cancer. Thus, TGFβ appears to be important for tumor immune evasion.

TGFβ has growth inhibitory effects on normal epithelial cells, functioning as a regulator of epithelial cell homeostasis, and it acts as a tumor suppressor during early carcinogenesis. As tumors progress toward malignancy, the growth inhibitory effects of TGFβ on the tumor are lost via mutation in one or more TGFβ pathway signaling components or through oncogenic reprogramming. Upon loss of sensitivity to TGFβ inhibition, the tumor continues to produce high levels of TGFβ, which then serve to promote tumor growth. The TGFβ cytokine is overexpressed in various cancer types with correlation to tumor stage. Many types of cells in the tumor microenvironment produce TGFβ including the tumor cells themselves, immature myeloid cells, regulatory T cells, and stromal fibroblasts; these cells collectively generate a large reservoir of TGFβ in the extracellular matrix. TGFβ signaling contributes to tumor progression by promoting metastasis, stimulating angiogenesis, and suppressing innate and adaptive anti-tumor immunity. As a broadly immunosuppressive factor, TGFβ directly down-regulates the effector function of activated cytotoxic T cells and NK cells and potently induces the differentiation of naïve CD4+ T cells to the immunosuppressive regulatory T cells (Treg) phenotype. In addition, TGFβ polarizes macrophages and neutrophils to a wound-healing phenotype that is associated with production of immunosuppressive cytokines. As a therapeutic strategy, neutralization of TGFβ activity has the potential to control tumor growth by restoring effective anti-tumor immunity, blocking metastasis, and inhibiting angiogenesis.

The present disclosure provides dosage regimens for targeted TGF-β inhibition with an anti-PD-L1/TGFβ Trap molecule for use in a method of treating a subject diagnosed with TNBC.

Concomitant PD-1 and TGFβ blockade can restore pro-inflammatory cytokines. Anti-PD-L1/TGFβ Trap includes, for example, an extracellular domain of the human TGFβ receptor TGFβRII covalently joined via a glycine/serine linker to the C-terminus of each heavy chain of the fully human IgG1 anti-PD-L1 antibody. Given the emerging picture for the anti-PD-1/PD-L1 class, in which responses are apparent but with room for increase in effect size, it is assumed that co-targeting a complementary immune modulation step will improve tumor response. A similar TGF-targeting agent, fresolimumab, which is a monoclonal antibody targeting TGFβ1, 2 and 3, showed initial evidence of tumor response in a Phase I trial in subjects with melanoma.

### Anti-PD-L1 Antibodies

The anti-PD-L1/TGFβ Trap molecule of the present disclosure can include any anti-PD-L1 antibody, or antigen-binding fragment thereof, described in the art. Anti-PD-L1 antibodies are commercially available, for example, the 29E2A3 antibody (Biolegend, Cat. No. 329701). Antibodies can be monoclonal, chimeric, humanized, or human. Antibody fragments include Fab, F(ab′)2, scFv and Fv fragments, which are described in further detail below.

Exemplary antibodies are described in PCT Publication WO 2013/079174. These antibodies can include a heavy chain variable region polypeptide including an HVR-H1, HVR-H2, and HVR-H3 sequence, where:

(a) the HVR-H1 sequence is
(SEQ ID NO: 21)
X₁YX₂MX₃;
(b) the HVR-H2 sequence is
(SEQ ID NO: 22)
SIYPSGGX₄TFYADX₅VKG;
(c) the HVR-H3 sequence is
(SEQ ID NO: 23)
IKLGTVTTVX₆Y;

further where: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D.

In a one embodiment, X₁ is M, I, or S; X₂ is R, K, L, M, or I; X₃ is F or M; X₄ is F or I; X₅ is S or T; X₆ is E or D.

In another embodiment X₁ is M, I, or S; X₂ is L, M, or I; X₃ is F or M; X₄ is I; X₅ is S or T; X₆ is D.

In still another embodiment, X₁ is S; X₂ is I; X₃ is M; X₄ is I; X₅ is T; X₆ is D.

In another aspect, the polypeptide further includes variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4).

In yet another aspect, the framework sequences are derived from human consensus framework sequences or human germline framework sequences.

In a still further aspect, at least one of the framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the heavy chain polypeptide is further combined with a variable region light chain including an HVR-L1, HVR-L2, and HVR-L3, where:

(a) the HVR-L1 sequence is
(SEQ ID NO: 28)
TGTX₇X₈DVGX₉YNYVS;
(b) the HVR-L2 sequence is
(SEQ ID NO: 29)
X₁₀VX₁₁X₁₂RPS;
(c) the HVR-L3 sequence is
(SEQ ID NO: 30)
SSX₁₃TX₁₄X₁₅X₁₆X₁₇RV;

further where: X₇ is N or S; X₈ is T, R, or S; X₉ is A or G; X₁₀ is E or D; X₁₁ is I, N or S; X₁₂ is D, H or N; X₁₃ is F or Y; X₁₄ is N or S; X₁₅ is R, T or S; X₁₆ is G or S; X₁₇ is I or T.

In another embodiment, X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a still further aspect, the light chain further includes variable region light chain framework sequences juxtaposed between the HVRs according to the formula: (LC-FR1MHVR-L1)-(LC-FR2)-(HVR-L2)-(LC-FR3)-(HVR-L3)-(LC-FR4).

In a still further aspect, the light chain framework sequences are derived from human consensus framework sequences or human germline framework sequences.

In a still further aspect, the light chain framework sequences are lambda light chain sequences.

In a still further aspect, at least one of the framework sequence is the following:

LC-FR1 is
(SEQ ID NO: 31)
QSALTQPASVSGSPGQSITISC;
LC-FR2 is
(SEQ ID NO: 32)
WYQQHPGKAPKLMIY;
LC-FR3 is
(SEQ ID NO: 33)
GVSNRFSGSKSGNTASLTISGLQAEDEADYYC;
LC-FR4 is
(SEQ ID NO: 34)
FGTGTKVTVL.

In another embodiment, the disclosure provides an anti-PD-L1 antibody or antigen binding fragment including a heavy chain and a light chain variable region sequence, where:

(a) the heavy chain includes an HVR-H1, HVR-H2, and HVR-H3, wherein further: (i) the HVR-H1 sequence is X₁YX₂MX₃ (SEQ ID NO: 21); (ii) the HVR-H2 sequence is SIYPSGGX₄TFYADX₅VKG (SEQ ID NO: 22); (iii) the HVR-H3 sequence is IKLGTVTTVX₆Y (SEQ ID NO: 23), and;

(b) the light chain includes an HVR-L1, HVR-L2, and HVR-L3, wherein further: (iv) the HVR-L1 sequence is TGTX₇X₈DVGX₉YNYVS (SEQ TD NO: 28); (v) the HVR-L2 sequence is X₁₀VX₁₁X₁₂RPS (SEQ ID NO: 29); (vi) the HVR-L3 sequence is SSX₁₃TX₁₄X₁₅X₁₆X₁₇RV (SEQ ID NO: 30); wherein: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D; X₇ is N or S; X₈ is T, R, or S; X₉ is A or G; X₁₀ is E or D; X₁₁ is I, N, or S; X₁₂ is D, H, or N; X₁₃ is F or Y; X₁₄ is N or S; X₁₅ is R, T, or S; X₁₆ is G or S; X₁₇ is I or T.

In one embodiment, X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In another embodiment, X₁ is M, I, or S; X₂ is L, M, or I; X₃ is F or M; X₄ is I; X₅ is S or T; X₆ is D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₁ is S; X₂ is I; X₃ is M; X₄ is I; X₅ is T; X₆ is D; X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a further aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1 MHVR-L1)-(LC-FR2)-(HVR-L2)-(LC-FR3)-(HVR-L3)-(LC-FR4).

In a still further aspect, the framework sequences are derived from human consensus framework sequences or human germline sequences.

In a still further aspect, one or more of the heavy chain framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the light chain framework sequences are lambda light chain sequences.

In a still further aspect, one or more of the light chain framework sequences is the following:

LC-FR1 is
(SEQ ID NO: 31)
QSALTQPASVSGSPGQSITISC;
LC-FR2 is
(SEQ ID NO: 32)
WYQQHPGKAPKLMIY;
LC-FR3 is
(SEQ ID NO: 33)
GVSNRFSGSKSGNTASLTISGLQAEDEADYYC;
LC-FR4 is
(SEQ ID NO: 34)
FGTGTKVTVL.

In a still further aspect, the heavy chain variable region polypeptide, antibody, or antibody fragment further includes at least a C_{H}1 domain.

In a more specific aspect, the heavy chain variable region polypeptide, antibody, or antibody fragment further includes a C_{H}1, a C_{H}2, and a C_{H}3 domain.

In a still further aspect, the variable region light chain, antibody, or antibody fragment further includes a C_{L} domain.

In a still further aspect, the antibody further includes a C_{H}1, a C_{H}2, a C_{H}3, and a C_{L} domain.

In a still further specific aspect, the antibody further includes a human or murine constant region.

In a still further aspect, the human constant region is selected from the group consisting of IgG1, IgG2, IgG2, IgG3, IgG4.

In a still further specific aspect, the human or murine constant region is IgG1.

In yet another embodiment, the disclosure features an anti-PD-L1 antibody including a heavy chain and a light chain variable region sequence, where:

(a) the heavy chain includes an HVR-H1, an HVR-H2, and an HVR-H3, having at least 80% overall sequence identity to SYIMM (SEQ ID NO: 35), SIYPSGGITFYADTVKG (SEQ ID NO: 36), and IKLGTVTTVDY (SEQ ID NO: 37), respectively, and

(b) the light chain includes an HVR-L1, an HVR-L2, and an HVR-L3, having at least 80% overall sequence identity to TGTSSDVGGYNYVS (SEQ ID NO: 38), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In yet another embodiment, the disclosure features an anti-PD-L1 antibody including a heavy chain and a light chain variable region sequence, where:

(a) the heavy chain includes an HVR-H1, an HVR-H2, and an HVR-H3, having at least 80% overall sequence identity to MYMMM (SEQ ID NO: 41), SIYPSGGITFYADSVKG (SEQ ID NO: 42), and IKLGTVTTVDY (SEQ ID NO: 37), respectively, and

(b) the light chain includes an HVR-L1, an HVR-L2, and an HVR-L3, having at least 80% overall sequence identity to TGTSSDVGAYNYVS (SEQ ID NO: 43), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a still further aspect, in the antibody or antibody fragment according to the disclosure, as compared to the sequences of HVR-H1, HVR-H2, and HVR-H3, at least those amino acids remain unchanged that are highlighted by underlining as follows:

(a) in HVR-H1
(SEQ ID NO: 35)
SYIMM,
(b) in HVR-H2
(SEQ ID NO: 36)
SIYPSGGITFYADTVKG,
(c) in HVR-H3
(SEQ ID NO: 37)
IKLGTVTTVDY;

and further where, as compared to the sequences of HVR-L1, HVR-L2, and HVR-L3 at least those amino acids remain unchanged that are highlighted by underlining as follows:

(a) HVR-L1
(SEQ ID NO: 38)
TGTSSDVGGYNYVS
(b) HVR-L2
(SEQ ID NO: 39)
DVSNRPS
(c) HVR-L3
(SEQ ID NO: 40)
SSYTSSSTRV.

In another aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1)-(HVR-L1)-(LC-FR2)-(HVR-L2)-(LC-FR3)-(HVR-L3)-(LC-FR4).

In yet another aspect, the framework sequences are derived from human germline sequences.

In a still further aspect, one or more of the heavy chain framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the light chain framework sequences are derived from a lambda light chain sequence.

In a still further aspect, one or more of the light chain framework sequences is the following:

LC-FR1 is
(SEQ ID NO: 31)
QSALTQPASVSGSPGQSITISC;
LC-FR2 is
(SEQ ID NO: 32)
WYQQHPGKAPKLMIY;
LC-FR3 is
(SEQ ID NO: 33)
GVSNRFSGSKSGNTASLTISGLQAEDEADYYC;
LC-FR4 is
(SEQ ID NO: 34)
FGTGTKVTVL.

In a still further specific aspect, the antibody further includes a human or murine constant region.

In a still further aspect, the human constant region is selected from the group consisting of IgG1, IgG2, IgG2, IgG3, IgG4.

In certain embodiments, the disclosure features an anti-PD-L1 antibody including a heavy chain and a light chain variable region sequence, where:

(a) the heavy chain sequence has at least 85% sequence identity to the heavy chain sequence:

(SEQ ID NO: 44)
EVQLLESGGGLVQPGGSLRLSCAASGFTFSSYIMMVWRQAPGKGLEW
VSSIYPSGGITFYADWKGRFTISRDNSKNTLYLQMNSLRAEDTAVYY
CARIKLGTVTTVDYWGQGTLVTVSS,

and

(b) the light chain sequence has at least 85% sequence identity to the light chain sequence:

(SEQ ID NO: 45)
QSALTQPASVSGSPGQSITISCTGTSSDVGGYNYVSWYQQHPGKAPK
LMIYDVSNRPSGVSNRFSGSKSGNTASLTISGLQAEDEADYYCSSYT
SSSTRVFGTGTKVTVL.

In various embodiments, the heavy chain sequence has at least 86% sequence identity to SEQ ID NO: 44 and the light chain sequence has at least 86% sequence identity to SEQ ID NO: 45; the heavy chain sequence has at least 87% sequence identity to SEQ ID NO: 44 and the light chain sequence has at least 87% sequence identity to SEQ ID NO: 45; the heavy chain sequence has at least 88% sequence identity to SEQ ID NO: 44 and the light chain sequence has at least 88% sequence identity to SEQ ID NO: 45; the heavy chain sequence has at least 89% sequence identity to SEQ ID NO: 44 and the light chain sequence has at least 89% sequence identity to SEQ ID NO: 45; the heavy chain sequence has at least, 90% sequence identity to SEQ ID NO: 44 and the light chain sequence has at least 90% sequence identity to SEQ ID NO: 45; the heavy chain sequence has at least 91% sequence identity to SEQ ID NO: 44 and the light chain sequence has at least 91% sequence identity to SEQ ID NO: 45; the heavy chain sequence has at least 92% sequence identity to SEQ ID NO: 44 and the light chain sequence has at least 92% sequence identity to SEQ ID NO: 45; the heavy chain sequence has at least 93% sequence identity to SEQ ID NO: 44 and the light chain sequence has at least 93% sequence identity to SEQ TD NO: 45; the heavy chain sequence has at least 94% sequence identity to SEQ ID NO: 44 and the light chain sequence has at least 94% sequence identity to SEQ ID NO: 45; the heavy chain sequence has at least 95% sequence identity to SEQ ID NO: 44 and the light chain sequence has at least 95% sequence identity to SEQ ID NO: 45; the heavy chain sequence has at least 96% sequence identity to SEQ ID NO: 44 and the light chain sequence has at least 96% sequence identity to SEQ ID NO: 45; the heavy chain sequence has at least 97% sequence identity to SEQ ID NO: 44 and the light chain sequence has at least 97% sequence identity to SEQ ID NO: 45; the heavy chain sequence has at least 98% sequence identity to SEQ ID NO: 44 and the light chain sequence has at least 98% sequence identity to SEQ ID NO: 45; the heavy chain sequence has at least 99% sequence identity to SEQ ID NO: 44 and the light chain sequence has at least 99% sequence identity to SEQ ID NO: 45; or the heavy chain sequence comprises SEQ ID NO: 44 and the light chain sequence comprises SEQ ID NO: 45.

In certain embodiments, the disclosure provides for an anti-PD-L1 antibody including a heavy chain and a light chain variable region sequence, where:

(a) the heavy chain sequence has at least 85% sequence identity to the heavy chain sequence:

(SEQ ID NO: 46)
EVQLLESGGGLVQPGGSLRLSCAASGFTFSMYMMMWVRQAPGKGLEV
WSSIYPSGGITFYADSVKGRFTISRDNSKNTLYLQMNSLRAEDTAIY
YCARIKLGTVTTVDYWGQGTLVTVSS,

and

(b) the light chain sequence has at least 85% sequence identity to the light chain sequence:

(SEQ ID NO: 47)
QSALTQPASVSGSPGQSITISCTGTSSDVGAYNYVSWYQQHPGKAPK
LMIYDVSNRPSGVSNRFSGSKSGNTASLTISGLQAEDEADYYCSSYT
SSSTRVFGTGTKVTVL.

In various embodiments, the heavy chain sequence has at least 86% sequence identity to SEQ TD NO: 46 and the light chain sequence has at least 86% sequence identity to SEQ ID NO: 47; the heavy chain sequence has at least 87% sequence identity to SEQ ID NO: 46 and the light chain sequence has at least 87% sequence identity to SEQ ID NO: 47; the heavy chain sequence has at least 88% sequence identity to SEQ ID NO: 46 and the light chain sequence has at least 88% sequence identity to SEQ ID NO: 47; the heavy chain sequence has at least 89% sequence identity to SEQ ID NO: 46 and the light chain sequence has at least 89% sequence identity to SEQ ID NO: 47; the heavy chain sequence has at least, 90% sequence identity to SEQ ID NO: 46 and the light chain sequence has at least 90% sequence identity to SEQ ID NO: 47; the heavy chain sequence has at least 91% sequence identity to SEQ ID NO: 46 and the light chain sequence has at least 91% sequence identity to SEQ ID NO: 47; the heavy chain sequence has at least 92% sequence identity to SEQ ID NO: 46 and the light chain sequence has at least 92% sequence identity to SEQ ID NO: 47; the heavy chain sequence has at least 93% sequence identity to SEQ ID NO: 46 and the light chain sequence has at least 93% sequence identity to SEQ ID NO: 47; the heavy chain sequence has at least 94% sequence identity to SEQ ID NO: 46 and the light chain sequence has at least 94% sequence identity to SEQ ID NO: 47; the heavy chain sequence has at least 95% sequence identity to SEQ ID NO: 46 and the light chain sequence has at least 95% sequence identity to SEQ ID NO: 47; the heavy chain sequence has at least 96% sequence identity to SEQ ID NO: 46 and the light chain sequence has at least 96% sequence identity to SEQ ID NO: 47; the heavy chain sequence has at least 97% sequence identity to SEQ ID NO: 46 and the light chain sequence has at least 97% sequence identity to SEQ ID NO: 47; the heavy chain sequence has at least 98% sequence identity to SEQ ID NO: 46 and the light chain sequence has at least 98% sequence identity to SEQ ID NO: 47; the heavy chain sequence has at least 99% sequence identity to SEQ ID NO: 46 and the light chain sequence has at least 99% sequence identity to SEQ TD NO: 47; or the heavy chain sequence comprises SEQ ID NO: 46 and the light chain sequence comprises SEQ ID NO: 47.

In another embodiment the antibody binds to human, mouse, or cynomolgus monkey PD-L1. In a specific aspect the antibody is capable of blocking the interaction between human, mice, or cynomolgus monkey PD-L1 and the respective human, mouse, or cynomolgus monkey PD-1 receptors.

In another embodiment, the antibody binds to human PD-L1 with a KD of 5×10⁻⁹ M or less, preferably with a KD of 2×10⁻⁹ M or less, and even more preferred with a KD of 1×10⁻⁹ M or less.

In yet another embodiment, the disclosure relates to an anti-PD-L1 antibody or antigen binding fragment thereof which binds to a functional epitope including residues Y56 and D61 of human PD-L1.

In a specific aspect, the functional epitope further includes E58, E60, Q66, R113, and M115 of human PD-L1.

In a more specific aspect, the antibody binds to a conformational epitope, including residues 54-66 and 112-122 of human PD-L1.

In certain embodiments, the disclosure is related to an anti-PD-L1 antibody, or antigen binding fragment thereof, which cross-competes for binding to PD-L1 with an antibody according to the disclosure as described herein.

In certain embodiments, the disclosure features proteins and polypeptides including any of the above described anti-PD-L1 antibodies in combination with at least one pharmaceutically acceptable carrier.

In certain embodiments, the disclosure features an isolated nucleic acid encoding a polypeptide, or light chain or a heavy chain variable region sequence of an anti-PD-L1 antibody, or antigen binding fragment thereof, as described herein. In certain embodiments, the disclosure provides for an isolated nucleic acid encoding a light chain or a heavy chain variable region sequence of an anti-PD-L1 antibody, wherein:

(a) the heavy chain includes an HVR-H1, an HVR-H2, and an HVR-H3 sequence having at least 80% sequence identity to SYIMM (SEQ ID NO: 35), SIYPSGGITFYADTVKG (SEQ ID NO: 36), and IKLGTVTTVDY (SEQ ID NO: 37), respectively, or

(b) the light chain includes an HVR-L1, an HVR-L2, and an HVR-L3 sequence having at least 80% sequence identity to TGTSSDVGGYNYVS (SEQ ID NO: 38), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a further aspect, the nucleic acid sequence for the heavy chain is:

| (SEQ ID NO: 48) | |
|---|---|
| atggagttgc ctgttaggct gttggtgctg atgttctgga ttcctgctag ctccagcgag | 60 |
| gtgcagctgc tggaatccgg cggaggactg gtgcagcctg gcggctccct gagactgtct | 120 |
| tgcgccgcct ccggcttcac cttctccagc tacatcatga tgtgggtgcg acaggcccct | 180 |
| ggcaagggcc tggaatgggt gtcctccatc tacccctccg gcggcatcac cttctacgcc | 240 |
| gacaccgtga agggccggtt caccatctcc cgggacaact ccaagaacac cctgtacctg | 300 |
| cagatgaact ccctgcgggc cgaggacacc gccgtgtact actgcgcccg gatcaagctg | 360 |
| ggcaccgtga ccaccgtgga ctactggggc cagggcaccc tggtgacagt gtcctccgcc | 420 |
| tccaccaagg gcccatcggt cttccccctg gcaccctcct ccaagagcac ctctgggggc | 480 |
| acagcggccc tgggctgcct ggtcaaggac tacttccccg aaccggtgac ggtgtcgtgg | 540 |
| aactcaggcg ccctgaccag cggcgtgcac accttcccgg ctgtcctaca gtcctcagga | 600 |
| ctctactccc tcagcagcgt ggtgaccgtg ccctccagca gcttgggcac ccagacctac | 660 |
| atctgcaacg tgaatcacaa gcccagcaac accaaggtgg acaagaaagt tgagcccaaa | 720 |
| tcttgtgaca aaactcacac atgcccaccg tgcccagcac ctgaactcct ggggggaccg | 780 |
| tcagtcttcc tcttcccccc aaaacccaag gacaccctca tgatctcccg gacccctgag | 840 |
| gtcacatgcg tggtggtgga cgtgagccac gaagaccctg aggtcaagtt caactggtac | 900 |
| gtggacggcg tggaggtgca taatgccaag acaaagccgc gggaggagca gtacaacagc | 960 |
| acgtaccgtg tggtcagcgt cctcaccgtc ctgcaccagg actggctgaa tggcaaggag | 1020 |
| tacaagtgca aggtctccaa caaagccctc ccagccccca tcgagaaaac catctccaaa | 1080 |
| gccaaagggc agccccgaga accacaggtg tacaccctgc ccccatcacg ggatgagctg | 1140 |
| accaagaacc aggtcagcct gacctgcctg gtcaaaggct tctatcccag cgacatcgcc | 1200 |
| gtggagtggg agagcaatgg gcagccggag aacaactaca agaccacgcc tcccgtgctg | 1260 |
| gactccgacg gctccttctt cctctatagc aagctcaccg tggacaagag caggtggcag | 1320 |
| caggggaacg tcttctcatg ctccgtgatg catgaggctc tgcacaacca ctacacgcag | 1380 |
| aagagcctct ccctgtcccc gggtaaa | 1407 |

and the nucleic acid sequence for the light chain is: PGP 35

| (SEQ ID NO: 49) | |
|---|---|
| atggagttgc ctgttaggct gttggtgctg atgttctgga ttcctgcttc cttaagccag | 60 |
| tccgccctga cccagcctgc ctccgtatct ggctcccctg gccagtccat caccatcagc | 120 |
| tgcaccggca cctccagcga cgtgggcggc tacaactacg tgtcctggta tcagcagcac | 180 |
| cccggcaagg cccccaagct gatgatctac gacgtgtcca accggccctc cggcgtgtcc | 240 |
| aacagattct ccggctccaa gtccggcaac accgcctccc tgaccatcag cggactgcag | 300 |
| gcagaggacg aggccgacta ctactgctcc tcctacacct cctccagcac cagagtgttc | 360 |
| ggcaccggca caaaagtgac cgtgctgggc cagcccaagg ccaacccaac cgtgacactg | 420 |
| ttccccccat cctccgagga actgcaggcc aacaaggcca ccctggtctg cctgatctca | 480 |
| gatttctatc caggcgccgt gaccgtggcc tggaaggctg atggctcccc agtgaaggcc | 540 |
| ggcgtggaaa ccaccaagcc ctccaagcag tccaacaaca aatacgccgc ctcctcctac | 600 |
| ctgtccctga cccccgagca gtggaagtcc caccggtcct acagctgcca ggtcacacac | 660 |
| gagggctcca ccgtggaaaa gaccgtcgcc cccaccgagt gctca. | 705 |

Further exemplary anti-PD-L1 antibodies that can be used in an anti-PD-L1/TGFβ Trap are described in US patent application publication US 2010/0203056. In one embodiment of the disclosure, the antibody moiety is YW243.55S70. In another embodiment of the disclosure, the antibody moiety is MPDL3289A.

In certain embodiments, the disclosure features an anti-PD-L1 antibody moiety including a heavy chain and a light chain variable region sequence, where:

(a) the heavy chain sequence has at least 85% sequence identity to the heavy chain sequence:

(SEQ ID NO: 12)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and

(b) the light chain sequence has at least 85% sequence identity to the light chain sequence:

(SEQ ID NO: 13)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In various embodiments, the heavy chain sequence has at least 86% sequence identity to SEQ ID NO: 12 and the light chain sequence has at least 86% sequence identity to SEQ ID NO: 13; the heavy chain sequence has at least 87% sequence identity to SEQ ID NO: 12 and the light chain sequence has at least 87% sequence identity to SEQ ID NO: 13; the heavy chain sequence has at least 88% sequence identity to SEQ ID NO: 12 and the light chain sequence has at least 88% sequence identity to SEQ ID NO: 13; the heavy chain sequence has at least 89% sequence identity to SEQ ID NO: 12 and the light chain sequence has at least 89% sequence identity to SEQ ID NO: 13; the heavy chain sequence has at least, 90% sequence identity to SEQ ID NO: 12 and the light chain sequence has at least 90% sequence identity to SEQ ID NO: 13; the heavy chain sequence has at least 91% sequence identity to SEQ ID NO: 12 and the light chain sequence has at least 91% sequence identity to SEQ ID NO: 13; the heavy chain sequence has at least 92% sequence identity to SEQ ID NO: 12 and the light chain sequence has at least 92% sequence identity to SEQ ID NO: 13; the heavy chain sequence has at least 93% sequence identity to SEQ ID NO: 12 and the light chain sequence has at least 93% sequence identity to SEQ ID NO: 13; the heavy chain sequence has at least 94% sequence identity to SEQ TD NO: 12 and the light chain sequence has at least 94% sequence identity to SEQ ID NO: 13; the heavy chain sequence has at least 95% sequence identity to SEQ ID NO: 12 and the light chain sequence has at least 95% sequence identity to SEQ ID NO: 13; the heavy chain sequence has at least 96% sequence identity to SEQ TD NO: 12 and the light chain sequence has at least 96% sequence identity to SEQ ID NO: 13; the heavy chain sequence has at least 97% sequence identity to SEQ ID NO: 12 and the light chain sequence has at least 97% sequence identity to SEQ ID NO: 13; the heavy chain sequence has at least 98% sequence identity to SEQ ID NO: 12 and the light chain sequence has at least 98% sequence identity to SEQ ID NO: 13; the heavy chain sequence has at least 99% sequence identity to SEQ ID NO: 12 and the light chain sequence has at least 99% sequence identity to SEQ ID NO: 13; or the heavy chain sequence comprises SEQ ID NO: 12 and the light chain sequence comprises SEQ ID NO: 13.

In certain embodiments, the disclosure features an anti-PD-L1 antibody moiety including a heavy chain and a light chain variable region sequence, where:

(a) the heavy chain sequence has at least 85% sequence identity to the heavy chain sequence:

(SEQ ID NO: 14)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA,

(b) the light chain sequence has at least 85% sequence identity to the light chain sequence:

(SEQ ID NO: 13)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In various embodiments, the heavy chain sequence has at least 86% sequence identity to SEQ ID NO: 14 and the light chain sequence has at least 86% sequence identity to SEQ ID NO: 13; the heavy chain sequence has at least 87% sequence identity to SEQ ID NO: 14 and the light chain sequence has at least 87% sequence identity to SEQ ID NO: 13; the heavy chain sequence has at least 88% sequence identity to SEQ ID NO: 14 and the light chain sequence has at least 88% sequence identity to SEQ ID NO: 13; the heavy chain sequence has at least 89% sequence identity to SEQ ID NO: 14 and the light chain sequence has at least 89% sequence identity to SEQ ID NO: 13; the heavy chain sequence has at least, 90% sequence identity to SEQ ID NO: 14 and the light chain sequence has at least 90% sequence identity to SEQ ID NO: 13; the heavy chain sequence has at least 91% sequence identity to SEQ ID NO: 14 and the light chain sequence has at least 91% sequence identity to SEQ ID NO: 13; the heavy chain sequence has at least 92% sequence identity to SEQ ID NO: 14 and the light chain sequence has at least 92% sequence identity to SEQ ID NO: 13; the heavy chain sequence has at least 93% sequence identity to SEQ ID NO: 14 and the light chain sequence has at least 93% sequence identity to SEQ ID NO: 13; the heavy chain sequence has at least 94% sequence identity to SEQ ID NO: 14 and the light chain sequence has at least 94% sequence identity to SEQ ID NO: 13; the heavy chain sequence has at least 95% sequence identity to SEQ ID NO: 14 and the light chain sequence has at least 95% sequence identity to SEQ ID NO: 13; the heavy chain sequence has at least 96% sequence identity to SEQ ID NO: 14 and the light chain sequence has at least 96% sequence identity to SEQ ID NO: 13; the heavy chain sequence has at least 97% sequence identity to SEQ ID NO: 14 and the light chain sequence has at least 97% sequence identity to SEQ ID NO: 13; the heavy chain sequence has at least 98% sequence identity to SEQ ID NO: 14 and the light chain sequence has at least 98% sequence identity to SEQ ID NO: 13; the heavy chain sequence has at least 99% sequence identity to SEQ ID NO: 14 and the light chain sequence has at least 99% sequence identity to SEQ ID NO: 13; or the heavy chain sequence comprises SEQ ID NO: 14 and the light chain sequence comprises SEQ ID NO: 13.

Further exemplary anti-PD-L1 antibodies that can be used in an anti-PD-L1/TGFβ Trap are described in US patent application publication US 2018/0334504.

In certain embodiments, the disclosure features an anti-PD-L1 antibody moiety including a heavy chain and a light chain variable region sequence, where

(a) the heavy chain sequence has at least 85% sequence identity to the heavy chain sequence:

and

(SEQ ID NO: 55)
QVQLQESGPGLVKPSQTLSLTCTVSGGSISNDYWTWIRQHPGKGLEYIGY
ISYTGSTYYNPSLKSRVTISRDTSKNQFSLKLSSVTAADTAVYYCARSGG
WLAPFDYWGRGTLVTVSS,

(b) the light chain sequence has at least 85% sequence identity to the light chain sequence:

(SEQ ID NO: 56)
DIVMTQSPDSLAVSLGERATINCKSSQSLFYHSNQKHSLAWYQQKPGQPP
KLLIYGASTRESGVPDRFSGSGSGTDFTLTISSLQAEDVAVYYCQQYYGY
PYTFGGGTKVEIK.

In various embodiments, the heavy chain sequence has at least 86% sequence identity to SEQ ID NO: 55 and the light chain sequence has at least 86% sequence identity to SEQ ID NO: 56; the heavy chain sequence has at least 87% sequence identity to SEQ ID NO: 55 and the light chain sequence has at least 87% sequence identity to SEQ ID NO: 56; the heavy chain sequence has at least 88% sequence identity to SEQ ID NO: 55 and the light chain sequence has at least 88% sequence identity to SEQ ID NO: 56; the heavy chain sequence has at least 89% sequence identity to SEQ ID NO: 55 and the light chain sequence has at least 89% sequence identity to SEQ ID NO: 56; the heavy chain sequence has at least, 90% sequence identity to SEQ ID NO: 55 and the light chain sequence has at least 90% sequence identity to SEQ ID NO: 56; the heavy chain sequence has at least 91% sequence identity to SEQ ID NO: 55 and the light chain sequence has at least 91% sequence identity to SEQ ID NO: 56; the heavy chain sequence has at least 92% sequence identity to SEQ ID NO: 55 and the light chain sequence has at least 92% sequence identity to SEQ ID NO: 56; the heavy chain sequence has at least 93% sequence identity to SEQ ID NO: 55 and the light chain sequence has at least 93% sequence identity to SEQ ID NO: 56; the heavy chain sequence has at least 94% sequence identity to SEQ ID NO: 55 and the light chain sequence has at least 94% sequence identity to SEQ ID NO: 56; the heavy chain sequence has at least 95% sequence identity to SEQ ID NO: 55 and the light chain sequence has at least 95% sequence identity to SEQ ID NO: 56; the heavy chain sequence has at least 96% sequence identity to SEQ ID NO: 55 and the light chain sequence has at least 96% sequence identity to SEQ ID NO: 56; the heavy chain sequence has at least 97% sequence identity to SEQ ID NO: 55 and the light chain sequence has at least 97% sequence identity to SEQ ID NO: 56; the heavy chain sequence has at least 98% sequence identity to SEQ ID NO: 55 and the light chain sequence has at least 98% sequence identity to SEQ ID NO: 56; the heavy chain sequence has at least 99% sequence identity to SEQ ID NO: 55 and the light chain sequence has at least 99% sequence identity to SEQ ID NO: 56; or the heavy chain sequence comprises SEQ ID NO: 55 and the light chain sequence comprises SEQ TD NO: 56

In certain embodiments, the disclosure features an anti-PD-L1 antibody moiety including a heavy chain and a light chain variable region sequence, where

(a) the heavy chain sequence has at least 85% sequence identity to the heavy chain sequence:

(SEQ ID NO: 57)
QVQLVQSGAEVKKPGASVKVSCKASGYTFTSYWMHWVRQAPGQGLEWMGR
IGPNSGFTSYNEKFKNRVTMTRDTSTSTVYMELSSLRSEDTAVYYCARGG
SSYDYFDYWGQGTTVTVSS,

and

(b) the light chain sequence has at least 85% sequence identity to the light chain sequence:

(SEQ ID NO: 58)
DIVLTQSPASLAVSPGQRATITCRASESVSIHGTHLMHWYQQKPGQPPKL
LIYAASNLESGVPARFSGSGSGTDFTLTINPVEAEDTANYYCQQSFEDPL
TFGQGTKLEIK.

In various embodiments, the heavy chain sequence has at least 86% sequence identity to SEQ ID NO: 57 and the light chain sequence has at least 86% sequence identity to SEQ ID NO: 58; the heavy chain sequence has at least 87% sequence identity to SEQ ID NO: 57 and the light chain sequence has at least 87% sequence identity to SEQ ID NO: 58; the heavy chain sequence has at least 88% sequence identity to SEQ ID NO: 57 and the light chain sequence has at least 88% sequence identity to SEQ ID NO: 58; the heavy chain sequence has at least 89% sequence identity to SEQ ID NO: 57 and the light chain sequence has at least 89% sequence identity to SEQ ID NO: 58; the heavy chain sequence has at least, 90% sequence identity to SEQ ID NO: 57 and the light chain sequence has at least 90% sequence identity to SEQ ID NO: 58; the heavy chain sequence has at least 91% sequence identity to SEQ ID NO: 57 and the light chain sequence has at least 91% sequence identity to SEQ ID NO: 58; the heavy chain sequence has at least 92% sequence identity to SEQ ID NO: 57 and the light chain sequence has at least 92% sequence identity to SEQ ID NO: 58; the heavy chain sequence has at least 93% sequence identity to SEQ ID NO: 57 and the light chain sequence has at least 93% sequence identity to SEQ ID NO: 58; the heavy chain sequence has at least 94% sequence identity to SEQ ID NO: 57 and the light chain sequence has at least 94% sequence identity to SEQ ID NO: 58; the heavy chain sequence has at least 95% sequence identity to SEQ ID NO: 57 and the light chain sequence has at least 95% sequence identity to SEQ TD NO: 58; the heavy chain sequence has at least 96% sequence identity to SEQ ID NO: 57 and the light chain sequence has at least 96% sequence identity to SEQ ID NO: 58; the heavy chain sequence has at least 97% sequence identity to SEQ TD NO: 57 and the light chain sequence has at least 97% sequence identity to SEQ ID NO: 58; the heavy chain sequence has at least 98% sequence identity to SEQ ID NO: 57 and the light chain sequence has at least 98% sequence identity to SEQ ID NO: 58; the heavy chain sequence has at least 99% sequence identity to SEQ ID NO: 57 and the light chain sequence has at least 99% sequence identity to SEQ ID NO: 58; or the heavy chain sequence comprises SEQ ID NO: 57 and the light chain sequence comprises SEQ ID NO: 58.

In certain embodiments, the disclosure features an anti-PD-L1 antibody moiety including a heavy chain and a light chain sequence, where

(a) the heavy chain sequence has at least 85% sequence identity to the heavy chain sequence:

(SEQ ID NO: 59)
QVQLQESGPGLVKPSQTLSLTCTVSGGSISNDYWTWIRQHPGKGLEYIGY
ISYTGSTYYNPSLKSRVTISRDTSKNQFSLKLSSVTAADTAVYYCARSGG
WLAPFDYWGRGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEAAGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK,

and

(b) the light chain sequence has at least 85% sequence identity to the light chain sequence:

(SEQ ID NO: 60)
DIVMTQSPDSLAVSLGERATINCKSSQSLFYHSNQKHSLAWYQQKPGQPP
KLLIYGASTRESGVPDRFSGSGSGTDFTLTISSLQAEDVAVYYCQQYYGY
PYTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSENRGEC.

In various embodiments, the heavy chain sequence has at least 86% sequence identity to SEQ ID NO: 59 and the light chain sequence has at least 86% sequence identity to SEQ ID NO: 60; the heavy chain sequence has at least 87% sequence identity to SEQ ID NO: 59 and the light chain sequence has at least 87% sequence identity to SEQ ID NO: 60; the heavy chain sequence has at least 88% sequence identity to SEQ ID NO: 59 and the light chain sequence has at least 88% sequence identity to SEQ ID NO: 60; the heavy chain sequence has at least 89% sequence identity to SEQ ID NO: 59 and the light chain sequence has at least 89% sequence identity to SEQ ID NO: 60; the heavy chain sequence has at least, 90% sequence identity to SEQ ID NO: 59 and the light chain sequence has at least 90% sequence identity to SEQ ID NO: 60; the heavy chain sequence has at least 91% sequence identity to SEQ ID NO: 59 and the light chain sequence has at least 91% sequence identity to SEQ ID NO: 60; the heavy chain sequence has at least 92% sequence identity to SEQ ID NO: 59 and the light chain sequence has at least 92% sequence identity to SEQ ID NO: 60; the heavy chain sequence has at least 93% sequence identity to SEQ ID NO: 59 and the light chain sequence has at least 93% sequence identity to SEQ ID NO: 60; the heavy chain sequence has at least 94% sequence identity to SEQ ID NO: 59 and the light chain sequence has at least 94% sequence identity to SEQ ID NO: 60; the heavy chain sequence has at least 95% sequence identity to SEQ ID NO: 59 and the light chain sequence has at least 95% sequence identity to SEQ ID NO: 60; the heavy chain sequence has at least 96% sequence identity to SEQ ID NO: 59 and the light chain sequence has at least 96% sequence identity to SEQ ID NO: 60; the heavy chain sequence has at least 97% sequence identity to SEQ ID NO: 59 and the light chain sequence has at least 97% sequence identity to SEQ ID NO: 60; the heavy chain sequence has at least 98% sequence identity to SEQ ID NO: 59 and the light chain sequence has at least 98% sequence identity to SEQ ID NO: 60; the heavy chain sequence has at least 99% sequence identity to SEQ ID NO: 59 and the light chain sequence has at least 99% sequence identity to SEQ ID NO: 60; or the heavy chain sequence comprises SEQ ID NO: 59 and the light chain sequence comprises SEQ ID NO: 60.

In certain embodiments, the disclosure features an anti-PD-L1 antibody moiety including a heavy chain and a light chain sequence, where

(a) the heavy chain sequence has at least 85% sequence identity to the heavy chain sequence:

(SEQ ID NO: 61)
QVQLVQSGAEVKKPGASVKVSCKASGYTFTSYWMHWVRQAPGQGLEWMGR
IGPNSGFTSYNEKFKNRVTMTRDTSTSTVYMELSSLRSEDTAVYYCARGG
SSYDYFDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEAAGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGA,

and

(b) the light chain sequence has at least 85% sequence identity to the light chain sequence:

(SEQ ID NO: 62)
DIVLTQSPASLAVSPGQRATITCRASESVSIHGTHLMHWYQQKPGQPPKL
LIYAASNLESGVPARFSGSGSGTDFTLTINPVEAEDTANYYCQQSFEDPL
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In various embodiments, the heavy chain sequence has at least 86% sequence identity to SEQ ID NO: 61 and the light chain sequence has at least 86% sequence identity to SEQ ID NO: 62; the heavy chain sequence has at least 87% sequence identity to SEQ ID NO: 61 and the light chain sequence has at least 87% sequence identity to SEQ ID NO: 62; the heavy chain sequence has at least 88% sequence identity to SEQ ID NO: 61 and the light chain sequence has at least 88% sequence identity to SEQ ID NO: 62; the heavy chain sequence has at least 89% sequence identity to SEQ ID NO: 61 and the light chain sequence has at least 89% sequence identity to SEQ ID NO: 62; the heavy chain sequence has at least, 90% sequence identity to SEQ ID NO: 61 and the light chain sequence has at least 90% sequence identity to SEQ ID NO: 62; the heavy chain sequence has at least 91% sequence identity to SEQ ID NO: 61 and the light chain sequence has at least 91% sequence identity to SEQ ID NO: 62; the heavy chain sequence has at least 92% sequence identity to SEQ ID NO: 61 and the light chain sequence has at least 92% sequence identity to SEQ ID NO: 62; the heavy chain sequence has at least 93% sequence identity to SEQ ID NO: 61 and the light chain sequence has at least 93% sequence identity to SEQ ID NO: 62; the heavy chain sequence has at least 94% sequence identity to SEQ ID NO: 61 and the light chain sequence has at least 94% sequence identity to SEQ ID NO: 62; the heavy chain sequence has at least 95% sequence identity to SEQ ID NO: 61 and the light chain sequence has at least 95% sequence identity to SEQ TD NO: 62; the heavy chain sequence has at least 96% sequence identity to SEQ ID NO: 61 and the light chain sequence has at least 96% sequence identity to SEQ ID NO: 62; the heavy chain sequence has at least 97% sequence identity to SEQ TD NO: 61 and the light chain sequence has at least 97% sequence identity to SEQ ID NO: 62; the heavy chain sequence has at least 98% sequence identity to SEQ ID NO: 61 and the light chain sequence has at least 98% sequence identity to SEQ ID NO: 62; the heavy chain sequence has at least 99% sequence identity to SEQ ID NO: 61 and the light chain sequence has at least 99% sequence identity to SEQ ID NO: 62; or the heavy chain sequence comprises SEQ ID NO: 61 and the light chain sequence comprises SEQ ID NO: 62.

Yet further exemplary anti-PD-L1 antibodies that can be used in an anti-PD-L1/TGFβ Trap are described in US patent publication U.S. Pat. No. 7,943,743.

In one embodiment of the disclosure, the anti-PD-L1 antibody is MDX-1105.

In certain embodiments, the anti-PD-L1 antibody is MEDI-4736.

### Constant Region

The proteins and peptides of the disclosure can include a constant region of an immunoglobulin or a fragment, analog, variant, mutant, or derivative of the constant region. In certain embodiments, the constant region is derived from a human immunoglobulin heavy chain, for example, IgG1, IgG2, IgG3, IgG4, or other classes. In certain embodiments, the constant region includes a CH2 domain. In certain embodiments, the constant region includes CH2 and CH3 domains or includes hinge-CH2-CH3. Alternatively, the constant region can include all or a portion of the hinge region, the CH2 domain and/or the CH3 domain.

In one embodiment, the constant region contains a mutation that reduces affinity for an Fc receptor or reduces Fc effector function. For example, the constant region can contain a mutation that eliminates the glycosylation site within the constant region of an IgG heavy chain. In some embodiments, the constant region contains mutations, deletions, or insertions at an amino acid position corresponding to Leu234, Leu235, Gly236, Gly237, Asn297, or Pro331 of IgG1 (amino acids are numbered according to EU nomenclature). In a particular embodiment, the constant region contains a mutation at an amino acid position corresponding to Asn297 of IgG1. In alternative embodiments, the constant region contains mutations, deletions, or insertions at an amino acid position corresponding to Leu281, Leu282, Gly283, Gly284, Asn344, or Pro378 of IgG1.

In some embodiments, the constant region contains a CH2 domain derived from a human IgG2 or IgG4 heavy chain. Preferably, the CH2 domain contains a mutation that eliminates the glycosylation site within the CH2 domain. In one embodiment, the mutation alters the asparagine within the Gln-Phe-Asn-Ser (SEQ ID NO: 15) amino acid sequence within the CH2 domain of the IgG2 or IgG4 heavy chain. Preferably, the mutation changes the asparagine to a glutamine. Alternatively, the mutation alters both the phenylalanine and the asparagine within the Gln-Phe-Asn-Ser (SEQ ID NO: 15) amino acid sequence. In one embodiment, the Gln-Phe-Asn-Ser (SEQ ID NO: 15) amino acid sequence is replaced with a Gln-Ala-Gln-Ser (SEQ ID NO: 16) amino acid sequence. The asparagine within the Gln-Phe-Asn-Ser (SEQ ID NO: 15) amino acid sequence corresponds to Asn297 of IgG1.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. More preferably the hinge region is derived from a human IgG1 heavy chain. In one embodiment the cysteine in the Pro-Lys-Ser-Cys-Asp-Lys (SEQ ID NO: 17) amino acid sequence of the IgG1 hinge region is altered. In certain embodiments, the Pro-Lys-Ser-Cys-Asp-Lys (SEQ ID NO: 17) amino acid sequence is replaced with a Pro-Lys-Ser-Ser-Asp-Lys (SEQ ID NO: 18) amino acid sequence. In certain embodiments, the constant region includes a CH2 domain derived from a first antibody isotype and a hinge region derived from a second antibody isotype. In certain embodiments, the CH2 domain is derived from a human IgG2 or IgG4 heavy chain, while the hinge region is derived from an altered human IgG1 heavy chain.

The alteration of amino acids near the junction of the Fc portion and the non-Fc portion can dramatically increase the serum half-life of the Fc fusion protein (PCT publication WO 0158957, the disclosure of which is hereby incorporated by reference). Accordingly, the junction region of a protein or polypeptide of the present disclosure can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life. In another embodiment, the constant region is derived from an IgG sequence in which the Leu-Ser-Leu-Ser (SEQ ID NO: 19) amino acid sequence near the C-terminus of the constant region is altered to eliminate potential junctional T-cell epitopes. For example, in one embodiment, the Leu-Ser-Leu-Ser (SEQ ID NO: 19) amino acid sequence is replaced with an Ala-Thr-Ala-Thr (SEQ ID NO: 20) amino acid sequence. In other embodiments, the amino acids within the Leu-Ser-Leu-Ser (SEQ ID NO: 19) segment are replaced with other amino acids such as glycine or proline. Detailed methods of generating amino acid substitutions of the Leu-Ser-Leu-Ser (SEQ ID NO: 19) segment near the C-terminus of an IgG1, IgG2, IgG3, IgG4, or other immunoglobulin class molecule have been described in U.S. Patent Publication No. 20030166877, the disclosure of which is hereby incorporated by reference.

Suitable hinge regions for the present disclosure can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a hinge region of the present disclosure is derived from IgG1, e.g., human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present disclosure can be derived from the IgG4 hinge region, preferably containing a mutation that enhances correct formation of disulfide bonds between heavy chain-derived moieties (Angal S., et al. (1993) Mol. Immunol., 30:105-8).

In accordance with the present disclosure, the constant region can contain CH2 and/or CH3 domains and a hinge region that are derived from different antibody isotypes, e.g., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 20030044423, the disclosure of which is hereby incorporated by reference.

In accordance with the present disclosure, the constant region can contain one or more mutations described herein. The combinations of mutations in the Fc portion can have additive or synergistic effects on the prolonged serum half-life and increased in vivo potency of the bifunctional molecule. Thus, in one exemplary embodiment, the constant region can contain (i) a region derived from an IgG sequence in which the Leu-Ser-Leu-Ser (SEQ ID NO: 19) amino acid sequence is replaced with an Ala-Thr-Ala-Thr (SEQ ID NO: 20) amino acid sequence; (ii) a C-terminal alanine residue instead of lysine; (iii) a CH2 domain and a hinge region that are derived from different antibody isotypes, for example, an IgG2 CH2 domain and an altered IgG1 hinge region; and (iv) a mutation that eliminates the glycosylation site within the IgG2-derived CH2 domain, for example, a Gln-Ala-Gln-Ser (SEQ ID NO: 16) amino acid sequence instead of the Gln-Phe-Asn-Ser (SEQ ID NO: 15) amino acid sequence within the IgG2-derived CH2 domain.

### Antibody Fragments

The proteins and polypeptides of the disclosure can also include antigen-binding fragments of antibodies. Exemplary antibody fragments include scFv, Fv, Fab, F(ab′)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Fragments of antibodies that have the same or comparable binding characteristics to those of the whole antibody may also be present. Such fragments may contain one or both Fab fragments or the F(ab′)₂ fragment. The antibody fragments may contain all six CDRs of the whole antibody, although fragments containing fewer than all of such regions, such as three, four or five CDRs, are also functional.

### Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th cd., 1985. For a brief review of methods for drug delivery, see, e.g., Langer (Science 249:1527-1533, 1990).

In one aspect, the present disclosure provides an intravenous drug delivery formulation for use in a method of treating TNBC that includes 500 mg-2400 mg of a protein including a first polypeptide and a second polypeptide, the first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), the second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, a protein product of the present disclosure includes a first polypeptide that includes the amino acid sequence of SEQ ID NO: 3, and a second polypeptide that includes the amino acid sequence of SEQ TD NO: 1. In certain embodiments, a protein product of the present disclosure includes a first polypeptide that comprises the amino acid sequences of SEQ ID NOs: 35, 36, and 37, and a second polypeptide that comprises the amino acid sequences of SEQ ID NOs: 38, 39, and 40.

In certain embodiments of the present disclosure, the intravenous drug delivery formulation for use in a method of treating TNBC may include an about 1200 mg to about 2400 mg dose (e.g., about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to 2400 mg, about 1400 mg to 2400 mg, about 1500 mg to 2400 mg, about 1600 mg to 2400 mg, about 1700 mg to 2400 mg, about 1800 mg to 2400 mg, about 1900 mg to 2400 mg, about 2000 mg to 2400 mg, about 2100 mg to 2400 mg, about 2200 mg to 2400 mg, or about 2300 mg to 2400 mg) of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 2100 to about 2000 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 2100 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 2100 mg dose of aprotein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 1200 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 1200 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 1800 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 1800 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)).

In certain embodiments, the intravenous drug delivery formulation may include an about 2400 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include an about 2400 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ TD NO: 1)). In certain embodiments, the intravenous drug delivery formulation may include a 2400 mg dose of a protein of the present disclosure (e.g., anti-PD-L1/TGFβ Trap (e.g., including a first polypeptide comprising the amino acid sequences of SEQ ID NOs: 35, 36, and 37, and a second polypeptide comprising the amino acid sequences of SEQ TD NOs: 38, 39, and 40)).

In certain embodiments, the intravenous drug delivery formulation may include a 1800 mg dose of a protein of the present disclosure (e.g., anti-PD-L1/TGFβ Trap (e.g., including a first polypeptide comprising the amino acid sequences of SEQ ID NOs: 35, 36, and 37, and a second polypeptide comprising the amino acid sequences of SEQ ID NOs: 38, 39, and 40)). In certain embodiments, the intravenous drug delivery formulation may include a 2100 mg dose of a protein of the present disclosure (e.g., anti-PD-L1/TGFβ Trap (e.g., including a first polypeptide comprising the amino acid sequences of SEQ ID NOs: 35, 36, and 37, and a second polypeptide comprising the amino acid sequences of SEQ ID NOs: 38, 39, and 40)). In certain embodiments, the intravenous drug delivery formulation may include a 2400 mg dose of a protein of the present disclosure (e.g., anti-PD-L1/TGFβ Trap (e.g., including a first polypeptide comprising the amino acid sequences of SEQ ID NOs: 35, 36, and 37, and a second polypeptide comprising the amino acid sequences of SEQ ID NOs: 38, 39, and 40)).

In certain embodiments, the intravenous drug delivery formulation for use in a method of treating TNBC may include an about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of a protein product of the present disclosure (e.g., anti-PD-L1/TGFβ Trap). In certain embodiments, the intravenous drug delivery formulation for use in a method of treating TNBC may include an about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of a protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40.

In certain embodiments, the intravenous drug delivery formulation for use in a method of treating TNBC may include about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, or about 2400 mg of the protein of the present disclosure (e.g., anti-PD-L1/TGFβ Trap comprising a first polypeptide that comprises the amino acid sequences of SEQ ID NOs: 35, 36, and 37, and a second polypeptide that comprises the amino acid sequences of SEQ ID NOs: 38, 39, and 40).

The intravenous drug delivery formulation of the present disclosure for use in a method of treating TNBC may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and about 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtain a therapeutic dose of the protein in the intravenous drug formulation. In certain embodiments, the formulation may be a liquid formulation of a protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40, and stored as about 250 mg/vial to about 2000 mg/vial (e.g., about 250 mg/vial to about 2000 mg/vial, about 250 mg/vial to about 1900 mg/vial, about 250 mg/vial to about 1800 mg/vial, about 250 mg/vial to about 1700 mg/vial, about 250 mg/vial to about 1600 mg/vial, about 250 mg/vial to about 1500 mg/vial, about 250 mg/vial to about 1400 mg/vial, about 250 mg/vial to about 1300 mg/vial, about 250 mg/vial to about 1200 mg/vial, about 250 mg/vial to about 1100 mg/vial, about 250 mg/vial to about 1000 mg/vial, about 250 mg/vial to about 900 mg/vial, about 250 mg/vial to about 800 mg/vial, about 250 mg/vial to about 700 mg/vial, about 250 mg/vial to about 600 mg/vial, about 250 mg/vial to about 500 mg/vial, about 250 mg/vial to about 400 mg/vial, about 250 mg/vial to about 300 mg/vial, about 300 mg/vial to about 2000 mg/vial, about 400 mg/vial to about 2000 mg/vial, about 500 mg/vial to about 2000 mg/vial, about 600 mg/vial to about 2000 mg/vial, about 700 mg/vial to about 2000 mg/vial, about 800 mg/vial to about 2000 mg/vial, about 900 mg/vial to about 2000 mg/vial, about 1000 mg/vial to about 2000 mg/vial, about 1100 mg/vial to about 2000 mg/vial, about 1200 mg/vial to about 2000 mg/vial, about 1300 mg/vial to about 2000 mg/vial, about 1400 mg/vial to about 2000 mg/vial, about 1500 mg/vial to about 2000 mg/vial, about 1600 mg/vial to about 2000 mg/vial, about 1700 mg/vial to about 2000 mg/vial, about 1800 mg/vial to about 2000 mg/vial, or about 1900 mg/vial to about 2000 mg/vial). In certain embodiments, the formulation may be a liquid formulation and stored as about 600 mg/vial. In certain embodiments, the formulation may be a liquid formulation and stored as about 1200 mg/vial. In certain embodiments, the formulation may be a liquid formulation and stored as about 1800 mg/vial. In certain embodiments, the formulation may be a liquid formulation and stored as about 2400 mg/vial. In certain embodiments, the formulation may be a liquid formulation and stored as about 250 mg/vial.

This disclosure provides a liquid aqueous pharmaceutical formulation including a therapeutically effective amount of the protein of the present disclosure (e.g., anti-PD-L1/TGFβ Trap) in a buffered solution forming a formulation for use in a method of treating TNBC.

These compositions for use in a method of treating TNBC may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides for use in a method of treating TNBC, a formulation with an extended shelf life including a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)), in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation for use in a method of treating TNBC is prepared including a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40) in a pH-buffered solution. The buffer of this invention may have a pH ranging from about 4 to about 8, e.g., from about 4 to about 8, from about 4.5 to about 8, from about 5 to about 8, from about 5.5 to about 8, from about 6 to about 8, from about 6.5 to about 8, from about 7 to about 8, from about 7.5 to about 8, from about 4 to about 7.5, from about 4.5 to about 7.5, from about 5 to about 7.5, from about 5.5 to about 7.5, from about 6 to about 7.5, from about 6.5 to about 7.5, from about 4 to about 7, from about 4.5 to about 7, from about 5 to about 7, from about 5.5 to about 7, from about 6 to about 7, from about 4 to about 6.5, from about 4.5 to about 6.5, from about 5 to about 6.5, from about 5.5 to about 6.5, from about 4 to about 6.0, from about 4.5 to about 6.0, from about 5 to about 6, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation for use in a method of treating TNBC includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes about 1-1.5 mg/ml of citric acid, about 0.25 to about 0.5 mg/ml of sodium citrate, about 1.25 to about 1.75 mg/ml of disodium phosphate dihydrate, about 0.7 to about 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also alter with respect to the molecular weight of the polyol. For example, a lower amount of a monosaccharide (e.g. mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to about 15 mg/ml. In certain embodiments, the concentration of mannitol may be about 10-about 14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hilfsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

### Lyophilized Formulation

The lyophilized formulation for use in a method of treating TNBC of the present disclosure includes the anti-PD-L1/TGFβ Trap molecule and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between about 6 to about 8. In certain embodiments, the pH range for the lyophilized drug product may be from about 7 to about 8.

In certain embodiments, a salt or buffer components may be added in an amount of about 10 mM-about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product for use in a method of treating TNBC or inhibiting tumor growth in a cancer patient may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

### Liquid Formulation

In embodiments, the protein product of the present disclosure is formulated as a liquid formulation for use in a method of treating TNBC. The liquid formulation may be presented at a 10 mg/mL concentration in either a USP/Ph Eur type I 5 OR vial closed with a rubber stopper and sealed with an aluminum crimp seal closure. The stopper may be made of elastomer complying with USP and Ph Eur. In certain embodiments vials may be filled with about 61.2 mL of the protein product solution in order to allow an extractable volume of 60 mL. In certain embodiments, the liquid formulation may be diluted with 0.9% saline solution. In certain embodiments vials may contain about 61.2 mL of the protein product (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)) solution of about 20 mg/mL to about 50 mg/mL (e.g., about 20 mg/mL, about 25 mg/mL, about 30 mg/mL, about 35 mg/mL, about 40 mg/mL, about 45 mg/mL or about 50 mg/mL) in order to allow an extractable volume of 60 mL for delivering about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of the protein product (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40)) to a subject.

In certain embodiments, vials may contain about 61.2 mL of the protein product solution (protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40) of about 20 mg/mL to about 50 mg/mL (e.g., about 20 mg/mL, about 25 mg/mL, about 30 mg/mL, about 35 mg/mL, about 40 mg/mL, about 45 mg/mL or about 50 mg/mL) in order to allow an extractable volume of 60 mL for delivering about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of the protein product to a subject.

In certain embodiments, the liquid formulation for use in a method of treating TNBC or inhibiting tumor growth in a cancer patient of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 unit mass decrease of the parent peptide. The subsequent hydrolysis results in an 18 unit mass increase. Isolation of the succinimide intermediate is difficult due to instability under aqueous conditions. As such, deamidation is typically detectable as 1 unit mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation for use in a method of treating TNBC or inhibiting tumor growth in a cancer patient of the present disclosure may be preserved under conditions of pH and humidity to prevent deamidation of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (TV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is usefiil for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The description above describes multiple aspects and embodiments of the invention. The patent application specifically contemplates all combinations and permutations of the aspects and embodiments.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Identification of HMGA2 and MECOM as Predictors Among TNBC Patients for Response to an Anti-PD-L1/TGFβ Trap Protein Therapy

This example relates to a method that identified HMGA2 and MECOM as two predictors of responsiveness to an anti-PD-L1/TGFβ protein therapy among TNBC patients. 33 TNBC patients were treated with anti-PD-L1/TGFβ Trap bifunctional protein, and tumor samples from these patients were analyzed to distinguish responders versus non-responders to treatment with anti-PD-L1/TGFβ Trap protein.

Each of 30 tumor samples was annotated by a board-certified pathologist. RNA was extracted from three whole-slide scrapes of 4-5 μm-thick sections with a tumor content >50%, using Recoverall Total Nucleic Acid Isolation Kit for formalin-fixed, paraffin-embedded samples (ThermoFisher Scientific). 200 ng of total RNA, quantified using RiboGreen® RNA reagent (Life Technologies), was depleted of ribosomal RNA using the Ribo-Zero Gold rRNA Removal Kit (Illumina). Strand-specific libraries were prepared using the NEBNext Ultra Directional RNA Library Prep Kit (NEB) and sequenced on an HiSeq2500 (Illumina) using 2×50 base-pair paired-end sequencing. Approximately 100 million reads per sample were obtained.

Patient response to anti-PD-L1/TGFβ Trap treatment was coded using RECIST 1.1 criteria. Gene expression levels among responders (a group of subjects with best overall response of stable disease, partial response, or complete response) were compared to levels among non-responders (subjects with best overall response of progressive disease).

Sequencing reads were aligned against the Ensembl 75 human genome (GRCh37 February 2014) using Bowtie2 version 2.2.3. (Langmead, Nat Methods, 9:357-359 (2012)). Gene expression was determined using RSEM version 1.2.31 with the Ensembl gene annotations. (Li, BMC Bioinformatics 12:323(2011)). One outlier sample with abnormally few detectable genes was excluded from further analysis. Hypothesis testing was performed by comparing RSEM-computed expected counts. Transcript-per-million (TPM) values were upper-quartile normalized and log-transformed for further analysis.

Descriptive statistical analyses were performed using R version 3.3.1. (Hornik K, The R FAQ, available at https://CRAN.R-project.org/doc/FAQ/R-FAQ.html). Correlation coefficients were calculated using the Pearson method. Significance of differential expression was established using the ‘R’ package ‘DESeq2’ for individual genes; FDR-corrected p values <0.05 were considered statistically significant. Plots were generated using the ‘R’ package ‘ggplot2’.

Of 60,234 annotated transcripts that were tested, a set of candidate biomarkers was identified by filtering according to the following requirements: (1) Require differential expression; (2) require protein coding genes; (3) require minimum expression; (4) require separation of groups.

Require differential expression: All annotated genes were first tested using DESeq2 as described above; q values were obtained, denoting the significance of differential expression. Any gene with q value greater than 0.05 was rejected.

Require protein coding genes: BiomaRt was used to establish whether the Ensembl gene identifier for each transcript we mapped corresponded to a protein coding gene model. The “gene_biotype” for each gene ID from biomaRt was obtained, and categorized as either coding or non-coding. The following biotypes were categorized as non-coding: 3prime_overlapping_ncrna, antisense, IG_C_pseudogene, IG_J_pseudogene, IG_V_pseudogene, lincRNA, miRNA, misc_RNA, Mt_rRNA, Mt_tRNA, pseudogene, rRNA, sense_intronic, sense_overlapping, snoRNA, snRNA, TR_J_pseudogene, and TR_V_pseudogene. The following biotypes were categorized as coding: TR_D_gene, TR_C_gene, IG_C_gene, IG_J_gene, IG_D_gene, polymorphic_pseudogene, TR_J_gene, TR_V_gene. IG_V_gene, processed_transcript, protein_coding. Genes with a non-coding biotype were rejected as biomarker candidates due to low interpretability.

Require minimum expression: For each gene, the median TPM among responders and among non-responders was identified. If the median TPM for a given gene was below 0.5 in responders and in non-responders, that gene was rejected as a biomarker candidate.

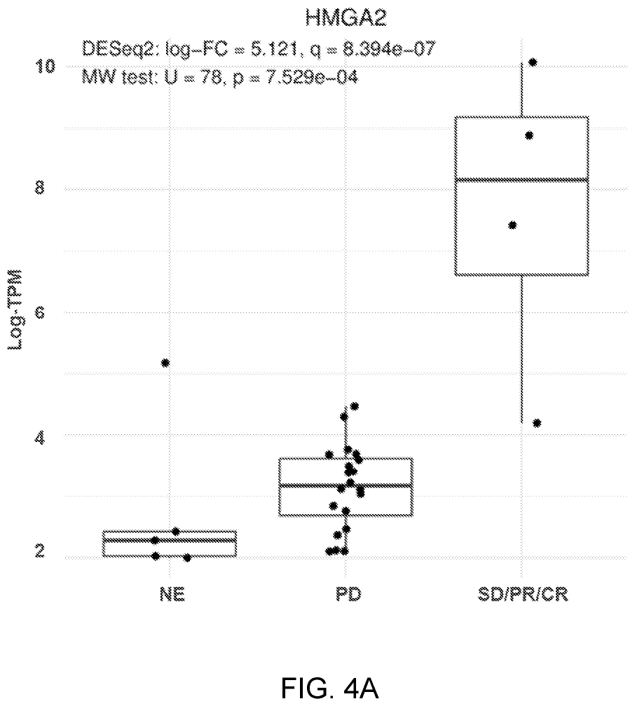

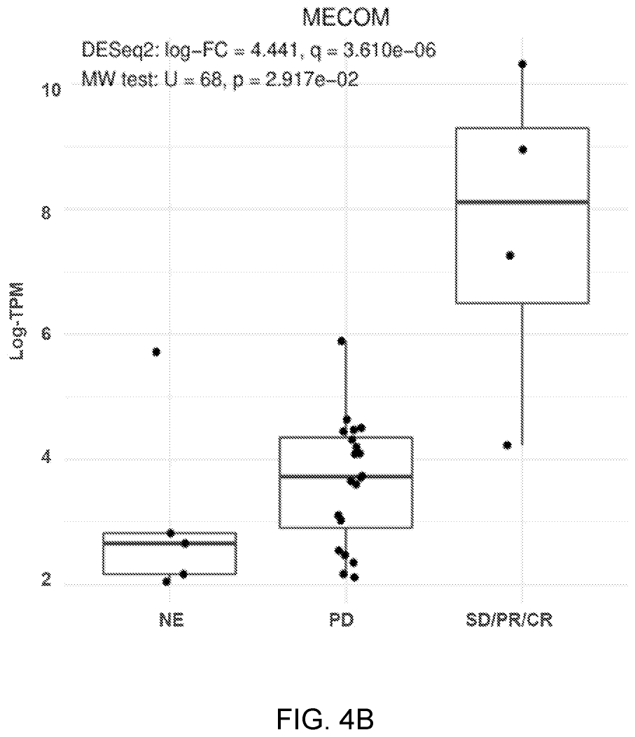

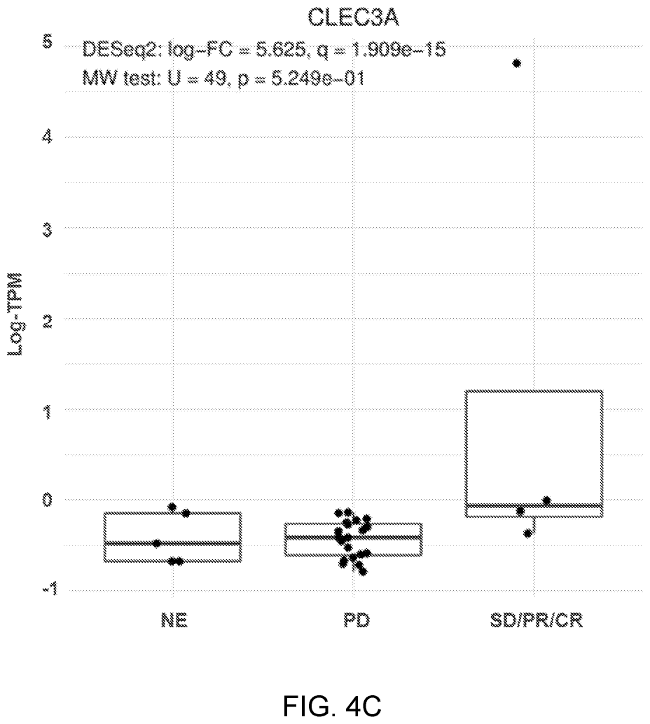

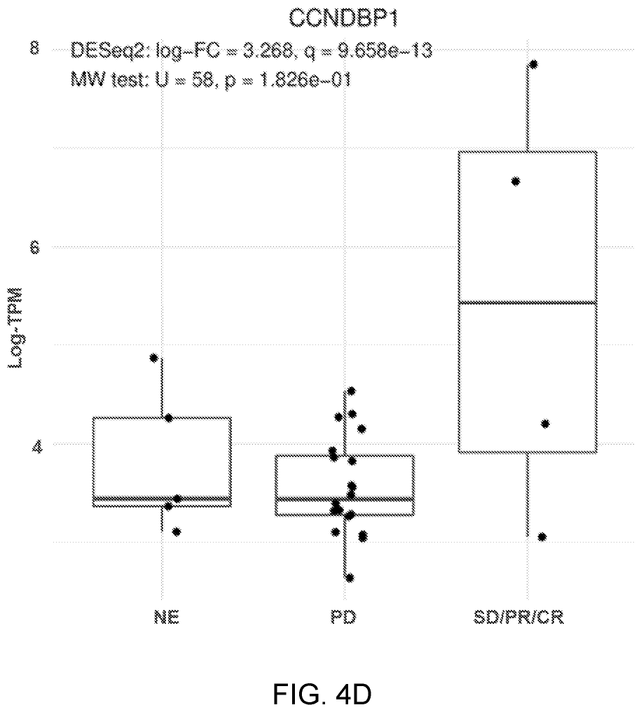

Require separation of groups: an ideal predictive biomarker separates responders from non-responders with precision. DESeq2 assesses the difference in mean expression between groups, but the difference of means may be called significant even if there is noticeable overlap in the levels of expression in each group (see FIGS. 4A-D). In addition, DESeq2 can be sensitive to large outliers. To correct for both issues, a Mann-Whitney U test was applied to test separation of groups. All genes whose nominal p-value exceeded 0.05 were rejected.

28 genes were identified as meeting all requirements. Of those 28 genes, two genes, HMGA2 and MECOM, were identified as having the greatest statistical significance of all analyzed genes. FIGS. 4A-D show box plots of log-TPM of several potential predictive biomarkers plotted against response status of patients treated with anti-PD-L1/TGFβ Trap protein. FIG. 4A shows a box plot of log-TPM of HMGA2 plotted against response status of patients treated with anti-PD-L1/TGFβ Trap protein. FIG. 4B shows a box plot of log-TPM of MECOM plotted against response status of patients treated with anti-PD-L1/TGFβ Trap protein. FIG. 4C shows a box plot of log-TPM of CLEC3A plotted against response status of patients treated with anti-PD-L1/TGFβ Trap protein. FIG. 4D shows a box plot of log-TPM of CCNDBP1 plotted against response status of patients treated with anti-PD-L1/TGFβ Trap protein.

To demonstrate the effect of the separation of groups requirement, expression levels for CLEC3A and CCNDBP1 were also analyzed. These two genes had the lowest DESeq2 computed q values among all genes passing the differential expression, protein coding, and minimum expression requirements, but they failed the separation of groups requirement (see FIGS. 4C-D (NE data was excluded from hypothesis testing)). Large fold-changes for these genes were observed in both responders from non-responders. The large fold-change computed for CLEC3A appeared to be driven by a single, large outlier, and gene expression among the remaining responders was only modestly elevated (see FIG. 4C (NE data was excluded from hypothesis testing)). Although CCNDBP1 expression was elevated 9.6-fold among responders, CCNDBP1 expression levels among responders spanned almost the entire range of expression among non-responders (see FIG. 4D (NE data was excluded from hypothesis testing)). The large p values obtained from the Mann-Whitney U test for these genes accurately reflect these shortcomings.

Increased expression of HMGA2 and MECOM genes were shown to be associated with poor prognosis in breast cancer (Wu et al., Cancer Letters 2016; Wang et al., Cancer Research 2017). This association suggested that the observation of a positive association with response was not confounded by reduced disease severity among responders. In addition, both genes have well-known association with TGFβ biology (Thualt et al., Cell Biology 2006; Liu et al., Oncogene 2006), supporting a mechanistic explanation for their predictive power in treating TNBC patients with the anti-PD-L1/TGFb trap of the present invention.

As shown in FIGS. 2A (HMGA2) and 3B (MECOM), both HMGA2 and MECOM were found to be over-expressed by more than 20-fold in responders as compared with non-responders. In FIG. 2A, HMGA2 expression levels (log of transcripts per million (TPM)) are plotted against response to anti-PD-L1/TGFβ Trap protein (PD=progressive disease; SD=stable disease; PR=partial response). High HMGA2 expression is considered as the expression level at least as high as the lowest HMGA2 expression among patients who responds to anti-PD-L1/TGFβ Trap protein treatment. As shown in FIG. 2A, HMGA2 expression was found to be significantly higher (at least 35-fold) compared to the expression levels among the non-responders. NE data was excluded from hypothesis testing.

In FIG. 3B, MECOM expression levels (log of transcripts per million (TPM)) are plotted against response to anti-PD-L1/TGFβ Trap protein (PD=progressive disease; SD=stable disease; PR=partial response). High MECOM expression is considered as the expression level at least as high as the lowest MECOM expression among patients who responds to anti-PD-L1/TGFβ Trap protein treatment. As shown in FIG. 3B, MECOM expression was found to be significantly higher (at least 20-fold) compared to the expression levels among the non-responders. NE data was excluded from hypothesis testing.

In order to test whether a clinical response can be predicted in TNBC patients treated with an anti-PD-L1 antibody, data were extracted from metastatic breast cancer subjects in metastatic breast cancer cohort (METBRC). This study was performed to distinguish responders versus non-responders to treatment with an anti-PD-L1 antibody. In this study, metastatic breast cancer patients were treated with an anti-PD-L1 antibody. Three different categories were considered: (1) human epidermal growth factor receptor 2 positive, “HER2+”, (2) human epidermal growth factor receptor 2 negative, (estrogen receptor positive or progesterone receptor positive), “HER2−, (ER+ or PR+)”, and (3) human epidermal growth factor receptor 2 negative, (estrogen receptor negative and progesterone receptor negative), “HER2−, (ER− and PR−)”. While the latter group corresponded to triple negative breast cancer (TNBC), the first 2 groups were considered non-TNBC.

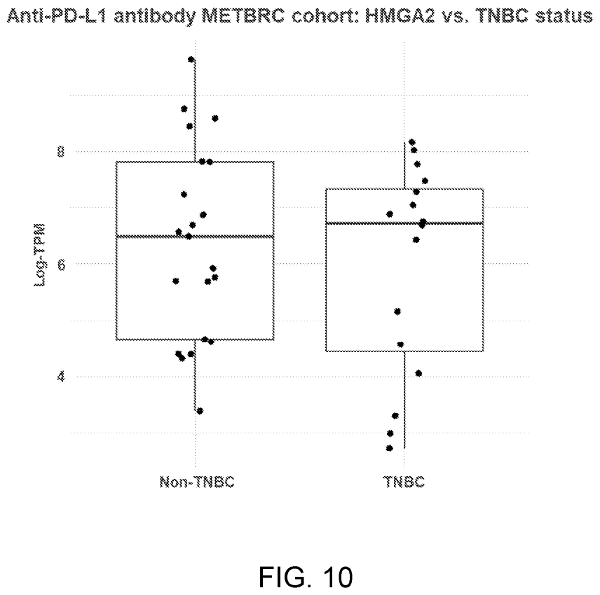

RNAseq data was available for 16 TNBC subjects and for 21 non-TNBC treated with an anti-PD-L1 antibody. FIG. 10 is a box plot showing expression of HMGA2 (in transcript per million, TPM) and TNBC status of subjects treated with an anti-PD-L1 antibody. FIG. 10 shows that there was no apparent difference in expression of the HMGA2 gene between the subjects with non-TNBC versus those with TNBC treated with an anti-PD-L1 antibody.

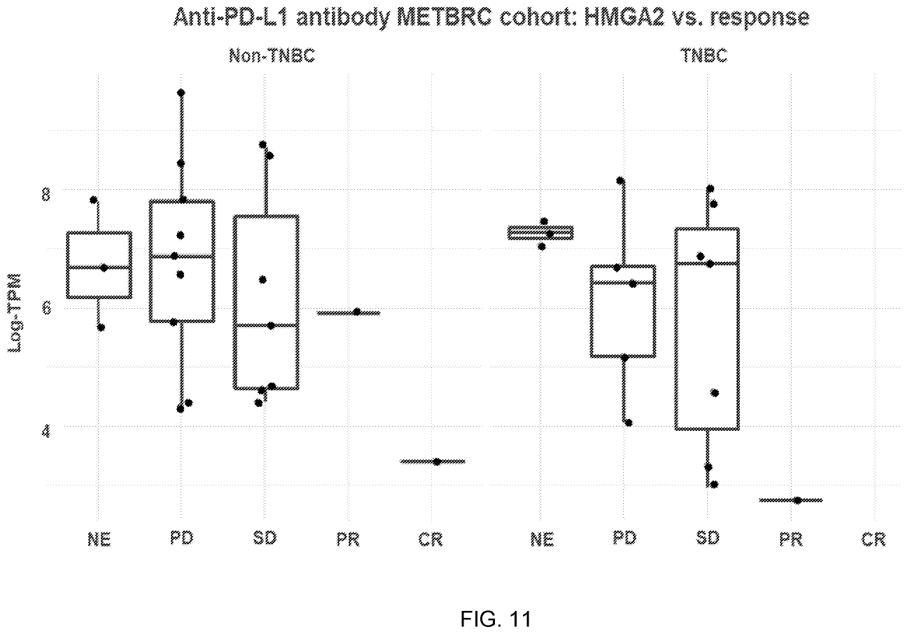

HMGA2 expression and its association with clinical response to an anti-PD-L1 antibody was also evaluated. FIG. 11 shows HMGA2 expression (in TPM) versus response in separate panels for non-TNBC (left) and for TNBC (right) subjects treated with an anti-PD-L1 antibody. As shown in FIG. 11, low expression levels of HMGA2 gene were present in 1 subject with non-TNBC with a complete response (CR), while all other subjects showed overlapping levels of HMGA2 expression regardless of response to anti-PD-L1 antibody. Low expression levels of HMGA2 gene were present in 1 subject with TNBC with a partial response (PR), while all other subjects showed overlapping levels of HMGA2 expression. In both cases (non-TNBC and TNBC), there appears to be no clear difference between responders (CR/PR) and non-responders (stable disease, SD/progressive disease, PD).

The data presented in this example shows that a clinical response can be predicted in TNBC patients treated with the anti-PD-L1/TGFβ trap of the present invention. The prediction of clinical response is not clear when the TNBC patients are treated with only anti-PD-L1 antibody.

### Example 2: Evaluation of Association Between HMGA2 Expression and TGF-β Signaling

In order to evaluate the association between HMGA2 expression and TGF-β signaling, animal studies were carried out. Briefly, orthotopic tumor injection was performed by injecting 0.2×10⁶ viable 4T1 cells suspended in 0.1 mL 1×PBS into the mammary fat pad of 8-10 week old Balb/C mice. Once tumor volume reached 100-150 mm³, mice were randomized and assigned to one of the following treatment groups: control, trap control, anti-PD-L1, and anti-PD-L1/TGFβ Trap. Mice in the control group were dosed with 400 μg of isotype control (hIgG1)—anti-PD-L1(mut); mice in the trap control were dosed with 492 μg of anti-PD-L1(mut)/TGFβ trap (anti-PD-L1(mut)/TGFβ Trap fusion protein contains an analogous heavy chain fusion polypeptide (SEQ ID No: 7) and a light chain with the mutations A31G, D52E, R99Y in the variable region that abrogate the binding to PD-L1 (SEQ ID No: 6)); mice in anti-PD-L1 group were dosed with 400 μg of anti-PD-L1; and mice in anti-PD-L1/TGFβ Trap group were dosed with 492 μg of anti-PD-L1/TGFβ Trap via intravenous injection once every three weeks (QDx3). Experimental animals were euthanized on day 6 and tumor samples were harvested.

RNAseq was performed on the tumor tissue samples harvested from the four treatment groups. Raw sequencing data was processed with standard quality control (QC) and alignment pipeline as described in Example 1 with the exception that sequencing reads were mapped against the Ensembl 75 mouse genome (GRCm38 February 2014). Normalized expression data was generated and used for the HMGA2 and TGF-β related gene expression analysis.

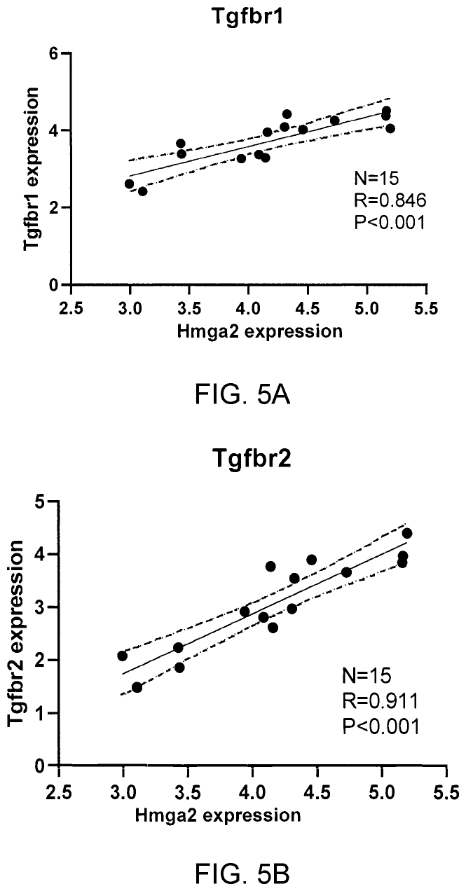

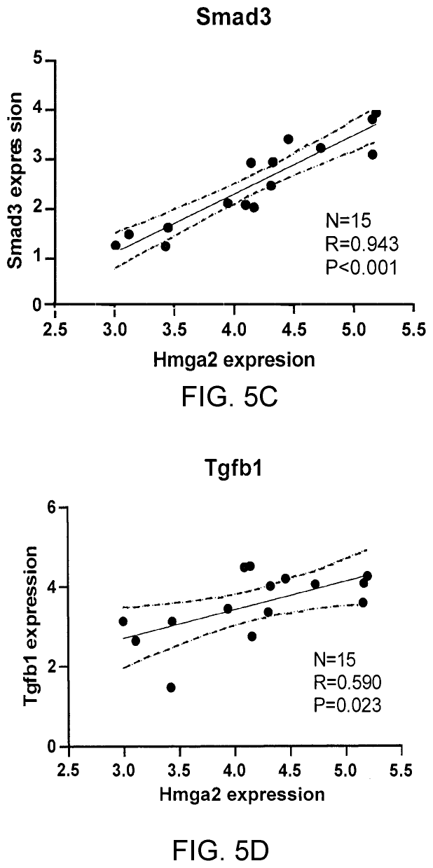

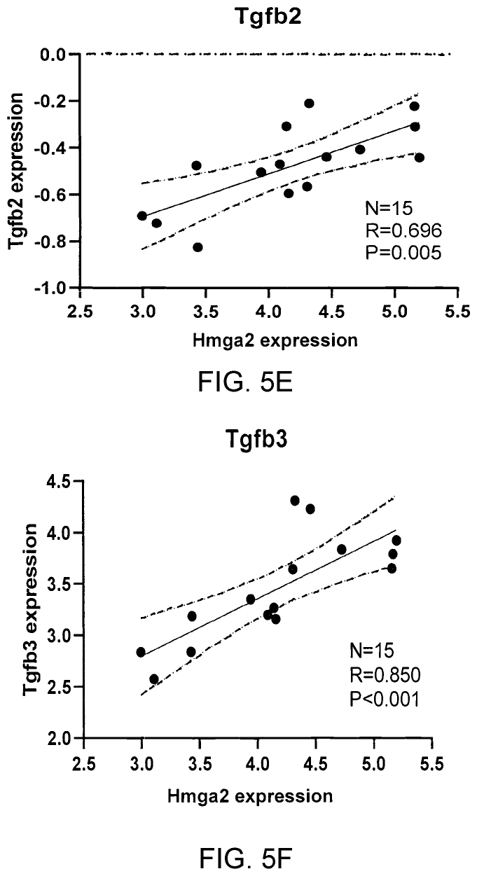

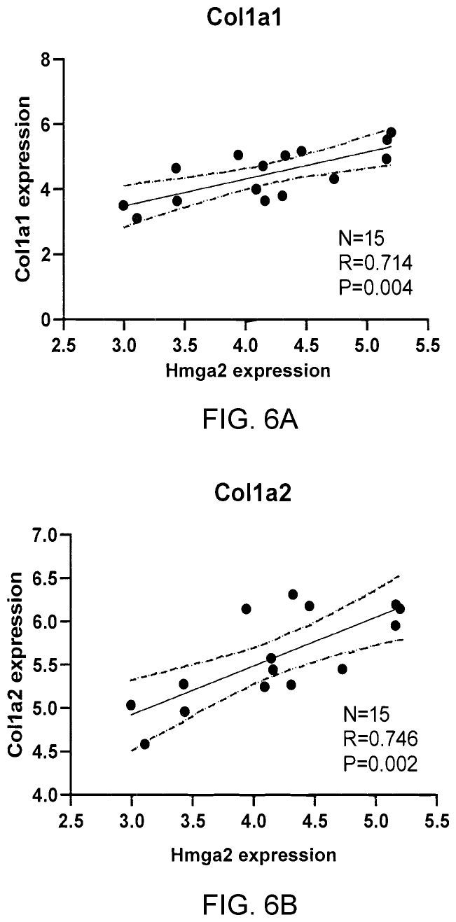

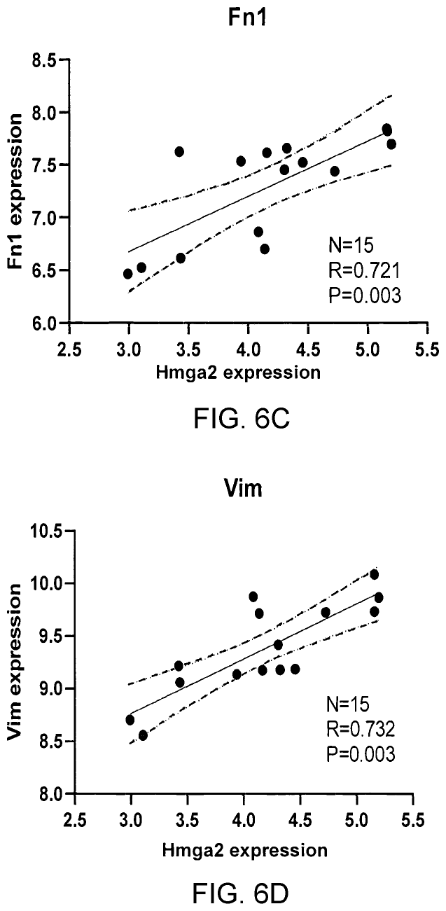

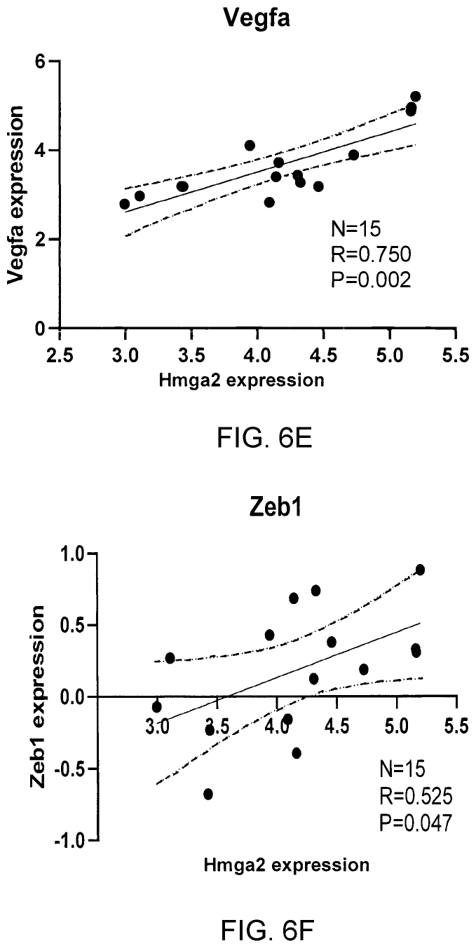

To evaluate the association between HMGA2 expression and TGF-β signaling, Spearman correlation analysis was performed on HMGA2 and a TGF-β gene signature (see Korkut et al., Cell Syst. 2018, 7, 422-437.e7). Separate analyses was done on the control animals (N=12) and anti-PD-L1/TGFβ Trap treated animals (N=15). In the control group, 30% (27/89) of the HMGA2/TGF-β signaling gene pairs showed a statistically significant R. In other words, expression of 27 TGF-β signaling genes correlated with expression of HMGA2 in RNA extracted from control-treated mice. Table 1 lists Spearman correlation R and p values associated with gene pairs (HMGA2 and TGF-β signaling genes) in control-treated animals. In the anti-PD-L1/TGFβ Trap treatment group, 55% (48/89) of the HMGA2/TGF-β signaling gene pairs showed a statistically significant R. Treatment with the anti-PD-L1/TGFβ Trap induced TGF-β specific transcriptomic changes and resulted in correlation of 48 TGF-β signaling genes expression with HMGA2 expression. Table 2 lists Spearman correlation R and p value associated with gene pairs in anti-PD-L1/TGFβ Trap treatment group. FIGS. 5A-F are scatter plots showing association between HMGA2 and selected TGF-β signaling core genes Tgfbr1, Tgfbr2, Smad3, Tgfb1, Tgfb2, and Tgfb3, respectively. FIG. 5A is a scatter plot showing association between HMGA2 expression and Tgfbr1 expression. FIG. 5B is a scatter plot showing association between HMGA2 and Tgfbr2 expression. FIG. 5C is a scatter plot showing association between HMGA2 expression and Smad3 expression. FIG. 5D is a scatter plot showing association between HMGA2 expression and Tgfb1 expression. FIG. 5E is a scatter plot showing association between HMGA2 expression and Tgfb2 expression. FIG. 5F is a scatter plot showing association between HMGA2 expression and Tgfb3 expression. FIGS. 6A-F are scatter plots showing association between HMGA2 and selected TGF-β signaling target genes Col1a1, Col1a2, Fn1, Vim, Vegfa, and Zeb1, respectively. FIGS. 6A-F are scatter plots showing association between HMGA2 and selected TGF-β signaling target genes. FIG. 6A is a scatter plot showing association between HMGA2 expression and Col1a1 expression. FIG. 6B is a scatter plot showing association between HMGA2 expression and Col1a2 expression. FIG. 6C is a scatter plot showing association between HMGA2 expression and Fn1 expression. FIG. 6D is a scatter plot showing association between HMGA2 expression and Vim expression. FIG. 6E is a scatter plot showing association between HMGA2 expression and Vegfa expression. FIG. 6F is a scatter plot showing association between HMGA2 expression and Zeb1 expression. These plots show that HMGA2 expression is associated with expression of TGF-β signaling pathway genes.

TABLE 1
Spearman correlation R and p value associated with HMGA2
and TGF-β signaling pathway genes in control animals.

| Gene pair | Spearman R | P-value |
|---|---|---|
| Hmga2-Acvr2b | 0.82517483 | 0.001592 |
| Hmga2-Zfyve9 | 0.8041958 | 0.002518 |
| Hmga2-Bmpr2 | 0.77622378 | 0.004331 |
| Hmga2-Snai1 | 0.77622378 | 0.004331 |
| Hmga2-Runx1 | 0.74825175 | 0.006974 |
| Hmga2-Fn1 | 0.73426573 | 0.008667 |
| Hmga2-Smad3 | 0.72027972 | 0.010637 |
| Hmga2-Bmp7 | 0.71328671 | 0.011766 |
| Hmga2-Pja2 | 0.6993007 | 0.014208 |
| Hmga2-Cdkn2b | −0.69230769 | 0.015546 |
| Hmga2-Inhbb | 0.68531469 | 0.017015 |
| Hmga2-Tgfb3 | 0.67832168 | 0.018522 |
| Hmga2-Stat3 | 0.67832168 | 0.018522 |
| Hmga2-Smadl | 0.67132867 | 0.020194 |
| Hmga2-Bcl2l11 | 0.66433566 | 0.021901 |
| Hmga2-Tgfb1 | 0.65034965 | 0.0257 |
| Hmga2-Smad6 | 0.65034965 | 0.0257 |
| Hmga2-Myc | 0.64335664 | 0.027801 |
| Hmga2-Inhbe | 0.63810013 | 0.030102 |
| Hniga2-Foxo3 | 0.62937063 | 0.032296 |
| Hmga2-Vim | 0.62937063 | 0.032296 |
| Hmga2-Acvr1 | 0.61538462 | 0.037277 |
| Hmga2-Acvr1b | 0.61538462 | 0.037277 |
| Hmga2-Runx2 | 0.61538462 | 0.037277 |
| Hmga2-Tert | 0.61538462 | 0.037277 |
| Hmga2-Gadd45b | −0.59440559 | 0.045705 |
| Hmga2-Pard6a | −0.57342657 | 0.055552 |
| Hmga2-Bmp4 | −0.56643357 | 0.059053 |
| Hmga2-Col1a1 | 0.56643357 | 0.059053 |
| Hmga2-Zeb2 | 0.55944056 | 0.062845 |
| Hmga2-Foxk1 | 0.53846154 | 0.074952 |
| Hmga2-Dab2 | 0.53146853 | 0.079434 |
| Hmga2-pja1 | 0.53146853 | 0.079434 |
| Hmga2-Sptbn1 | 0.51838959 | 0.086831 |
| Hniga2-Tgfbr3 | 0.48251748 | 0.11541 |
| Hmga2-Smad4 | 0.48251748 | 0.11541 |
| Hmga2-Dapk1 | 0.46853147 | 0.127507 |
| Hmga2-Foxo1 | 0.46853147 | 0.127507 |
| Hmga2-Foxo4 | 0.46853147 | 0.127507 |
| Hmga2-Inhba | 0.45454545 | 0.140439 |
| Hmga2-Tgfbrap1 | 0.45454545 | 0.140439 |
| Hmga2-Smad7 | 0.44055944 | 0.154234 |
| Hmga2-Smad7 | 0.44055944 | 0.154234 |
| Hmga2-Bmp10 | −0.48038446 | 0.166667 |
| Hmga2-Cdh1 | 0.42657343 | 0.168896 |
| Hmga2-Il6 | −0.41958042 | 0.176733 |
| Hmga2-Jun | 0.40559441 | 0.192767 |
| Hmga2-Col1a2 | 0.38461538 | 0.218343 |
| Hmga2-Cdknla | 0.37762238 | 0.227594 |
| Hmga2-Vegfa | 0.37062937 | 0.236687 |
| Hmga2-Bmp6 | −0.3677764 | 0.238531 |
| Hmga2-Gdf1 | −0.35916384 | 0.252159 |
| Hmga2-Snai2 | −0.33566434 | 0.286861 |
| Hmga2-Itgb6 | −0.32167832 | 0.308488 |
| Hmga2-Runx3 | 0.31468531 | 0.319405 |
| Hmga2-Gdf11 | 0.30769231 | 0.331041 |
| Hmga2-Smad2 | −0.3006993 | 0.342415 |
| Hmga2-Fos | 0.29370629 | 0.354539 |
| Hmga2-Bmpr1a | 0.26573427 | 0.404188 |
| Hmga2-Serpine1 | 0.24475524 | 0.443418 |
| Hmga2-Tgfbr2 | 0.23776224 | 0.457295 |
| Hmga2-Tgfb2 | 0.23076923 | 0.470802 |
| Hmga2-Bmpr1b | −0.23076923 | 0.470802 |
| Hmga2-Cdh2 | −0.22377622 | 0.485124 |
| Hmga2-Id1 | −0.21678322 | 0.499001 |
| Hmga2-Acvr2a | −0.2027972 | 0.527959 |
| Hmga2-Acvrl1 | 0.1958042 | 0.543064 |
| Hmga2-Tgfbr1 | 0.18881119 | 0.557684 |
| Hmga2-Twist1 | 0.18881119 | 0.557684 |
| Hmga2-Foxic2 | 0.18181818 | 0.573147 |
| Hmga2-Mmp2 | 0.18181818 | 0.573147 |
| Hmga2-Nodal | 0.15384615 | 0.635329 |
| Hmga2-Acvr1c | −0.14502276 | 0.654079 |
| Hmga2-Aldh1a1 | −0.12587413 | 0.699883 |
| Hmga2-Col3a1 | 0.11188811 | 0.732948 |
| Hmga2-Bmp15 | −0.09122863 | 0.778113 |
| Hmga2-Bmp2 | 0.09090909 | 0.782983 |
| Hmga2-Smad5 | −0.08391608 | 0.800385 |
| Hmga2-Mapk14 | 0.08391608 | 0.800385 |
| Hmga2-Bmp5 | 0.07746671 | 0.816129 |
| Hmga2-Inha | 0.06293706 | 0.851456 |
| Hmga2-Igf2 | 0.06293706 | 0.851456 |
| Hmga2-Twist2 | −0.06293706 | 0.851456 |
| Hmga2-Bmp3 | −0.04903685 | 0.881392 |
| Hmga2-Zeb1 | −0.03496503 | 0.920974 |
| Hmga2-Itgb8 | −0.01398601 | 0.973891 |
| Hmga2-Mmp9 | −0.01398601 | 0.973891 |
| Hmga2-Smad9 | −0.0036324 | 0.995863 |
| Hmga2-Inhbe | 0.04367131 | 1 |

TABLE 2
Spearman correlation R and p value associated with HMGA2 and TGF-β
signaling pathway genes in anti-PD-L1/TGFβ Trap treated animal group.

| Gene pair | Spearman R | P-value |
|---|---|---|
| Hmga2-Tert | 0.969409 | 2.62E−09 |
| Hmga2-Bmpr2 | 0.964286 | 7.82E−08 |
| Hmga2-Inhba | 0.942857 | 1.42E−07 |
| Hmga2-Acyr2a | 0.953571 | 2.80E−07 |
| Hmga2-Smad3 | 0.942857 | 7.96E−07 |
| Hmga2-Cdkn2b | −0.92143 | 1.07E−06 |
| Hmga2-Bmpr1a | 0.925 | 3.23E−06 |
| Hmga2-Zfyvc9 | 0.903571 | 3.88E−06 |
| Hmga2-Tgfbr2 | 0.910714 | 8.04E−06 |
| Hmga2-Mapk14 | 0.853571 | 5.17E−05 |
| Hmga2-Smad6 | 0.85 | 6.00E−05 |
| Hmga2-Dapk1 | 0.85 | 6.00E−05 |
| Hmga2-Inhbb | 0.846429 | 6.92E−05 |
| Hmga2-Acvr1 | 0.864286 | 7.40E−05 |
| Hmga2-Runx1 | 0.839286 | 9.14E−05 |
| Hmga2-Tgfb3 | 0.85 | 0.000126 |
| Hmga2-Smad7 | 0.828571 | 0.000135 |
| Hmga2-Pard6a | −0.82857 | 0.000135 |
| Hmga2-Smad7 | 0.828571 | 0.000135 |
| Hmga2-Tgfbr1 | 0.846429 | 0.000143 |
| Hmga2-Acyr1b | 0.842857 | 0.000162 |
| Hmga2-Foxk1 | 0.817857 | 0.000195 |
| Hmga2-Fos | 0.8 | 0.000342 |
| Hmga2-Mmp9 | −0.79643 | 0.00038 |
| Hmga2-Vegfa | 0.75 | 0.001281 |
| Hmga2-Col1a2 | 0.746429 | 0.001391 |
| Hmga2-Tgfbrap1 | 0.742857 | 0.001509 |
| Hmga2-Vim | 0.732143 | 0.001913 |
| Hmga2-Fn1 | 0.721429 | 0.002399 |
| Hmga2-Bmp7 | 0.732143 | 0.002677 |
| Hmga2-Col1a1 | 0.714286 | 0.002774 |
| Hmga2-Tgfb2 | 0.696429 | 0.005091 |
| Hmga2-Pja2 | 0.678571 | 0.005417 |
| Hmga2-Runx2 | 0.675 | 0.005763 |
| Hmga2-Bmp4 | 0.682143 | 0.006433 |
| Hmga2-Id1 | −0.65714 | 0.00777 |
| Hniga2-Bmp6 | 0.671317 | 0.007926 |
| Hmga2-Mmp2 | 0.646429 | 0.009215 |
| Hmga2-Bmp5 | 0.618284 | 0.016496 |
| Hmga2-Foxo4 | 0.589286 | 0.020794 |
| Hmga2-Twist2 | −0.58571 | 0.021777 |
| Hmga2-Tgfb1 | 0.589286 | 0.023175 |
| Hmga2-Col3a1 | 0.571429 | 0.026063 |
| Hmga2-Serpine1 | 0.571429 | 0.026063 |
| Hmga2-Bmpr1b | 0.575 | 0.027413 |
| Hmga2-Bmp2 | −0.56836 | 0.029259 |
| Hmga2-Zeb1 | 0.525 | 0.044484 |
| Hmga2-Acvr1c | 0.527046 | 0.046534 |
| Hmga2-Cdh | 0.514286 | 0.049839 |
| Hmga2-Jun | 0.507143 | 0.053664 |
| Hmga2-Gadd45b | −0.48929 | 0.06416 |
| Hmga2-Runx3 | 0.478571 | 0.071131 |
| Hmga2-Smad4 | 0.446429 | 0.095293 |
| Hmga2-Smad9 | 0.41654 | 0.122471 |
| Hmga2-Bmp10 | 0.347192 | 0.210989 |
| Hmga2-Itgb8 | 0.339286 | 0.216029 |
| Hmga2-Nodal | −0.29286 | 0.289472 |
| Hmga2-Gdf1 | −0.28666 | 0.300272 |
| Hmga2-Acyrl1 | 0.28597 | 0.301485 |
| Hmga2-Zeb2 | −0.28571 | 0.301936 |
| Hmga2-Smad1 | 0.271429 | 0.327789 |
| Hmga2-Foxo3 | 0.267857 | 0.334444 |
| Hmga2-Snai1 | 0.264286 | 0.341174 |
| Hmga2-Gdfl1 | 0.253571 | 0.361816 |
| Hmga2-Pja1 | 0.239286 | 0.390379 |
| Hmga2-Bmp3 | −0.23214 | 0.403941 |
| Hmga2-Foxo1 | 0.192857 | 0.491049 |
| Hmga2-Acw2b | −0.18929 | 0.498335 |
| Hmga2-Twist1 | 0.178571 | 0.524284 |
| Hmga2-Igf2 | −0.17143 | 0.541272 |
| Hmga2-Cdkn1a | −0.16071 | 0.567197 |
| Hmga2-Smad2 | 0.153571 | 0.584764 |
| Hmga2-Inha | 0.137623 | 0.624765 |
| Hmga2-Smad5 | −0.13571 | 0.62962 |
| Hmga2-Bmp15 | 0.127997 | 0.648966 |
| Hmga2-Dab2 | 0.110714 | 0.694463 |
| Hmga2-Itgb6 | −0.10357 | 0.71338 |
| Hmga2-Inhbe | −0.09798 | 0.728305 |
| Hmga2-Aldh1a1 | 0.085714 | 0.761334 |
| Hmga2-Snai2 | 0.085714 | 0.761334 |
| Hmga2-Stat3 | 0.067857 | 0.810109 |
| Hmga2-Sptbn1 | 0.064286 | 0.819948 |
| Hmga2-Il6 | −0.06071 | 0.829812 |
| Hmga2-Myc | −0.05714 | 0.8397 |
| Hmga2-Tgfbr3 | −0.05357 | 0.852484 |
| Hmga2-Cdh2 | 0.010714 | 0.96977 |
| Hmga2-Foxk2 | 0.010714 | 0.96977 |
| Hmga2-Bcl2l11 | −0.00714 | 0.979844 |
| Hmga2-Inhbc | No expression of Inhbc | No expression of Inhbc |

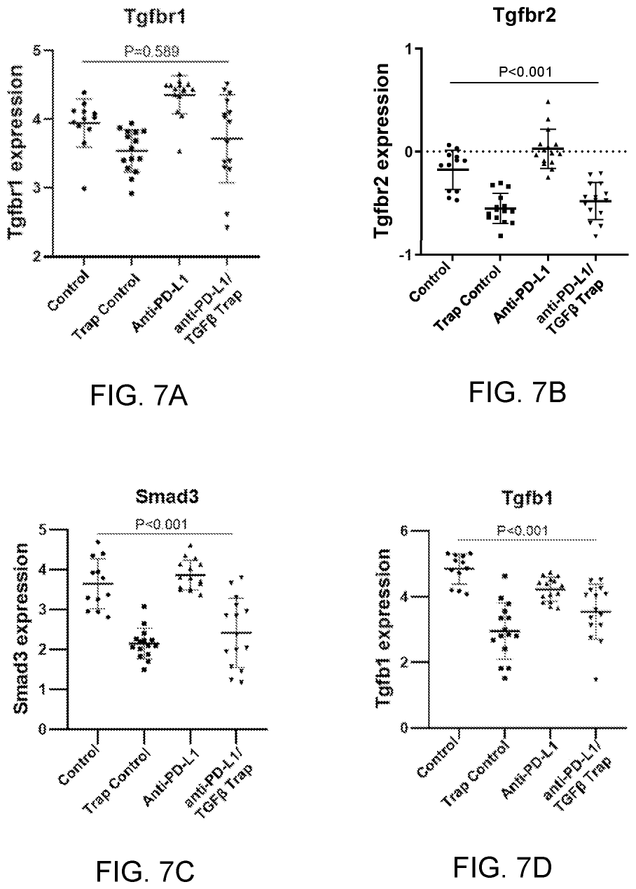

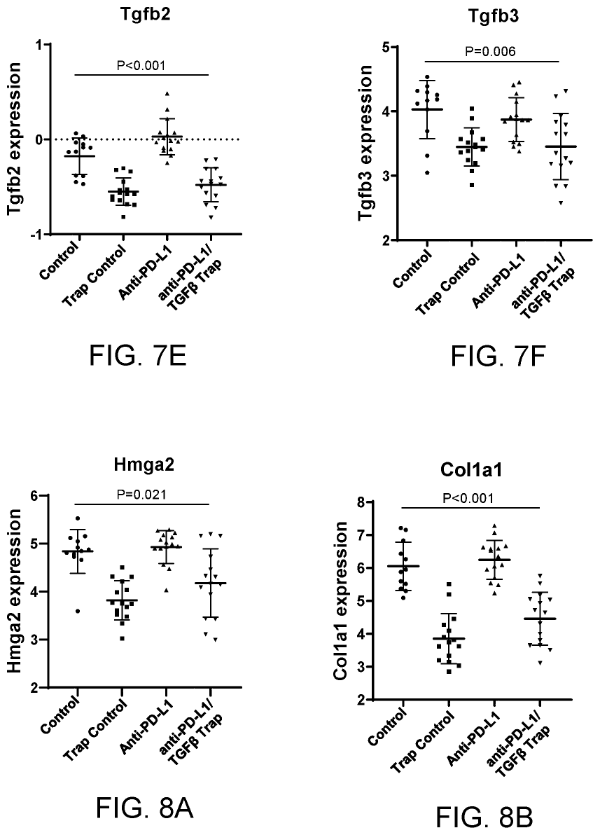

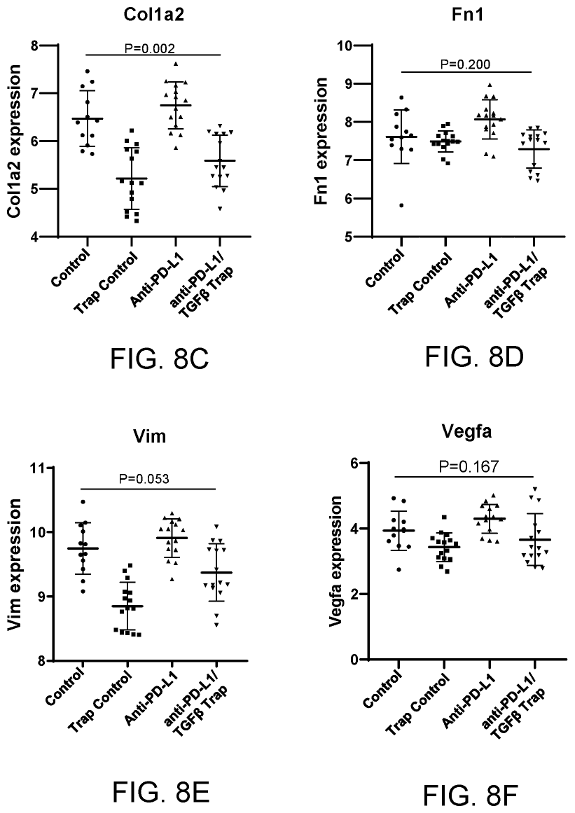

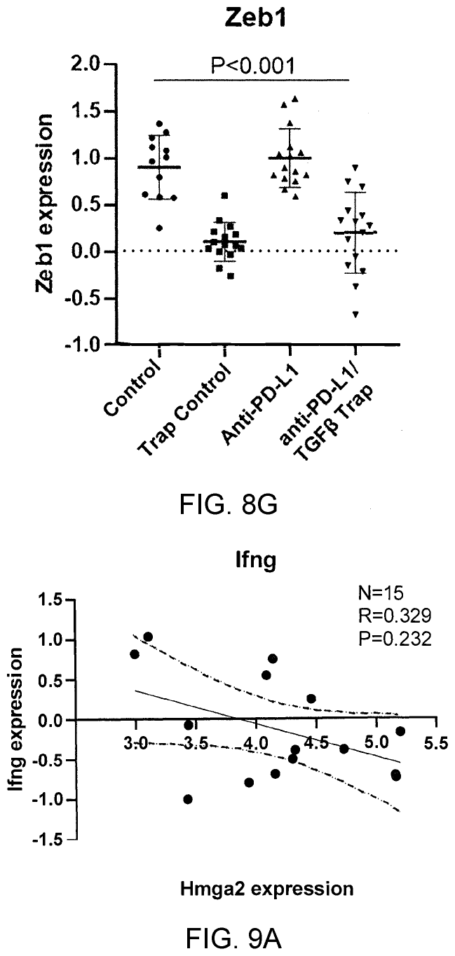

In addition to association studies, significant down regulation of HMGA2 and key TGF-β signaling core and target gene expression was observed in anti-PD-L1/TGFβ Trap-treated mice in comparison to control mice. FIGS. 7A-F are plots showing expression of TGF-β signaling genes in control, Trap control, anti-PD-L1, and anti-PD-L1/TGFβ Trap-treated animal groups. FIG. 7A is a plot showing expression of Tgfbr1 in control, Trap control, anti-PD-L1, and anti-PD-L1/TGFβ Trap-treated animal groups. FIG. 7B is a plot showing expression of Tgfbr2 in control, Trap control, anti-PD-L1, and anti-PD-L1/TGFβ Trap-treated animal groups. FIG. 7C is a plot showing expression of Smad3 in control, Trap control, anti-PD-L1, and anti-PD-L1/TGFβ Trap-treated animal groups. FIG. 7D is a plot showing expression of Tgfb1 in control, Trap control, anti-PD-L1, and anti-PD-L1/TGFβ Trap-treated animal groups. FIG. 7E is a plot showing expression of Tgfb2 in control, Trap control, anti-PD-L1, and anti-PD-L1/TGFβ Trap-treated animal groups. FIG. 7F is a plot showing expression of Tgfb3 in control, Trap control, anti-PD-L1, and anti-PD-L1/TGFβ Trap-treated animal groups. These plots show that anti-PD-L1/TGFβ Trap-treated animal group showed a downregulation in expression of Tgfbr1, Tgfbr2, Smad3, Tgfb1, Tgfb2, and Tgfb3. FIGS. 8A-G are plots showing expression of key TGF-β target genes in control, Trap control, anti-PD-L1, and anti-PD-L1/TGFβ Trap-treated animal groups. FIG. 8A is a plot showing expression of HMGA2 in control, Trap control, anti-PD-L1, and anti-PD-L1/TGFβ Trap-treated animal groups. FIG. 8B is a plot showing expression of Col1a1 in control, Trap control, anti-PD-L1, and anti-PD-L1/TGFβ Trap-treated animal groups. FIG. 8C is a plot showing expression of Col1a2 in control. Trap control, anti-PD-L1, and anti-PD-L1/TGFβ Trap-treated animal groups. FIG. 8D is a plot showing expression of Fn1 in control, Trap control, anti-PD-L1, and anti-PD-L1/TGFβ Trap-treated animal groups. FIG. 8E is a plot showing expression of Vim in control, Trap control, anti-PD-L1, and anti-PD-L1/TGFβ Trap-treated animal groups. FIG. 8F is a plot showing expression of Vegfa in control, Trap control, anti-PD-L1, and anti-PD-L1/TGFβ Trap-treated animal groups. FIG. 8G is a plot showing expression of Zeb1 in control, Trap control, anti-PD-L1, and anti-PD-L1/TGFβ Trap-treated animal groups. FIGS. 8A-G show that anti-PD-L1/TGFβ Trap treated animal group showed a downregulation in expression of HMGA2 and TGF-β target genes Col1a1, Col1a2, Fn1, Vim, Vegfa, and Zeb1, respectively.

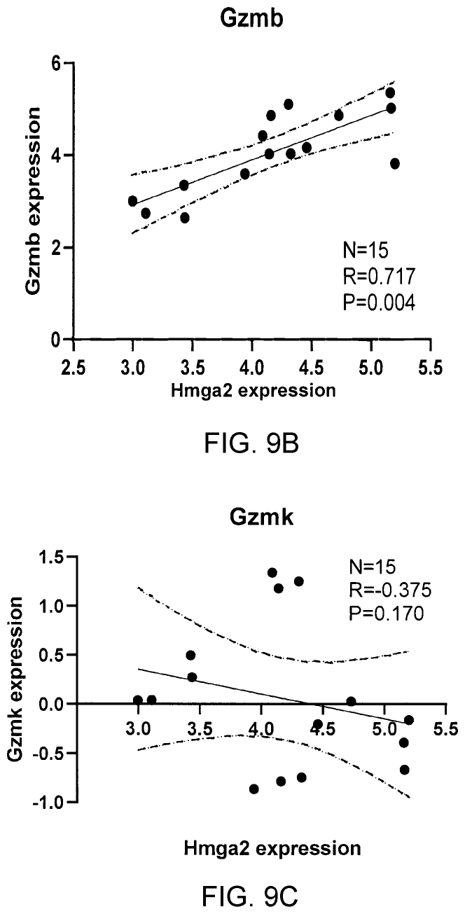

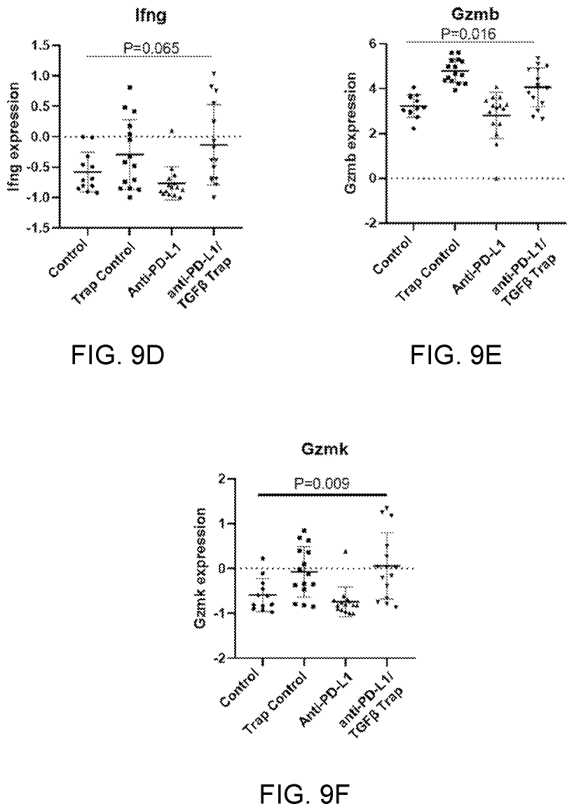

Spearman correlation analysis was also performed on HMGA2 and a PD-1/IFNγ (Interferon gamma) signaling signature (see M. Ayers et al., J. Clin. Invest. 127, 2930-2940 (2017)) which showed only 11% (2/18) and 17% (3/18) of the PD-1/IFNγ signaling gene pairs have a statistically significant R in the control and anti-PD-L1/TGFβ Trap treatment group, respectively. Table 3 lists Spearman correlation R and p value associated with gene pairs in control animal group. Table 4 lists Spearman correlation R and p value associated with gene pairs in anti-PD-L1/TGFβ Trap treatment group. FIGS. 9A-C are scatter plots showing association between HMGA2 and selected PD-1/IFNγ related genes Ifng, Gzmb, and Gzmk, respectively. FIGS. 9D-F show expression levels of selected PD-1/IFNγ related genes Ifng, Gzmb, and Gzmk in control, trap control, Anti-PD-L1, and anti-PD-L1/TGFβ Trap-treated animals, respectively. FIG. 9A is a scatter plot showing association between HMGA2 expression and Ifng expression. FIG. 9B is a scatter plot showing association between HMGA2 expression and Gzmb expression. FIG. 9C is a scatter plot showing association between HMGA2 expression and Gzmk expression. FIG. 9D is a plot showing expression of Ifng in control, trap control, Anti-PD-L1, and anti-PD-L1/TGFβ Trap-treated animals. FIG. 9E is a plot showing expression of Gzmb in control, trap control, Anti-PD-L1, and anti-PD-L1/TGFβ Trap-treated animals. FIG. 9F is a plot showing expression of Gzmk in control, trap control, Anti-PD-L1, and anti-PD-L1/TGFβ Trap-treated animals.

TABLE 3
Spearman correlation R and p value associated with HMGA2
and PD-1/IFNγ signaling pathway genes in control animal group.

| Gene pair | Spearman R | P-value |
|---|---|---|
| Hmga2-Cxcr6 | −0.66434 | 0.021901 |
| Hmga2-Gzmk | −0.59441 | 0.045705 |
| Hmga2-Ifng | −0.49387 | 0.105021 |
| Hmga2-Cxcl9 | −0.3986 | 0.200954 |
| Hmga2-Tagap | −0.39161 | 0.209711 |
| Hmga2-Cd3d | −0.36364 | 0.246411 |
| Hmga2-Nkg7 | −0.32867 | 0.297333 |
| Hmga2-Ccl5 | −0.3007 | 0.342415 |
| Hmga2-Stat1 | −0.23077 | 0.470802 |
| Hmga2-Lag3 | −0.1958 | 0.543064 |
| Hmga2-Gzmb | 0.174825 | 0.588091 |
| Hmga2-Cd2 | −0.13287 | 0.68316 |
| Hmga2-Cxcl10 | −0.11888 | 0.71599 |
| Hmga2-Ciita | −0.1049 | 0.749263 |
| Hmga2-Ido1 | −0.08392 | 0.800385 |
| Hmga2-Il2rg | 0.013986 | 0.973891 |
| Hmga2-Cd3e | 0.006993 | 0.991026 |
| Hmga2-Cxcl13 | −0.00699 | 0.991026 |

TABLE 4
Spearman correlation R and p value associated with HMGA2 and PD-1/IFNγ
signaling pathway genes in anti-PD-L1/TGFβ Trap treated animal group.

| Gene pair | Spearman R | P-value |
|---|---|---|
| Hmga2-Lag3 | −0.89643 | 1.76E−05 |
| Hmga2-Cd3d | −0.875 | 4.78E−05 |
| Hmga2-Gzmb | 0.717857 | 0.0035 |
| Hmga2-Nkg7 | −0.47857 | 0.073469 |
| Hmga2-Ccl5 | −0.45219 | 0.091978 |
| Hmga2-Cd2 | −0.39286 | 0.148547 |
| Hmga2-Tagap | 0.389286 | 0.152504 |
| Hmga2-Gzmk | −0.375 | 0.169155 |
| Hmga2-Ifng | −0.32857 | 0.231684 |
| Hmga2-Cxcl10 | 0.3 | 0.276736 |
| Hmga2-Cxcr6 | −0.26988 | 0.328113 |
| Hmga2-Stat1 | −0.25357 | 0.360726 |
| Hmga2-Ciita | 0.253571 | 0.360726 |
| Hmga2-Cxcl9 | −0.24643 | 0.374827 |
| Hmga2-Ido1 | 0.235714 | 0.396592 |
| Hmga2-Cxcl13 | −0.20714 | 0.457799 |
| Hmga2-Cd3e | −0.20357 | 0.465738 |
| Hmga2-Il2rg | 0.175 | 0.532005 |

Table 5 provides a summary of gene expression data for TGF-β signaling genes in control, trap control, anti-PD-L1, and anti-PD-L1/TGFβ Trap-treated animal groups. Table 6 provides a summary of gene expression data for PD-1/IFNγ signaling genes in control, trap control, anti-PD-L1, and anti-PD-L1/TGFβ Trap-treated animal groups.

TABLE 5
Summary of gene expression data for TGF-β signaling genes
in control, trap control, anti-PD-L1, and anti-PD-L1/TGFβ
Trap-treated animal groups.

| | | | | | P-value | | |
|---|---|---|---|---|---|---|---|
| | | | | | Trap | | anti-PD- |
| | Average Log2 TPM | | | anti-PD-L1/ | control | Anti-PD-L1 | L1/TGFβ |
| | | Trap | | TGFβ | vs | vs | Trap vs |
| Genes | Control | Control | Anti-PD-L1 | Trap | Control | Control | Control |
| Co11a1 | 6.052 | 3.855 | 6.251 | 4.462 | <0.0001 | 0.8099 | <0.0001 |
| Dapk1 | 1.356 | 0.465 | 1.353 | 0.541 | <0.0001 | >0.9999 | <0.0001 |
| Foxk1 | 1.751 | 0.442 | 1.853 | 0.55 | <0.0001 | 0.9109 | <0.0001 |
| Mapk14 | 3.542 | 2.672 | 3.744 | 2.766 | <0.0001 | 0.3943 | <0.0001 |
| Runx1 | 2.578 | 1.622 | 2.772 | 1.856 | <0.0001 | 0.3289 | <0.0001 |
| Runx2 | 1.636 | 0.494 | 1.95 | 0.891 | <0.0001 | 0.1418 | <0.0001 |
| Runx3 | 1.878 | 0.438 | 1.621 | 0.841 | <0.0001 | 0.3667 | <0.0001 |
| Smad7 | 3.155 | 2.296 | 3.016 | 2.243 | <0.0001 | 0.6174 | <0.0001 |
| Tert | 0.113 | −0.621 | 0.092 | −0.57 | <0.0001 | 0.9956 | <0.0001 |
| Zeb1 | 0.901 | 0.098 | 0.992 | 0.188 | <0.0001 | 0.81 | <0.0001 |
| Pard6a | 2.292 | 3.477 | 2.099 | 3.216 | <0.0001 | 0.6598 | 0.0001 |
| Il6 | 1.338 | 2.457 | 1.938 | 2.532 | 0.0007 | 0.0938 | 0.0003 |
| Co11a2 | 6.472 | 5.217 | 6.747 | 5.589 | <0.0001 | 0.4436 | 0.0005 |
| Snai1 | 3.643 | 3.2 | 3.302 | 3.145 | 0.0042 | 0.0334 | 0.0012 |
| Gadd45b | 3.166 | 3.559 | 2.987 | 4.005 | 0.214 | 0.7653 | 0.0017 |
| Hmga2 | 4.838 | 3.818 | 4.929 | 4.177 | <0.0001 | 0.9325 | 0.0036 |
| Foxk2 | 3.308 | 3.293 | 3.384 | 3.098 | 0.9899 | 0.4989 | 0.0061 |
| Pja2 | 3.399 | 2.941 | 3.735 | 3.019 | 0.001 | 0.0179 | 0.0066 |
| Mmp2 | 3.883 | 2.297 | 3.956 | 3.113 | <0.0001 | 0.9805 | 0.0084 |
| Itgb8 | −0.526 | −0.436 | −0.274 | −0.258 | 0.5952 | 0.0156 | 0.0094 |
| Foxo1 | 2.008 | 1.557 | 2.046 | 1.567 | 0.009 | 0.9864 | 0.0108 |
| Cdh1 | 3.996 | 3.454 | 4.124 | 3.403 | 0.0227 | 0.8473 | 0.0116 |
| Foxo4 | 1.914 | 0.865 | 2 | 1.419 | <0.0001 | 0.9242 | 0.0194 |
| Foxo3 | 1.771 | 1.243 | 1.973 | 1.469 | <0.0001 | 0.1843 | 0.0269 |
| Id1 | 4.659 | 5.146 | 4.23 | 5.02 | 0.0018 | 0.0066 | 0.0274 |
| Vim | 9.749 | 8.852 | 9.909 | 9.374 | <0.0001 | 0.5559 | 0.0363 |
| Jun | 4.145 | 4.137 | 4.244 | 3.896 | 0.9997 | 0.6829 | 0.0693 |
| Stat3 | 4.412 | 3.945 | 4.59 | 4.137 | 0.003 | 0.4055 | 0.1125 |
| Twist2 | 1.86 | 1.557 | 1.219 | 1.538 | 0.2471 | 0.0031 | 0.2059 |
| Dab2 | 4.888 | 4.688 | 4.958 | 4.632 | 0.4165 | 0.938 | 0.2315 |
| Fn1 | 7.615 | 7.492 | 8.069 | 7.295 | 0.8605 | 0.0636 | 0.25 |
| Fos | 2.637 | 2.354 | 3.037 | 2.349 | 0.288 | 0.0852 | 0.2756 |
| Igf2 | −0.433 | 0.064 | −0.432 | −0.15 | 0.0287 | >0.9999 | 0.304 |
| Mmp9 | 4.929 | 5.866 | 4.716 | 5.402 | 0.0119 | 0.8309 | 0.305 |
| Cdkn1a | 4.02 | 3.206 | 4.364 | 4.221 | <0.0001 | 0.0396 | 0.323 |
| Vegfa | 3.936 | 3.43 | 4.3 | 3.665 | 0.0722 | 0.2518 | 0.4752 |
| Scrpinc1 | 4.932 | 4.214 | 5.642 | 4.658 | 0.0172 | 0.0185 | 0.5561 |
| Co13a1 | 7.679 | 7.78 | 7.877 | 7.471 | 0.9277 | 0.6598 | 0.6258 |
| Cdh2 | −0.605 | −0.764 | −0.541 | −0.661 | 0.0296 | 0.5672 | 0.6682 |
| Itgb6 | −0.748 | −0.784 | −0.589 | −0.68 | 0.9248 | 0.0921 | 0.6801 |
| Zeb2 | 2.51 | 2.349 | 2.999 | 2.663 | 0.6715 | 0.0192 | 0.6998 |
| Twist1 | 3.221 | 3.299 | 3.04 | 3.152 | 0.8229 | 0.2583 | 0.8643 |
| Pja1 | 4.26 | 4.317 | 4.159 | 4.213 | 0.8141 | 0.4533 | 0.8826 |
| Bcl2111 | 3.551 | 2.906 | 3.772 | 3.447 | 0.0037 | 0.5005 | 0.8983 |
| Snai2 | 0.38 | 0.27 | 0.411 | 0.308 | 0.7796 | 0.9929 | 0.921 |
| Cdkn2b | 0.852 | 0.819 | 0.605 | 0.899 | 0.9925 | 0.2543 | 0.9783 |
| Myc | 4.979 | 5.669 | 4.854 | 4.948 | <0.0001 | 0.5452 | 0.9851 |
| Aldh1a1 | −0.338 | −0.633 | 0.004 | −0.369 | 0.3836 | 0.273 | 0.9976 |

TABLE 6
Summary of gene expression data for PD-1/IFNγ signaling genes in
control, trap control, anti-PD-L1, and anti-PD-Ll/TGFβ Trap-treated animal groups.

| | | | | | P-value | | |
|---|---|---|---|---|---|---|---|
| | Genes Average Log2 TPM | | | | | | anti-PD- |
| | | | | anti-PD- | | | L1/TGFβ |
| | | Trap | | L1/TGFβ | Trap control | Anti-PD-L1 | Trap vs |
| | Control | Control | Anti-PD-L1 | Trap | vs Control | vs Control | Control |
| Lag3 | 1.037 | 2.018 | 0.705 | 1.988 | 0.0008 | 0.4053 | 0.0012 |
| Cd3d | 2.059 | 4.733 | 0.964 | 4.241 | 0.0001 | 0.1767 | 0.0019 |
| Nkg7 | 2.334 | 3.313 | 1.442 | 3.415 | 0.0103 | 0.021 | 0.0042 |
| Gzmk | −0.589 | −0.072 | −0.74 | 0.057 | 0.0395 | 0.7935 | 0.0078 |
| Gzmb | 3.227 | 4.786 | 2.804 | 4.064 | <0.0001 | 0.3584 | 0.0203 |
| Cxcl9 | 2.124 | 2.701 | 1.325 | 3.486 | 0.5039 | 0.2542 | 0.022 |
| Ciita | 1.142 | 0.075 | 0.511 | 0.384 | 0.001 | 0.0705 | 0.0237 |
| Ifng | −0.582 | −0.292 | −0.766 | −0.134 | 0.2953 | 0.6422 | 0.057 |
| Stat1 | 3.554 | 4.087 | 3.307 | 4.077 | 0.0706 | 0.5849 | 0.0777 |
| Cxcr6 | 0.817 | 1.92 | 0.302 | 1.379 | 0.0002 | 0.1156 | 0.0777 |
| Cxcl10 | 3.398 | 2.546 | 3.056 | 2.898 | 0.0206 | 0.5388 | 0.249 |
| Idol | −0.119 | 0.055 | −0.225 | 0.262 | 0.791 | 0.9394 | 0.2522 |
| Cd2 | 1.663 | 2.027 | 1.069 | 2.47 | 0.8246 | 0.5343 | 0.2983 |
| Il2rg | 3.545 | 2.191 | 3.208 | 3.003 | 0.0015 | 0.6707 | 0.3173 |
| Tagap | 1.433 | 1.24 | 1.27 | 1.249 | 0.4463 | 0.5751 | 0.4856 |
| Ccl5 | 4.516 | 4.648 | 3.682 | 4.941 | 0.9738 | 0.103 | 0.5705 |
| Cd3e | 1.133 | 0.42 | 0.326 | 0.875 | 0.3522 | 0.2596 | 0.9147 |
| Cxcl13 | 1.159 | 0.888 | 0.776 | 1.216 | 0.9634 | 0.907 | 0.9996 |

Overall, HMGA2 expression had a stronger association with TGF-β signaling in the anti-PD-L1/TGFβ Trap treatment group when compared to the control treatment group based on the magnitude of Spearman R and the number of gene pairs with statistically significant P-values, indicating that HMGA2 expression is responsive to TGF-β specific transcriptomic changes induced by anti-PD-L1/TGFβ Trap treatment. The lower percentage of statistically significant association between HMGA2 and PD-1 blockade response signature shows that HMGA2 expression is less indicative of the changes in immune related genes. The data illustrates that HMGA2 expression is indicative of TGF-β signaling activity and hence can be used as a stratification and/or treatment response biomarker.

### Example 3: Methods for Identifying TNBC Patients Likely to Respond to an Anti-PD-L1/TGFβ Trap Protein Therapy

RT-qPCR is a semi-quantitative method to analyze the gene expression of a target and is one method used to determine HMGA2 gene expression level. Alternatively, digital droplet PCR (ddPCR), which allows for absolute quantification in copies of the target in a given sample, is used to determine HMGA2 gene expression level. Another alternative assay to determine HMGA2 gene expression level is the HTG EdgeSeq NGS technology, which is a targeted RNA-Sequencing based on a quantitative nuclease protection chemistry that enables extraction-free quantitation of mRNA/miRNAs from FFPE tissue and a variety of other sample types and can offer broaden pathway coverage of HMGA2 and upstream/downstream markers. The assay acceptance criteria include specificity, robustness, sensitivity (LOD & LOQ), efficiency & linearity, precision (Repeatability) and the intra- & inter-assay variability. Once assay set up, analytical validation, clinical validation will be performed in CLIA/CAP certified laboratory.

### RT-qPCR

RT-qPCR is a semi-quantitative method to analyze the gene expression of a target relative to the expression level of a house-keeping gene. There are numerous Taqman qPCR assays for HMGA2 as well as Bio-Rad PrimePCR assays for HMGA2 which are commercially available. A set of primers/probes, which ensures linearity of the assay and efficiency of the primer/probe set using a synthetic construct (SEQ ID NO: 65), which spans all regions covered in each assay, is used to determine HMGA2 RNA expression level in samples obtained from TNBC patients. Biological samples are tested using cDNA converted from RNA extracted from a cell line with high expression of HMGA2 (e.g., breast cancer cell lines e.g., SW480 or MCF7, transfected with HMGA2)) and cDNA converted from RNA extracted for FFPE samples from patients with TNBC.

Synthetic construct is provided in SEQ ID NO: 65

GCGAAGCGGCTGCAGCGGCGGTAGCGGCGGCGGGAGGCAGGATGAGCGCA
CGCGGTGAGGGCGCGGGGCAGCCGTCCACTTCAGCCCAGGGACAACCTGC
CGCCCCAGCGCCTCAGAAGAGAGGACGCGGCCGCCCCAGGAAGCAGCAGC
AAGAACCAACCGGTGAGCCCTCTCCTAAGAGACCCAGGGGAAGACCCAAA
GGCAGCAAAAACAAGAGTCCCTCTAAAGCAGCTCAAAAGAAAGCAGAAGC
CACTGGAGAAAAACGGCCAAGAGGCAGACCTAGGAAATGGCCACAACAAG
TTGTTCAGAAGAAGCCTGCTCAGGTCAATGTTGCCTTGCCTGGGAAGGAC
CACCCGGGCAATCTTATATATCTACTGTTCTCTAAA

### Digital Droplet PCR

Digital Droplet PCR (ddPCR) allows for absolute quantification in copies of the target in a given sample. This is a distinct advantage over qPCR however can be more difficult to implement in a clinical setting. However, for tissue samples with low HMGA2 tissue abundancy, ddPCR is considered to determine the HMGA2 expression level. Each assay is compared using a dilution of cDNA constructs, patient derived cDNA, and cDNA from cell lines which are either positive or negative for HMGA2.

### HTG EdgeSeq

The HTG EdgeSeq NGS technology is a targeted RNA-Sequencing, which is based on a quantitative nuclease protection chemistry that enables extraction-free quantitation of mRNA/miRNAs from FFPE tissue and a variety of other sample types. The chemistry significantly reduces sample input requirements compared to standard RNA-Sequencing. The combination of low sample input and simplified workflow makes HTG EdgeSeq NGS an appealing technology for clinical applications. The panel is available off-the-shelf and is used to broaden pathway coverage of HMGA2 and upstream/downstream markers.

### Analysis of RNA-Seq

Because RNA-seq provides only relative RNA abundance, a cutoff relative to a population average was determined as follows: first, a large RNA-seq dataset (TCGA) was examined, and the population median expression for HMGA2 and MECOM were obtained from the database. The separation factor separating this population median of the database from the lowest expression among the responders of the current study was ascertained to be 2.27 for HMGA2 and 1.54 for MECOM.

Having determined these factors using RNA-seq, an absolute cutoff was determined by first choosing a gene expression assay with absolute (rather than relative) quantitation of expression, measuring the median HMGA2 and MECOM expression, respectively, among TNBC patients using this assay, then multiplying that median by the respective separation factor.

After batch correction with ComBat, the lowest HMGA2 expression among the responders was found to be 0.700 log-TPM. The median expression of HMGA2 in TCGA-BRCA-TNBC was −0.483. A difference of 1.18 in log-2 scale corresponds to a separation factor of 2.27. In other words, patients whose HMGA2 expression is at least 2.27 times higher than the population mean among TNBC patients were likely to respond to anti-PD-L1-TGFβ treatment. For MECOM, the lowest expression among the responders was 2.35 log-TPM, while the median expression in TCGA-BRCA-TNBC was 1.73 log-TPM, a separation factor of 1.54, suggesting that patients whose MECOM expression is at least 1.54 times higher than the population mean among TNBC patients were likely to respond to anti-PD-L1-TGFβ treatment.

### Example 4: Method for Determining HMGA2 Levels in Samples Obtained from TNBC Patients Treated with an Anti-PD-L1/TGFβ Trap Protein

This example relates to a method for determining HMGA2 levels in samples obtained from TNBC patients for ascertaining the responsiveness to an anti-PD-L1/TGFβ protein therapy. Quantitative real-time PCR, digital droplet PCR and HTG EdgeSeq system were used for the detection of High-mobility group AT-hook 2 (HMGA2) using human FFPE samples.

RNA extraction: Triple negative breast cancer (TNBC) formalin fixed paraffin-embedded (FFPE) samples were procured from various commercial sources (tumor percentage range of 25-100%). FFPE blocks were cut into 5 pM sections and collected individually in tubes. RNA was extracted from two FFPE curls for each sample using the Qiagen RNeasy FFPE kit (Product #73504). The two aliquots for each sample remained separate until the sample was added the membrane spin columns. This allowed for more complete melting of paraffin but also to concentrate the samples. Samples were eluted from the spin column in 30 μL of water.

### RNA Quantification

Following extraction, RNA was quantified using the Qubit RNA Broad Range Assay Kit (Product #Q10211). Extracted RNA was diluted 1:66.67 (3 μL RNA+197 μL Qubit working solution). If a sample was outside the acceptable range as considered as the control in the Qubit assay, sample was diluted further, or a lower dilution was used as needed to produce a reliable concentration reading.

### RNA Quality Assessment

RNA samples were also run on the Agilent Bioanalyzer platform to assess the quality of the extracted RNA using the Agilent RNA 6000 Nano kit (Product #5067-1512).

### Reverse Transcription

cDNA was transcribed from template RNA using the Invitrogen/ThermoFisher Superscript IV VILO Master Mix (Product #11766050). 100 ng of RNA was used for each reaction. Per the manufacturer's protocol, 4 μL of the Superscript IV VILO Master mix was added to each reaction along with enough nuclease free water to bring the total volume to 20 μL. Multiple reactions for each sample could be combined to convert as much RNA to cDNA as possible in one reaction. In cases where the concentration of RNA was too low, transcription reactions were made at the greatest possible concentration. All reactions mixtures were then incubated at 25° C. for 15 minutes, then 50° C. for 15 minutes, and finally 85° C. for 10 minutes. cDNA was then stored at −20° C. until ready for use.

qPCR was performed using the Applied Biosystems 7500Dx instrument. Briefly, qPCR mixes consisted of 10 μL of Taqman 2× Gene expression master mix from Thermo Fisher (Product #4369016), 1 μL of either Taqman HMGA2 Primer Probe Set Hs0017569_m1 (SEQ ID NO: 63 and/or SEQ ID NO: 64) or Taqman ACTB Primer Probe Set Hs01060665_g1, 4 μL cDNA from samples, and 5 μL of H₂O for a total reaction volume of 20 μL. Primer/probe set for target genes (SEQ ID NO: 63 and/or SEQ ID NO: 64) and house-keeping genes can be designed using Primer Express® if “off-the-shelf” gene expression assay is not available. qPCR was then ran with the following protocol: Hold at 50° C. for 2 minutes, hold at 95° C. for 10 minutes, 40 cycles of 95° C. for 15 seconds followed by 60° C. for 1 minute. Thresholding was performed using the auto analysis function of the software.

qPCR Analysis:

The comparative delta Ct (ΔCt) method was used for relative quantification of gene expression. qPCR sample analysis was performed looking at both raw HMGA2 cycle threshold (Ct) values and housekeeping gene Ct values as well Delta Ct values calculated as (Ct value of HMGA2−Ct value of housekeeping gene). In an exemplary embodiment, ACTB (Beta Actin) may be used as a housekeeping gene. In an exemplary embodiment, delta Ct values may be calculated as (Ct value of HMGA2−Ct value of ACTB). In certain embodiments, more than one housekeeping gene can be used and Ct values obtained from housekeeping genes can be averaged. In certain embodiments, delta Ct values may be calculated as (Ct value of HMGA2−average Ct value of one or more housekeeping genes). A lower ΔCt value or lower raw Ct values signifies a higher HMGA2 expression.

Digital Droplet PCR (ddPCR)

To confirm the initial qPCR results, ddPCR as an orthogonal method was performed on the same samples but using different primer/probe sets which were more appropriate to the ddPCR application. 22 μL of ddPCR reaction mixes were made consisting of the following: 11 μL of Bio-Rad ddPCR Supermix for Probes (Product #186-3026), 1 μL of HMGA2 Bio-Rad ddPCR assay ID: dHSaCPE5029086 FAM probe, 1 μL ACTB Bio-Rad ddPCR assay ID: dHsaCPE5190200 HEX probe, 4 μL cDNA from samples, and 5 μL of H₂O. Droplets were generated in a Bio-Rad AutoDG instrument and then amplified in VeritiDx Thermal Cycler with the following conditions: Hold 95° C. for 10 minutes, 40 cycles of 94° C. for 30 seconds then 60° C. for 1 minute, hold 98° C. for 10 minutes, hold 4° C. for at least 30 minutes. After amplification, PCR reactions were transferred to a Bio-Rad QX200 plate reader and droplets were analyzed. Thresholds were set manually for each sample to differentiate the positive droplets from the negative droplets for each sample.

ddPCR Analysis:

Sample analysis of each experiment is performed using QuantaSoft software. Positive droplet concentrations in all samples were determined using manually placed fluorescence thresholds based on negative clusters as detected in the corresponding no template control (NTCs). Target DNA concentration (copies/μL) and absolute droplet counts within single samples were used as quantitative outcome measurement. ddPCR provides absolute quantification as copies/well (reaction), and therefore higher ddPCR ratio values correspond with higher HMGA2 expression. ddPCR sample analysis was performed looking at both raw HMGA2 copy number values as well HMGA2 copy number values that have been normalized to copy number values obtained from one or more housekeeping genes. Normalized copy number values are calculated as (Copy number value of HMGA2/copy number value of single (or average) housekeeping gene). In an exemplary embodiment, ACTB (Beta Actin) may be used as a housekeeping gene.

### HTG EdgeSeq:

The FFPE specimens were scraped into tubes and lysed in HTG's lysis buffer, followed by the introduction of gene-specific DNA nuclease protection probes (NPP). After allowing the NPPs to hybridize to their target RNAs, which can be both soluble or cross-linked in the biological matrix, S1 nuclease is added which removes excess unhybridized NPPs and RNAs, leaving behind only NPPs hybridized to their target RNAs. Thus, a stoichiometric conversion of the target RNA to the NPPs is achieved, producing a virtual 1:1 ratio of NPP to RNA. The qNPA steps are automated on the HTG EdgeSeq processor, which is followed by PCR to add sequencing adaptors and tags. The labeled samples are pooled, cleaned, and sequenced on a next generation sequencing (NGS) platform using standard protocols. Data from the NGS instrument are processed and reported by the HTG EdgeSeq parser software.

Selection of Patient Population for Treatment with Anti-PD-L1/TGFβ Trap Based on HMGA2 Expression

Data presented in Example 1 showed that there is a significant over-expression of HMGA2 in TNBC patients who responded to anti-PD-L1/TGFβ Trap treatment (responders) compared to TNBC patients who did not respond to anti-PD-L1/TGFβ Trap protein (non-responders). This section illustrates methods of selecting patients for treatment with anti-PD-L1/TGFβ by using cutoffs (e.g., Ct values or count levels) that signify high HMGA2 expression. In certain embodiments, HMGA2 high expression cutoff to select patient population that will respond to anti-PD-L1/TGFβ Trap protein treatment is deduced by incorporation of data obtained from RNA-seq and data obtained from qPCR and/or ddPCR. The TPM values obtained from RNA-seq may be translated into quantitation values that can be obtained from absolute quantitation methods (e.g., qPCR or ddPCR). A transfer function that maps from TPM values obtained from RNA-seq to Ct values (for qPCR) or ddPCR ratio values (for ddPCR) is generated. This transfer function is used to find the corresponding Ct or ddPCR ratio levels that can provide a cutoff with regards to high HMGA2 expression. In certain embodiments, transfer function used to find corresponding Ct values that can provide a cutoff with regards to high HMGA2 expression is:

Y₁=X₁−log 2(TPM_{lowest}/TPM_{baseline});

where Y₁=Ct value cutoff;

X₁=normalized ΔCt value (median relative qPCR expression for HMGA2);

TPM_{lowest}=lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In certain embodiments, transfer function used to find corresponding Ct values that can provide a cutoff with regards to high HMGA2 expression is:

Y₁=X₁−log 2(TPM_{second lowest}/TPM_{baseline});

where Y₁=Ct value cutoff;

X₁=normalized ΔCt value (median relative qPCR expression for HMGA2);

TPM_{second lowest}=lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In certain embodiments, transfer function used to find corresponding ddPCR ratio values that can provide a cutoff with regards to high HMGA2 expression is:

Y₁=X₁×(TPM_{lowest}/TPM_{baseline});

where Y₁=ddPCR ratio value cutoff;

X₁=normalized ddPCR ratio value (median ddPCR ratio value for HMGA2);

TPM_{lowest}=lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In certain embodiments, transfer function used to find corresponding ddPCR ratio values that can provide a cutoff with regards to high HMGA2 expression is:

Y₁=X₁×(TPM_{second lowest}/TPM_{baseline});

where Y₁=ddPCR ratio value cutoff;

X₁=normalized ddPCR ratio value (median ddPCR ratio value for HMGA2);

TPM_{second lowest}=second lowest HMGA2 expression (TPM value) obtained from RNA-seq among patients that respond to anti-PD-L1/TGFβ Trap protein treatment;

TPM_{baseline}=median HMGA2 expression among all patients regardless of clinical response.

In certain embodiments, a set of tumor samples of sufficient size (e.g., 50, 100, 150, 200, or 250 tumor samples) is obtained, and RNA-seq and qPCR (or ddPCR) is performed on each sample to produce a matched dataset that allows for a direct comparison of HMGA2 expression quantitation by RNA-seq and qPCR. A model transfer function from qPCR Ct value (or ddPCR ratio value) may be obtained by performing spline regression modeling expression level as a function of TPM (see Friedman, Jerome H. “Multivariate adaptive regression splines.” Annals of Statistics 19.1 (1991): 1-67; see also Kuhn, Max, and Kjell Johnson. APPLIED PREDICTIVE MODELING. Vol. 26. New York: Springer, 2013). The validity of the transfer function and magnitude of any overall batch effects is tested by comparing the distribution of HMGA2 in the new dataset to that obtained from the data set described in Example 1. Once it is established that the batch effects do not impair the utility of this model transfer function, then this transfer function will be utilized to obtain high HMGA2 expression cutoffs.

HMGA2 Expression Data from RNA-seq (Fold-Change Derivation Method): In an exemplary population cohort of TNBC patients treated with anti-PD-L1/TGFβ Trap protein, formalin-fixed, paraffin embedded (FFPE) tumor samples (n=118) are used to extract RNA and assess the quality of RNA as described in Example 4. Analysis of RNA-seq (as described in Example 1) performed on samples that passed the quality control (n=103) shows a mean difference of 32-fold in HMGA2 expression in responders vs non-responders. The same exemplary population cohort shows median expression of HMGA2 among all patients regardless of clinical response as 9.82 transcripts per million (TPM), lowest HMGA2 expression among responders as 18.86 TPM, and second-lowest HMGA2 expression among responders as 177.75 TPM.

HMGA2 Expression Cutoff using Ct Values (Fold-Change Derivation Method): In parallel, qPCR experiment is performed on all samples (n=103) that passed the quality control using methods described in Example 4 to obtain expression Ct values for HMGA2 and a housekeeping gene, beta-actin. Using the analysis method (comparative ΔCt method) for qPCR experiment described in Example 4, a ΔCt of 12.1 (median relative qPCR expression for HMGA2) is obtained. A liberal cutoff Ct value is then obtained using the equation:

Liberal cutoff Ct value=normalized ΔCt value for HMGA2−log 2(18.86/9.82)

In an exemplary embodiment, a liberal cutoff Ct value of 11.6 is obtained using a ΔCt value of 12.1 suggesting that patients with a Ct value of 11.16 or less are classified as HMGA2 high and suitable for treatment with anti-PD-L1/TGFβ Trap.

Similarly, a conservative cutoff Ct value may be obtained using the equation:

Conservative cutoff Ct value=normalized ΔCt value for HMGA2−log 2(177.75/9.82)

In an exemplary embodiment, a conservative cutoff Ct value of 7.92 is obtained using a ΔCt value of 12.1 suggesting that patients with a Ct value of 7.92 or less are classified as HMGA2 high and suitable for treatment with anti-PD-L1/TGFβ Trap.

HMGA2 Expression Cutoff using ddPCR Ratio Values (Fold-Change Derivation Method): In parallel, ddPCR experiment is performed on 58 out of 103 tumor samples using methods described in Example 4 to obtain expression values for HMGA2 and a housekeeping gene, beta-actin. In certain embodiments, quantitation of HMGA2 expression relative to beta-actin by ddPCR is performed to establish a cutoff between high and low HMGA2 expression. In an exemplary embodiment, a median ddPCR ratio of 0.054 is obtained using the equation below:

ddPCR ratio=(HMGA2 copy number/beta-actin copy number)×10000

Using the median ddPCR ratio, a liberal cutoff value is obtained using the equation:

Liberal cutoff value=median ddPCR ratio for HMGA2×(18.86/9.82)

In an exemplary embodiment, a liberal cutoff value of 0.104 is obtained using a median ddPCR ratio of 0.054 suggesting that patients with a ddPCR ratio of 0.104 or greater are classified as HMGA2 high and suitable for treatment with anti-PD-L1/TGFβ Trap. Similarly, a conservative cutoff value may be obtained using the equation:

Conservative cutoff value=median ddPCR for HMGA2×(177.75/9.82)

In an exemplary embodiment, a conservative cutoff value of 0.976 is obtained using a median ddPCR value of 0.054 suggesting that patients with a ddPCR ratio of 0.976 or greater are classified as HMGA2 high and suitable for treatment with anti-PD-L1/TGFβ Trap.

HMGA2 Expression Data from RNA-seq (Percentile Derivation Method): In an exemplary population cohort of TNBC patients treated with anti-PD-L1/TGFβ Trap protein, formalin-fixed, paraffin embedded (FFPE) tumor samples (n=118) are used to extract RNA and assess the quality of RNA as described in Example 4. Analysis of RNA-seq (as described in Example 1) performed on samples that passed the quality control (n=103) shows that lowest HMGA2 expression among responders ranked 22^{nd} out of 28 samples corresponding to the 78.6^{th} percentile. The HMGA2 expression second lowest among responders ranked 26^{th} out of 28 samples corresponding to the 92.9^{th} percentile.

HMGA2 Expression Cutoff using Ct Values (Percentile Derivation Method): In certain embodiments, a qPCR experiment is performed to obtain relative quantitation of HMGA2 expression to establish a cutoff between high and low HMGA2 expression. In an exemplary embodiment, the relative qPCR expression at the 78.6^{th} percentile is a ΔCt value of 8.7 (liberal cutoff), suggesting that patients with ΔCt value of 8.7 or less would be classified as HMGA2 high and suitable for treatment with anti-PD-L1/TGFβ Trap. In another exemplary embodiment, the relative qPCR expression at the 92.9^{th} percentile is a ΔCt value of 6.9 (conservative cutoff), suggesting that patients with ΔCt value of 6.9 or less would be classified as HMGA2 high and suitable for treatment with anti-PD-L1/TGFβ Trap.

HMGA2 Expression Cutoff using ddPCR Ratio Values (Percentile Derivation Method): In certain embodiments, HMGA2 expression can be quantified by ddPCR to establish a cutoff between high and low HMGA2 expression. In an exemplary embodiment, the relative ddPCR expression at the 78.6^{th} percentile is a ddPCR ratio value of 0.467 (liberal cutoff), suggesting that patients with HMGA2 relative expression of 0.467 or more would be classified as HMGA2 high and suitable for treatment with anti-PD-L1/TGFβ Trap. In another exemplary embodiment, the relative ddPCR expression at the 92.9^{th} percentile is 1.375 (conservative cutoff), suggesting that patients with HMGA2 relative expression of 1.375 or more would be classified as HMGA2 high and suitable for treatment with anti-PD-L1/TGFβ Trap. Table 7 lists exemplary expression cutoff values obtained by qPCR and ddPCR, and analyzed by fold-change derivation method and percentile derivation method. The cutoff values depend on the power of the analytical method (for example population cohort size), and can vary depending on the sample size and the differences between population characteristics (for example, age, gender, ethnic origin, smoking habits, dietary habits, body-mass index (BMI), recreational drug use, medical drug use, and/or exercise habits).

TABLE 7
Summary of HMGA2 expression cutoff values and number of samples
predicted to respond to a treatment with anti-PD-L1/TGFβ Trap as calculated.

| | | | |
|---|---|---|---|
| | | ddPCR | ddPCR |
| | qPCR | Liberal | Conservative |
| qPCR Liberal | Conservative | cutoff | cutoff |
| cutoff (ΔCt | cutoff (ΔCt | (ddPCR | (ddPCR |
| value) | value) | ratio) | ratio) |

Fold-Change Derivation Method

| | | | | |
|---|---|---|---|---|
| HMGA2 expression cutoff | 11.16 | 7.92 | 0.104 | 0.976 |
| Number of samples with high | 43/103 | 16/103 | 25/58 | 4/58 |
| HMGA2 expression based on the | (41.7%) | (15.5%) | (43.1%) | (6.9%) |
| cutoff values | | | | |

Percentile Derivation Method

| | | | | |
|---|---|---|---|---|
| HMGA2 expression cutoff | 8.7 | 6.9 | 0.467 | 1.375 |
| Number of samples with high | 22/103 | 7/103 | 12/58 | 4/58 |
| HMGA2 expression based on the | (21.4%) | (6.8%) | (20.7%) | (6.9%) |
| cutoff values | | | | |

### Example 5: Treatment of TNBC Patients Refractory or Resistant to Prior Treatment

Objective: Patients with metastatic and refractory (3L+) triple negative breast cancer (TNBC) were selected for treatment with 1200 mg of anti-PD-L1/TGFβ Trap therapy and safety and efficacy was assessed.

Study Design and Results: A total of 33 patients were treated with anti-PD-L1/TGFβ Trap at a dose of 1200 mg every 2 weeks until confirmed progressive disease, unacceptable toxicity, or trial withdrawal. The safety summary of the anti-PD-L1/TGFβ Trap expansion cohort in triple negative breast cancer patients is listed as below. Patients in this cohort received 1,200 mg of anti-PD-L1/TGFβ Trap every 2 weeks.

33 patients enrolled in this cohort had median follow-up of 18.0 weeks (range: 4.0-31.7 weeks) and received a mean of 3.8 doses (range: 1.0-12.0 doses). Two patients (6.1%) were ongoing. 31 patients discontinued the trial due to progressive disease (n=26, 78.8%), death (n=1, 3.0%, due to disease progression), adverse event (n=2, 6.1%, transaminitis, hemolysis), protocol non-compliance (n=1, 3.0%, patient unable to keep appointments) and withdrew consent (n=1, 3.0%, clinically progressing and admitted to hospice).

Table 8 lists Treatment emergent adverse events (TEAEs) irrespective of relationship to anti-PD-L1/TGFβ Trap occurring in 3 or more patients as well as all AEs that are grade 3 or higher. The most common TEAEs included dyspnea (n=10, 30.3%), anemia (n=9, 27.3%), diarrhea (n=8, 24.2%), asthenia (n=8, 24.2%), pyrexia (n=8, 24.2%), decreased appetite (8, 24.2%) and headache (n=8, 24.2%). There were 5 patients (15.2%) with 7 grade 3+ events assessed as related to anti-PD-L1/TGFβ Trap by investigator. These include hemolysis (grade 5), thrombocytopenia (grade 5), dyspnea (grade 5), anemia (grade 3, 3 events) and increased transaminases (grade 3). The 3 G5 events assessed by study investigator as related to anti-PD-L1/TGFβ Trap occurred in one patient who had extensive disease at trial entry, and was concurrently found to have multiple pulmonary emboli, progressive disease and expanding pleural effusion after 3 doses. No auto-antibodies mediating hemolysis or thrombocytopenia were identified on work-up. Skin lesions including keratoacanthoma and cutaneous squamous cell carcinoma (similar to those identified with other TGF-β-inhibiting agents), occurred in approximately 3-5% of all dosed patients on trial and were well-managed by surgical excision. However, none of these cutaneous lesions occurred in this cohort. In summary, anti-PD-L1/TGFβ Trap was well-tolerated and the safety profile was consistent with expectations in this heavily-pretreated, advanced, triple negative breast cancer cohort.

TABLE 8
Treatment Emergent Adverse Events (TEAEs) in Triple negative breast
cancer patients with anti-PD-L1/TGFβ Trap. Preferred terms are listed for AE
occurring in three or more patients, and all grade 3+ events. Events are listed
irrespective of relationship to anti-PD-L1/TGFβ Trap.

| Primary System Organ Class | Any Grade | Grade ≥ 3 | Grade ≥ 4 | Grade 5 |
|---|---|---|---|---|
| Preferred Term | n (%) | n (%) | n (%) | n (%) |
| Subjects with at least 1 event | 33 (100%) | 19 (57.6) | 11 (33.3) | 8 (24.2) |
| Dyspnea | 10 (30.3) | 3 (9.1) | 1 (3.0) | 1 (3.0) |
| Anemia | 9 (27.3) | 5 (15.2) | 0 | 0 |
| Diarrhea | 8 (24.2) | 0 | 0 | 0 |
| Asthcnia | 8 (24.2) | 2 (6.1) | 2 (6.1) | 0 |
| Pyrexia | 8 (24.2) | 1 (3.0) | 0 | 0 |
| Decreased appetite | 8 (24.2) | 0 | 0 | 0 |
| Headache | 8 (24.2) | 0 | 0 | 0 |
| Abdominal pain | 5 (15.2) | 0 | 0 | 0 |
| Constipation | 5 (15.2) | 0 | 0 | 0 |
| Nausea | 5 (15.2) | 0 | 0 | 0 |
| Vomiting | 5 (15.2) | 0 | 0 | 0 |
| Disease progression | 5 (15.2) | 4 (12.1) | 4 (12.1) | 4 (12.1) |
| Increased AST | 5 (15.2) | 2 (6.1) | 0 | 0 |
| Pain in extremity | 5 (15.2) | 1 (3.0) | 0 | 0 |
| Epistaxis | 4 (12.1) | 0 | 0 | 0 |
| Cough | 5 (15.2) | 0 | 0 | 0 |
| Upper respiratory tract infection | 4 (12.1) | 0 | 0 | 0 |
| Hypokalemia | 4 (12.1) | 1 (3.1) | 0 | 0 |
| Anxiety | 4 (12.1) | 0 | 0 | 0 |
| Pleural effusion | 4 (12.1) | 2 (6.1) | 0 | 0 |
| Pruritus | 4 (12.1) | 0 | 0 | 0 |
| Tachycardia | 3 (9.1) | 0 | 0 | 0 |
| General physical health | 3 (9.1) | 3 (9.1) | 1 (3.0) | 1 (3.0) |
| deterioration | | | | |
| Fatigue | 3 (9.1) | 1 (3.0) | 0 | 0 |
| Peripheral edema | 3 (9.1) | 0 | 0 | 0 |
| Dry skin | 3 (9.1) | 0 | 0 | 0 |
| Rash | 3 (9.1) | 0 | 0 | 0 |
| Increased ALT | 2 (6.1) | 1 (3.0) | 0 | 0 |
| Increased ALP | 2 (6.1) | 2 (6.1) | 0 | 0 |
| Increased GGT | 2 (6.1) | 2 (6.1) | 0 | 0 |
| Increased blood bilirubin | 2 (6.1) | 1 (3.0) | 0 | 0 |
| Hypoalbuminemia | 2 (6.1) | 1 (3.0) | 0 | 0 |
| Arthralgia | 2 (6.1) | 1 (3.0) | 0 | 0 |
| Hemolysis | 1 (3.0) | 1 (3.0) | 1 (3.0) | 1 (3.0) |
| Leukocytosis | 1 (3.0) | 1 (3.0) | 0 | 0 |
| Thrombocytopenia | 1 (3.0) | 1 (3.0) | 1 (3.0) | 1 (3.0) |
| Thrombotic microangiopathy | 1 (3.0) | 1 (3.0) | 1 (3.0) | 1 (3.0) |
| Cardiac tamponade | 1 (3.0) | 1 (3.0) | 1 (3.0) | 0 |
| Multiple organ dysfunction | 1 (3.0) | 1 (3.0) | 1 (3.0) | 1 (3.0) |
| Mastitis | 1 (3.0) | 1 (3.0) | 0 | 0 |
| Contusion | 1 (3.0) | 1 (3.0) | 0 | 0 |
| Spinal compression fracture | 1 (3.0) | 1 (3.0) | 0 | 0 |
| Increased amylase | 1 (3.0) | 1 (3.0) | 0 | 0 |
| Decreased hemoglobin | 1 (3.0) | 1 (3.0) | 0 | 0 |
| Decreased lymphocyte count | 1 (3.0) | 1 (3.0) | 1 (3.0) | 0 |
| Increased transaminases | 1 (3.0) | 1 (3.0) | 0 | 0 |
| Hypomagnesemia | 1 (3.0) | 1 (3.0) | 0 | 0 |
| Hyponatremia | 1 (3.0) | 1 (3.0) | 0 | 0 |
| Musculoskeletal pain | 1 (3.0) | 1 (3.0) | 0 | 0 |
| Metastases to central nervous system | 1 (3.0) | 1 (3.0) | 1 (3.0) | 0 |
| Tumor pain | 1 (3.0) | 1 (3.0) | 0 | 0 |
| Encephalopathy | 1 (3.0) | 1 (3.0) | 0 | 0 |
| Pulmonary embolism | 1 (3.0) | 1 (3.0) | 0 | 0 |
| Skin lesion | 1 (3.0) | 1 (3.0) | 0 | 0 |
| Jugular vein thrombosis | 1 (3.0) | 1 (3.0) | 0 | 0 |
| Lymphoedema | 1 (3.0) | 1 (3.0) | 0 | 0 |
| Superior vena cava syndrome | 1 (3.0) | 1 (3.0) | 0 | 0 |

As described in the narrative, 1 patient had 3 G5 TEAEs assessed as related to anti-PD-L1/TGFβ Trap (dyspnea, hemolysis, thrombocytopenia) by the study investigator. The additional 7 patients with G5 events were not ascribed as related to anti-PD-L1/TGFβ Trap. Per protocol, the TEAE “Progressive Disease,” was documented when a patient had died due to progressive disease within 28 days of most recent drug administration, or if disease progression was assessed by the investigator to have occurred more rapidly than expected.

In one exemplary embodiment, anti-PD-L1/TGFβ Trap is administered as a BW-independent dose of 1800 mg to cancer patients with TNBC once every three weeks. The administration is performed intravenously for about an hour (−10 minutes/+20 minutes, e.g., 50 minutes to 80 minutes). In one exemplary embodiment, anti-PD-L1/TGFβ Trap is administered as a BW-independent dose of 2100 mg to cancer patients with TNBC once every three weeks. The administration is performed intravenously for about an hour (−10 minutes/+20 minutes, e.g., 50 minutes to 80 minutes). In one exemplary embodiment, anti-PD-L1/TGFβ Trap is administered as BW-independent dose of 2400 mg to cancer patients with TNBC once every three weeks. The administration is performed intravenously for about an hour (−10 minutes/+20 minutes, e.g., 50 minutes to 80 minutes). In one exemplary embodiment, anti-PD-L1/TGFβ Trap is administered as BW-independent dose of 2600 mg, 2800 mg, or 3000 mg to cancer patients with TNBC once every three weeks. The administration is performed intravenously for about an hour (−10 minutes/+20 minutes, e.g., 50 minutes to 80 minutes). In one or more exemplary embodiments, in order to mitigate potential infusion-related reactions, premedication with an antihistamine and with paracetamol (acetaminophen) (for example, 25-50 mg diphenhydramine and 500-650 mg paracetamol [acetaminophen] IV or oral equivalent) approximately 30 to 60 minutes prior to each anti-PD-L1/TGFβ Trap dose is administered for the first 2 infusions. If Grade ≥2 infusion reactions are observed during the first two infusions, premedication is not stopped. Steroids as premedication are not permitted.

The following describes the inclusion criteria for patients used in this example. Patients:
are ≥18 years, inclusive at the time of informed consent
have histologically confirmed diagnosis of TNBC
have measurable disease based on RECIST 1.1 (see Eisenhauer et al., EJC. 2009; 45:228-247)
have not received prior systemic therapy treatment, or any antibody or drug targeting T-cell coregulatory proteins (immune checkpoints) such as anti-PDL1, or anti-CTLA-4 antibody, since diagnosis of metastatic and refractory (3L+) TNBC (completion of treatment with cytotoxic chemotherapy, biological therapy, and/or radiation as part of neoadjuvant/adjuvant therapy is allowed as long as therapy was completed at least 6 months prior to the diagnosis of metastatic disease)
have a life expectancy of at least 12 weeks (based on physician's assessment of the prognosis of the patient after diagnosis)
have available tumor material (<6 months old) adequate for biomarker analysis
have Eastern Cooperative Oncology Group Performance Status (ECOG PS) of 0 to 1
have adequate hematological function considered as absolute neutrophil count (ANC)≥1.5×10⁹/L, platelet count ≥100×10⁹/L, and Hgb≥9 g/dL
have adequate hepatic function considered as a total bilirubin level ≤1.5× upper limit of normal (ULN), an aspartate aminotransferase (AST) level ≤3.0×ULN, an alanine aminotransferase (ALT) level ≤3.0×ULN and alkaline phosphatase ≤2.5 ULN. For participants with liver involvement in their tumor, aspartate aminotransferase (AST)≤5.0×ULN, alanine aminotransferase (ALT)≤5.0×ULN, and bilirubin ≤3.0×ULN is acceptable
have adequate renal function considered as creatinine ≤1.5×ULN or a calculated creatinine clearance >30 mL/min; and
have adequate coagulation function considered as international normalized ratio (INR) or prothrombin time (PT)≤1.5×ULN unless the participant is receiving anticoagulant therapy, and activated partial thromboplastin time (aPTT)≤1.5×ULN unless the participant is receiving anticoagulant therapy.

### Example 6: Treatment of TNBC Patients with High HMGA2 Expression

Objective: The purpose of this study is to determine the best overall response (BOR) of anti-PD-L1/TGFβ Trap treatment in patients with advanced triple negative breast cancer (TNBC) who have tumors with high HMGA2 expression and disease progression on or after first line systemic chemotherapy.

Study Design: This is a phase II single arm biomarker-driven trial to evaluate clinical efficacy of anti-PD-L1/TGFβ Trap in patients with advanced triple negative breast cancer (TNBC) with high expression of HMGA2.

In one exemplary embodiment, the tumors from TNBC patients are screened for high HMGA2 expression, considered as an HMGA2 expression level that is at least 2.27 times higher than the population mean among TNBC patients. Tumor material is required for all participants to ascertain HMGA2 status by centralized RT-PCR and may include fresh biopsy or archival material.

Approximately 29 patients meeting the predetermined cutoff for high HMGA2 expression are enrolled in the study. Patients are treated with anti-PD-L1/TGFβ Trap protein at 1200 mg per infusion once every 14 days (+/−3 days). Treatment is continued until confirmed disease progression, unacceptable toxicity, sustained confirmed complete response, or trial withdrawal for a period of up to two years. Optionally, longer treatment and treatment past confirmed disease progression is possible after discussion with the study medical monitory and if it is determined that the patient may benefit from continued treatment.

In order to mitigate potential infusion-related reactions, premedication with an antihistamine and with acetaminophen is optionally be administered prior to the first two doses of anti-PD-L1/TGFβ Trap. Patients who have been premedicated with steroids are not excluded from the study.

Efficacy Assessments: Response to anti-PD-L1/TGFβ Trap treatment is assessed by CT imaging every 6-8 weeks+/−7 days according to RECIST 1.1 criteria. Scans performed at baseline are repeated at subsequent visits. In general, lesions detected at baseline are followed using the same imaging methodology and preferably the same imaging equipment at subsequent tumor evaluation visits. Overall response rate (ORR), progression-free survival (PFS) and duration of response (DOR) is calculated and compared with historical control.

Treatment is continued until confirmed progressive disease (PD) per Response Evaluation Criteria in Solid Tumors version 1.1 (RECIST 1.1), unacceptable toxicity, or for up to 24 months. Patients who experience stable disease (SD), partial response (PR), or complete response (CR) will continue treatment until the end of 24 months, although additional treatment is be possible. Treatment past confirmed disease progression will be possible after discussion with the study medical monitory if it is determined that the patient may benefit from continued treatment.

Throughout treatment, safety of anti-PD-L1/TGFβ Trap treatment is assessed in patients with advanced, treatment-experienced, triple negative breast cancer (TNBC) with high HMGA2 through the recording, reporting and analysis of baseline medical conditions, adverse events (AEs), physical examination findings, including vital signs, ECOG performance status, and laboratory tests.

Results: Objective tumor response is evaluated by the overall response rate (ORR), defined as the number of participants having reached a best overall response (BOR) of complete response (CR) or partial response (PR) divided by the number of participants in the analysis population. Progression-free survival is defined as the time from randomization to the date of the first documentation of objective progression of disease (PD) as assessed according to RECIST 1.1 or death due to any cause, whichever occurs first. It is contemplated that treatment of with anti-PD-L1/TGFβ Trap results in improved clinical response for high HMGA2-expressing TNBC patients. Treated patients exhibit disease response (e.g., partial response, complete response, stable disease) and/or improved survival (e.g., progression-free survival and/or overall survival). It is contemplated that treatment with anti-PD-L1/TGFβ Trap results in superior survival of high HMGA2-expressing TNBC patients compared to systemic chemotherapy.

In another exemplary embodiment, TNBC patients are screened for high MECOM expression, considered as MECOM expression level that is 1.73 times higher than the population mean among TNBC patients. The same study is then conducted with 30 patients meeting the predetermined cutoff for high MECOM expression.

In summary, HMGA2 are found to be reliable new biomarkers for determining improved response to treatment with anti-PD-L1/TGFβ Trap in TNBC patients.

### NUMBERED EMBODIMENTS

Embodiments disclosed herein include embodiments P1 to P92 as provided in the numbered embodiments of the disclosure.

Embodiment P1: A method of treating or managing triple negative breast cancer (TNBC) in a patient, the method comprising administering an anti-TGFβ agent to a patient who has been determined to have an increased level of high mobility group AT-hook 2 (HMGA2) expression relative to a known control level, and thereby treating TNBC in the patient.

Embodiment P2: A method of achieving at least a partial response in treating or increasing survival of a triple negative breast cancer (TNBC) patient, the method comprising administering an anti-TGFβ agent to a patient who has been determined to have an increased level of high mobility group AT-hook 2 (HMGA2) expression relative to a known control level, and thereby achieving at least a partial response in treating TNBC in the patient.

Embodiment P3: A method of identifying a patient suitable for treating or managing triple negative breast cancer (TNBC) in the patient with an anti-TGFβ agent, the method comprising determining the level of high mobility group AT-hook 2 (HMGA2) in the patient, wherein an increased level of HMGA2 expression in the patient, relative to a known control level, identifies the patient as suitable for treating TNBC with said anti-TGFβ agent.

Embodiment P4: The method of any one of embodiments P1 to P3, wherein the HMGA2 level of the patient is determined by analyzing a tissue sample from the patient.

Embodiment P5: The method of embodiment P4, wherein the tissue sample is a biopsy sample, blood, serum, or plasma sample.

Embodiment P6: The method of embodiment P4 or P5, wherein the level of HMGA2 is determined by immunochemistry or by RNA expression analysis.

Embodiment P7: The method of any one of embodiments P1 to P6, wherein the anti-TGFβ agent is an anti-PD-L1/TGFβ Trap protein comprising a first polypeptide comprising: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), and a second polypeptide comprising at least a variable region of a light chain of an antibody that binds PD-L1; wherein the heavy chain of the first polypeptide and the light chain of the second polypeptide, when combined, form an antigen binding site that binds PD-L1.

Embodiment P8: The method of embodiment P7, wherein the patient is administered at least 1200 mg of the anti-TGFβ agent.

Embodiment P9: The method of embodiment P7, wherein the patient is administered at least 1800 mg of the anti-PD-L1/TGFβ Trap protein.

Embodiment P10: The method of embodiment P7, wherein the patient is administered 1800 mg to 3000 mg of the anti-PD-L1/TGFβ Trap protein.

Embodiment P11: The method of embodiment P7, wherein the patient is administered 1800 mg to 2100 mg of the anti-PD-L1/TGFβ Trap protein.

Embodiment P12: The method of embodiment P7, wherein the patient is administered 1200 mg of the anti-PD-L1/TGFβ Trap protein.

Embodiment P13: The method of embodiment P12, wherein the patient is administered 1200 mg of the anti-PD-L1/TGFβ Trap protein, once every three weeks.

Embodiment P14: The method of embodiment P10, wherein the patient is administered 2400 mg of the anti-PD-L1/TGFβ Trap protein.

Embodiment P15: The method of embodiment P14, wherein the patient is administered 2400 mg of the anti-PD-L1/TGFβ Trap protein, once every three weeks.

Embodiment P16: The method of embodiment P10, wherein the patient is administered 2100 mg or 3000 mg of the anti-PD-L1/TGFβ Trap protein, once every three weeks.

Embodiment P17: The method of any one of embodiments P1 to P16, wherein the increased HMGA2 expression has been determined via quantification of HMGA2 mRNA expression.

Embodiment P18: The method of embodiment P17, wherein the quantification of HMGA2 mRNA expression is via PCR.

Embodiment P19: The method of any one of embodiments P1 to P18, wherein the increased HMGA2 expression is at least 2.27-fold more than a known population mean HMGA2 expression among TNBC patients.

Embodiment P20: The method of any one of embodiments P1 to P19, wherein the increased HMGA2 expression is at least 5-fold more than a known population mean HMGA2 expression among TNBC patients.

Embodiment P21: The method of any one of embodiments P1 to P18, wherein the increased HMGA2 expression is at least 200% higher, at least 300% higher, at least 400% higher, at least 500% higher, at least 600% higher, at least 700% higher, at least 800% higher, at least 900% higher, at least 1000% higher, or more than the normal level of HMGA2 expression.

Embodiment P22: The method of any one of embodiments P1 to P18, wherein the increased HMGA2 expression in the patient is at least 19- to 35-fold more than the HMGA2 expression in a patient who is non-responsive to a treatment with the anti-TGFβ agent.

Embodiment P23: The method of any one of embodiments P1 to P16, wherein the increased HMGA2 expression has been determined by HMGA2 protein expression level.

Embodiment P24: The method of embodiment P23, wherein the increased HMGA2 protein expression level has been determined via immunohistochemistry.

Embodiment P25: The method of embodiment P24, wherein more than 1% tumor cells expressing HMGA2 protein in a tissue sample obtained from the TNBC patient determined the increased HMGA2 protein expression level.

Embodiment P26: A method of treating or managing triple negative breast cancer (TNBC) in a patient, the method comprising administering an anti-TGFβ agent to a patient who has been determined to have an increased level of MECOM expression relative to a known control level, and thereby treating TNBC in the patient.

Embodiment P27: A method of achieving at least a partial response in treating or increasing survival of a triple negative breast cancer (TNBC) patient, the method comprising administering an anti-TGFβ agent to a patient who has been determined to have an increased level of MECOM expression relative to a known control level, and thereby achieving at least a partial response in treating TNBC in the patient.

Embodiment P28: A method of identifying a patient suitable for treating or managing triple negative breast cancer (TNBC) of the patient with an anti-TGFβ agent, the method comprising determining the level of MECOM in the patient, wherein an increased level of MECOM expression in the patient, relative to a known control level, identifies the patient as suitable for treating TNBC with the anti-TGFβ agent.

Embodiment P29: The method of any one of embodiments P26 to P28, wherein the MECOM level of the patient is determined by analyzing a sample from the patient.

Embodiment P30: The method of embodiment P29, wherein the sample is a biopsy sample, blood, serum, or plasma sample.

Embodiment P31: The method of embodiment P29 or P30, wherein the level of MECOM is determined by immunochemistry or by RNA expression analysis.

Embodiment P32: The method of any one of embodiments P26 to P31, wherein the anti-TGFβ agent is an anti-PD-L1/TGFβ Trap protein comprising a first polypeptide comprising: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), and a second polypeptide comprising at least a variable region of a light chain of an antibody that binds PD-L1; wherein the heavy chain of the first polypeptide and the light chain of the second polypeptide, when combined, form an antigen binding site that binds PD-L1.

Embodiment P33: The method of embodiment P32, wherein the patient is administered at least 1200 mg of the anti-PD-L1/TGFβ Trap protein.

Embodiment P34: The method of embodiment P32 or P33, wherein the patient is administered at least 1800 mg of the anti-PD-L1/TGFβ Trap protein.

Embodiment P35: The method of embodiment P34, wherein the patient is administered 1800 mg to 3000 mg of the anti-PD-L1/TGFβ Trap protein.

Embodiment P36: The method of embodiment P35, wherein the patient is administered 1800 mg to 2100 mg of the anti-PD-L1/TGFβ Trap protein.

Embodiment P37: The method of embodiment P36, wherein the patient is administered 1200 mg of the protein.

Embodiment P38: The method of embodiment P37, wherein the patient is administered 1200 mg of the anti-PD-L1/TGFβ Trap protein, once every two weeks.

Embodiment P39: The method of embodiment P35, wherein the patient is administered 2400 mg of the anti-PD-L1/TGFβ Trap protein.

Embodiment P40: The method of embodiment P39, wherein the patient is administered 2400 mg of the anti-PD-L1/TGFβ Trap protein, once every three weeks.

Embodiment P41: The method of embodiment P35, wherein the patient is administered 3000 mg of the anti-PD-L1/TGFβ Trap protein, once every three weeks.

Embodiment P42: The method of any one of embodiments P26 to P41, wherein the increased MECOM expression has been determined via quantification of MECOM mRNA expression.

Embodiment P43: The method of embodiment P42, wherein the quantification of MECOM mRNA expression is via PCR.

Embodiment P44: The method of any one of embodiments P26 to P43, wherein the increased MECOM expression is at least 1.5-fold more than a known population average level of MECOM expression among TNBC patients.

Embodiment P45: The method of any one of embodiments P26 to P43, wherein the increased MECOM expression is at least 2.5-fold more than the known population average level of MECOM expression among TNBC patients.

Embodiment P46: The method of any one of embodiments P26 to P43, wherein the increased MECOM expression is at least 100%, at least 200% higher, at least 300% higher, at least 400% higher, at least 500% higher, at least 600% higher, at least 700% higher, at least 800% higher, at least 900% higher, at least 1000% higher, or more than the normal level of MECOM expression.

Embodiment P47: The method of any one of embodiments P26 to P43, wherein the increased MECOM expression in the patient is at least 19-fold more than the MECOM expression in a patient who is non-responsive to a treatment with the anti-TGFβ agent.

Embodiment P48: The method of any one of embodiments P26 to P41, wherein the increased MECOM expression has been determined via quantification of the MECOM protein.

Embodiment P49: The method of embodiment P48, wherein the increased MECOM protein level has been determined via immunohistochemistry.

Embodiment P50: The method of embodiment P49, wherein more than 1% tumor cells expressing MECOM protein in a tissue sample obtained from the TNBC patient determined the increased MECOM protein expression level.

Embodiment P51: An anti-TGFβ agent for use in a method of treating or managing triple negative breast cancer (TNBC) in a patient, the method comprising administering an anti-TGFβ agent to a patient who has been determined to have an increased level of high mobility group AT-hook 2 (HMGA2) expression relative to a known control level, and thereby treating TNBC in the patient.

Embodiment P52: An anti-TGFβ agent for use in a method of achieving at least a partial response in treating or increasing survival of a triple negative breast cancer (TNBC) patient, the method comprising administering an anti-TGFβ agent to a patient who has been determined to have an increased level of high mobility group AT-hook 2 (HMGA2) expression relative to a known control level, and thereby achieving at least a partial response in treating TNBC in the patient.

Embodiment P53: An anti-TGFβ agent for use in a method of identifying a patient suitable for treating or managing triple negative breast cancer (TNBC) in the patient with an anti-TGFβ agent, the method comprising determining the level of high mobility group AT-hook 2 (HMGA2) in the patient, wherein an increased level of HMGA2 expression in the patient, relative to a known control level, identifies the patient as suitable for treating TNBC with said anti-TGFβ agent.

Embodiment P54: The anti-TGFβ agent for use of any one of embodiments P51 to P53, wherein the HMGA2 level of the patient is determined by analyzing a tissue sample from the patient.

Embodiment P55: The anti-TGFβ agent for use of embodiment P54, wherein the tissue sample is a biopsy sample, blood, scrum, or plasma sample.

Embodiment P56: The anti-TGFβ agent for use of embodiment P54 or P55, wherein the level of HMGA2 is determined by immunochemistry or by RNA expression analysis.

Embodiment P57: The anti-TGFβ agent for use of any one of embodiments P51 to P56, wherein the increased HMGA2 expression has been determined via quantification of HMGA2 mRNA expression.

Embodiment P58: The anti-TGFβ agent for use of embodiment P57, wherein the quantification of HMGA2 mRNA expression is via PCR.

Embodiment P59: The anti-TGFβ agent for use of any one of embodiments P51 to P58, wherein the increased HMGA2 expression is at least 2.27-fold more than a known population mean HMGA2 expression among TNBC patients.

Embodiment P60: The anti-TGFβ agent for use of any one of embodiments P51 to P59, wherein the increased HMGA2 expression is at least 5-fold more than a known population mean HMGA2 expression among TNBC patients.

Embodiment P61: The anti-TGFβ agent for use of any one of embodiments P51 to P58, wherein the increased HMGA2 expression is at least 200% higher, at least 300% higher, at least 400% higher, at least 500% higher, at least 600% higher, at least 700% higher, at least 800% higher, at least 900% higher, at least 1000% higher, or more than the normal level of HMGA2 expression.

Embodiment P62: The anti-TGFβ agent for use of any one of embodiments P51 to P58, wherein the increased HMGA2 expression in the patient is at least 19- to 35-fold more than the HMGA2 expression in a patient who is non-responsive to a treatment with the anti-TGFβ agent.

Embodiment P63: The anti-TGFβ agent for use of any one of embodiments P51 to P56, wherein the increased HMGA2 expression has been determined by HMGA2 protein expression level.

Embodiment P64: The anti-TGFβ agent for use of embodiment P63, wherein the increased HMGA2 protein expression level has been determined via immunohistochemistry.

Embodiment P65: The anti-TGFβ agent for use of embodiment P64, wherein more than 1% tumor cells expressing HMGA2 protein in a tissue sample obtained from the TNBC patient determined the increased HMGA2 protein expression level.

Embodiment P66: An anti-TGFβ agent for use in a method of treating or managing triple negative breast cancer (TNBC) in a patient, the method comprising administering an anti-TGFβ agent to a patient who has been determined to have an increased level of MECOM expression relative to a known control level, and thereby treating TNBC in the patient.

Embodiment P67: An anti-TGFβ agent for use in a method of achieving at least a partial response in treating or increasing survival of a triple negative breast cancer (TNBC) patient, the method comprising administering an anti-TGFβ agent to a patient who has been determined to have an increased level of MECOM expression relative to a known control level, and thereby achieving at least a partial response in treating TNBC in the patient.

Embodiment P68: An anti-TGFβ agent for use in a method of identifying a patient suitable for treating or managing triple negative breast cancer (TNBC) of the patient with an anti-TGFβ agent, the method comprising determining the level of MECOM in the patient, wherein an increased level of MECOM expression in the patient, relative to a known control level, identifies the patient as suitable for treating TNBC with the anti-TGFβ agent.

Embodiment P69: The anti-TGFβ agent for use of any one of embodiments P66 to P68, wherein the MECOM level of the patient is determined by analyzing a sample from the patient.

Embodiment P70: The anti-TGFβ agent for use of embodiment P69, wherein the sample is a biopsy sample, blood, serum, or plasma sample.

Embodiment P71: The anti-TGFβ agent for use of embodiment P69 or P70, wherein the level of MECOM is determined by immunochemistry or by RNA expression analysis.

Embodiment P72: The anti-TGFβ agent for use of any one of embodiments P66 to P71, wherein the increased MECOM expression has been determined via quantification of MECOM mRNA expression.

Embodiment P73: The anti-TGFβ agent for use of embodiment 72, wherein the quantification of MECOM mRNA expression is via PCR.

Embodiment P74: The anti-TGFβ agent for use of any one of embodiments P66 to P73, wherein the increased MECOM expression is at least 1.5-fold more than a known population average level of MECOM expression among TNBC patients.

Embodiment P75: The anti-TGFβ agent for use of any one of embodiments P66 to P73, wherein the increased MECOM expression is at least 2.5-fold more than the known population average level of MECOM expression among TNBC patients.

Embodiment P76: The anti-TGFβ agent for use of any one of embodiments P66 to P73, wherein the increased MECOM expression is at least 100%, at least 200% higher, at least 300% higher, at least 400% higher, at least 500% higher, at least 600% higher, at least 700% higher, at least 800% higher, at least 900% higher, at least 1000% higher, or more than the normal level of MECOM expression.

Embodiment P77: The anti-TGFβ agent for use of any one of embodiments P66 to P73, wherein the increased MECOM expression in the patient is at least 19-fold more than the MECOM expression in a patient who is non-responsive to a treatment with the anti-TGFβ agent.

Embodiment P78: The anti-TGFβ agent for use of any one of embodiments P66 to P71, wherein the increased MECOM expression has been determined via quantification of the MECOM protein.

Embodiment P79: The anti-TGFβ agent for use of embodiment P78, wherein the increased MECOM protein level has been determined via immunohistochemistry.

Embodiment P80: The anti-TGFβ agent for use of embodiment P79, wherein more than 1% tumor cells expressing MECOM protein in a tissue sample obtained from the TNBC patient determined the increased MECOM protein expression level.

Embodiment P81: The anti-TGFβ agent for use of any one of embodiments P51 to P80, wherein the anti-TGFβ agent is an anti-PD-L1/TGFβ Trap protein comprising a first polypeptide comprising: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), and a second polypeptide comprising at least a variable region of a light chain of an antibody that binds PD-L1; wherein the heavy chain of the first polypeptide and the light chain of the second polypeptide, when combined, form an antigen binding site that binds PD-L1.

Embodiment P82: The anti-TGFβ agent for use of any one of embodiments P51 to P81, wherein the dose is 1200 mg to 3000 mg.

Embodiment P83: The anti-TGFβ agent for use of embodiment P82, wherein the dose is 1200 mg.

Embodiment P84: The anti-TGFβ agent for use of embodiment P83, wherein the dose is 1200 mg, administered once every two weeks.

Embodiment P85: The anti-TGFβ agent for use of any one of embodiments P51 to P82, wherein the dose is 2100 mg to 2400 mg.

Embodiment P86: The anti-TGFβ agent for use of embodiment P85, wherein the protein is administered once every three weeks.

Embodiment P87: The anti-TGFβ agent for use of embodiment P86, wherein the dose is 2100 mg, administered once every three weeks.

Embodiment P88: Tire anti-TGFβ agent for use of embodiment P86, wherein the dose is 2400 mg, administered once every three weeks.

Embodiment P89: The anti-TGFβ agent for use of any one of embodiments P51 to P82, wherein the dose is 3000 mg, administered once every three weeks.

Embodiment P90: The anti-TGFβ agent of any one of embodiments P51 to P89, wherein the protein is administered by intravenous administration.

Embodiment P91: The anti-TGFβ agent for use of embodiment P90, wherein the intravenous administration is performed with a prefilled bag, a prefilled pen, or a prefilled syringe comprising a formulation comprising the protein.

Embodiment P92: The anti-TGFβ agent for use of embodiment P91, wherein the bag is connected to a channel comprising a tube and/or a needle.

SEQUENCES
Peptide sequence of the secreted anti-PD-L1 lambda light chain
SEQ ID NO: 1
QSALTQPASVSGSPGQSITISCTGTSSDVGGYNYVSWYQQHPGKAPKLMIYDVSNRPS
GVSNRFSGSKSGNTASLTISGLQAEDEADYYCSSYTSSSTRVFGTGTKVTVLGQPKANP
TVTLFPPSSEELQANKATLVCLISDFYPGAVTVAWKADGSPVKAGVETTKPSKQSNNK
YAASSYLSLTPEQWKSHRSYSCQVTHEGSTVEKTVAPTECS
Peptide sequence of the secreted H chain of anti-PDL1
SEQ ID NO: 2
EVQLLESGGGLVQPGGSLRLSCAASGFTFSSYIMMWVRQAPGKGLEWVSSIYPSGGITF
YADTVKGRFTISRDNSKNTLYLQMNSLRAEDTAVYYCARIKLGTVTTVDYWGQGTLV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Peptide sequence of the secreted H chain of anti-PDL1/TGFβ Trap
SEQ ID NO: 3
EVQLLESGGGLVQPGGSLRLSCAASGFTFSSYIMMWVRQAPGKGLEWVSSIYPSGGITF
YADTVKGRFTISRDNSKNTLYLQMNSLRAEDTAVYYCARIKLGTVTTVDYWGQGTLV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGAGGGGSGGGGSGGGGS
GGGGSGIPPHVQKSVNNDMIVTDNNGAVKFPQLCKFCDVRFSTCDNQKSCMSNCSITS
ICEKPQEVCVAVWRKNDENITLETVCHDPKLPYHDFILEDAASPKCIMKEKKKPGETFF
MCSCSSDECNDNIIFSEEYNTSNPD
DNA sequence from the translation initiation codon to the translation stop codon of the anti-
PD-L1 lambda light chain (the leader sequence preceding the VL is the signal peptide from
urokinase plasminogen activator)
SEQ ID NO: 4
atgagggccctgctggctagactgctgctgtgcgtgctggtcgtgtccgacagcaagggcCAGTCCGCCCTGACCCAG
CCTGCCTCCGTGTCTGGCTCCCCTGGCCAGTCCATCACCATCAGCTGCACCGGCAC
CTCCAGCGACGTGGGCGGCTACAACTACGTGTCCTGGTATCAGCAGCACCCCGGCA
AGGCCCCCAAGCTGATGATCTACGACGTGTCCAACCGGCCCTCCGGCGTGTCCAAC
AGATTCTCCGGCTCCAAGTCCGGCAACACCGCCTCCCTGACCATCAGCGGACTGCA
GGCAGAGGACGAGGCCGACTACTACTGCTCCTCCTACACCTCCTCCAGCACCAGAG
TGTTCGGCACCGGCACAAAAGTGACCGTGCTGggccagcccaaggccaacccaaccgtgacactgttcc
ccccatcctccgaggaactgcaggccaacaaggccaccctggtctgcctgatctcagatttctatccaggcgccgtgaccgtggcctgg
aaggctgatggctccccagtgaaggccggcgtggaaaccaccaagccctccaagcagtccaacaacaaatacgccgcctcctcctacc
tgtccctgacccccgagcagtggaagtcccaccggtcctacagctgccaggtcacacacgagggctccaccgtggaaaagaccgtcg
cccccaccgagtgctcaTGA
DNA sequence from the translation initiation codon to the translation stop codon (mVK SP
leader: small underlined; VH: capitals; IgG1m3 with K to A mutation: small letters; (G4S)x4-G
(SEQ ID NO: 11) linker: bold capital letters; TGFβRII: bold underlined small letters; two stop
codons: bold underlined capital letters)
SEQ ID NO: 5
atggaaacagacaccctgctgctgtgggtgctgctgctgtgggtgcccggctccacaggcGAGGTGCAGCTGCTGGAA
TCCGGCGGAGGACTGGTGCAGCCTGGCGGCTCCCTGAGACTGTCTTGCGCCGCCTC
CGGCTTCACCTTCTCCAGCTACATCATGATGTGGGTGCGACAGGCCCCTGGCAAGG
GCCTGGAATGGGTGTCCTCCATCTACCCCTCCGGCGGCATCACCTTCTACGCCGAC
ACCGTGAAGGGCCGGTTCACCATCTCCCGGGACAACTCCAAGAACACCCTGTACCT
GCAGATGAACTCCCTGCGGGCCGAGGACACCGCCGTGTACTACTGCGCCCGGATC
AAGCTGGGCACCGTGACCACCGTGGACTACTGGGGCCAGGGCACCCTGGTGACAG
TGTCCTCCgctagcaccaagggcccatcggtatccccctggcaccctcctccaagagcacctctgggggcacagcggccctgg
gctgcctggicaaggactacttccccgaaccggtgacggtgtcgtggaactcaggcgccctgaccagcggcgtgcacaccttcccggct
gtcctacagtcctcaggactctactccctcagcagcgtggtgaccgtgccctccagcagcttgggcacccagacctacatctgcaacgtg
aatcacaagcccagcaacaccaaggtggacaagagagttgagcccaaatcttgtgacaaaactcacacatgcccaccgtgcccagcac
ctgaactcctggggggaccgtcagtcttcctcttccccccaaaacccaaggacaccctcatgatctcccggacccctgaggtcacatgcg
tggtggtggacgtgagccacgaagaccctgaggtcaagttcaactggtacgtggacggcgtggaggtgcataatgccaagacaaagcc
gcgggaggagcagtacaacagcacgtaccgtgtggtcagcgtcctcaccgtcctgcaccaggactggctgaatggcaaggagtacaa
gtgcaaggtctccaacaaagccctcccagcccccatcgagaaaaccatctccaaagccaaagggcagccccgagaaccacaggtgta
caccctgcccccatcccgggaggagatgaccaagaaccaggtcagcctgacctgcctggtcaaaggcttctatcccagcgacatcgcc
gtggagtgggagagcaatgggcagccggagaacaactacaagaccacgcctcccgtgctggactccgacggctccttcttcctctatag
caagctcaccgtggacaagagcaggtggcagcaggggaacgtcttctcatgctccgtgatgcatgaggctctgcacaaccactacacg
cagaagagcctctccctgtccccgggtgctGGCGGCGGAGGAAGCGGAGGAGGTGGCAGCGGTG
GCGGTGGCTCCGGCGGAGGTGGCTCCGGAatccctccccacgtgcagaagtccgtgaacaacgac
atgatcgtgaccgacaacaacggcgccgtgaagttccctcagctgtgcaagttctgcgacgtgaggttcagcacctgcgacaacc
agaagtcctgcatgagcaactgcagcatcacaagcatctgcgagaagccccaggaggtgtgtgtggccgtgtggaggaagaac
gacgaaaacatcaccctcgagaccgtgtgccatgaccccaagctgccctaccacgacttcatcctggaagacgccgcctccccc
aagtgcatcatgaaggagaagaagaagcccggcgagaccttcttcatgtgcagctgcagcagcgacgagtgcaatgacaacat
catctttagcgaggagtacaacaccagcaaccccgacTGATAA
Polypeptide sequence of the secreted lambda light chain of anti-PD-L1(mut)/TGFβ Trap, with
mutations A31G, D52E, R99Y
SEQ ID NO: 6
QSALTQPASVSGSPGQSITISCTGTSSDVGGYNYVSWYQQHPGKAPKLMIYEVSNRPSG
VSNRFSGSKSGNTASLTISGLQAEDEADYYCSSYTSSSTYVFGTGTKVTVLGQPKANPT
VTLFPPSSEELQANKATLVCLISDFYPGAVTVAWKADGSPVKAGVETTKPSKQSNNKY
AASSYLSLTPEQWKSHRSYSCQVTHEGSTVEKTVAPTECS
Polypeptide sequence of the secreted heavy chain of anti-PD-L1(mut)/TGFβ Trap
SEQ ID NO: 7
EVQLLESGGGLVQPGGSLRLSCAASGFTFSMYMMMWVRQAPGKGLEWVSSIYPSGGI
TFYADSVKGRFTISRDNSKNTLYLQMNSLRAEDTAIYYCARIKLGTVTTVDYWGQGTL
VTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCP
APELLGGPSVFLEPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGAGGGGSGGGGSGGG
GSGGGGSGIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMKEKKKPGET
FFMCSCSSDECNDNIIFSEEYNTSNPD
Human TGFβRII Isoform A Precursor Polypeptide (NCBI RefSeq Accession No:
NP_001020018)
SEQ ID NO: 8
MGRGLLRGLWPLHIVLWTRIASTIPPHVQKSDVEMEAQKDEIICPSCNRTAHPLRHINN
DMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSE
EYNTSNPDLLLVIFQVTGISLLPPLGVAISVIIIFYCYRVNRQQKLSSTWETGKTRKLMEF
SEHCAIILEDDRSDISSTCANNINHNTELLPIELDTLVGKGRFAEVYKAKLKQNTSEQFE
TVAVKIFPYEEYASWKTEKDIFSDINLKHENILQFLTAEERKTELGKQYWLITAFHAKG
NLQEYLTRHVISWEDLRKLGSSLARGIAHLHSDHTPCGRPKMPIVHRDLKSSNILVKND
LTCCLCDFGLSLRLDPTLSVDDLANSGQVGTARYMAPEVLESRMNLENVESFKQTDV
YSMALVLWEMTSRCNAVGEVKDYEPPFGSKVREHPCVESMKDNVLRDRGRPEIPSFW
LNHQGIQMVCETLTECWDHDPEARLTAQCVAERFSELEHLDRLSGRSCSEEKIPEDGSL
NTTK
Human TGFβRII Isoform B Precursor Polypeptide (NCBI RefSeq Accession No: NP_003233)
SEQ ID NO: 9
MGRGLLRGLWPLHIVLWTRIASTIPPHVQKSVNNDMIVTDNNGAVKFPQLCKFCDVRF
STCDNQKSCMSNCSITSICEKPQEVCVAVWRKNDENITLETVCHDPKLPYHDFILEDAA
SPKCIMKEKKKPGETFFMCSCSSDECNDNIIFSEEYNTSNPDLLLVIFQVTGISLLPPLGV
AISVIIIFYCYRVNRQQKLSSTWETGKTRKLMEFSEHCAIILEDDRSDISSTCANNINHNT
ELLPIELDTLVGKGRFAEVYKAKLKQNTSEQFETVAVKIFPYEEYASWKTEKDIFSDIN
LKHENILQFLTAEERKTELGKQYWLITAFHAKGNLQEYLTRHVISWEDLRKLGSSLAR
GIAHLHSDHTPCGRPKMPIVHRDLKSSNILVKNDLTCCLCDFGLSLRLDPTLSVDDLAN
SGQVGTARYMAPEVLESRMNLENVESFKQTDVYSMALVLWEMTSRCNAVGEVKDYE
PPFGSKVREHPCVESMKDNVLRDRGRPEIPSFWLNHQGIQMVCETLTECWDHDPEARL
TAQCVAERFSELEHLDRLSGRSCSEEKIPEDGSLNTTK
A Human TGFβRII Isoform B Extracellular Domain Polypeptide
SEQ ID NO: 10
IPPHVQKSVNNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGETFFMCSCSS
DECNDNIIFSEEYNTSNPD
(Gly₄Ser)₄Gly linker
SEQ ID NO: 11
GGGGSGGGGSGGGGSGGGGSG
Polypeptide sequence of the secreted heavy chain variable region of anti-PD-L1 antibody
MPDL3289A
SEQ ID NO: 12
EVQLVESGGGLVQPGGSLRLSCAASGFTFSDSWIHWVRQAPGKGLEWVAWISPYGGS
TYYADSVKGRFTISADTSKNTAYLQMNSLRAEDTAVYYCARRHWPGGFDYWGQGTL
VTVSS
Polypeptide sequence of the secreted light chain variable region of anti-PD-L1 antibody
MPDL3289A
SEQ ID NO: 13
DIQMTQSPSSLSASVGDRVTITCRASQDVSTAVAWYQQKPGKAPKLLIYSASFLYSGVP
SRFSGSGSGTDFTLTISSLQPEDFATYYCQQYLYHPATFGQGTKVEIKR
Polypeptide sequence of the secreted heavy chain variable region of anti-PD-L1 antibody
YW243.55S70
SEQ ID NO: 14
EVQLVESGGGLVQPGGSLRLSCAASGFTFSDSWIHWVRQAPGKGLEWVAWISPYGGS
TYYADSVKGRFTISADTSKNTAYLQMNSLRAEDTAVYYCARRHWPGGFDYWGQGTL
VTVSA
A Truncated Human TGFβRII Isoform B Extracellular Domain Polypeptide
SEQ ID NO: 50
GAVKFPQLCKFCDVRFSTCDNQKSCMSNCSITSICEKPQEVCVAVWRKNDENITLETV
CHDPKLPYHDFILEDAASPKCIMKEKKKPGETFFMCSCSSDECNDNIIFSEEYNTSNPD
A Truncated Human TGFβRII Isoform B Extracellular Domain Polypeptide
SEQ ID NO: 51
VKFPQLCKFCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEEYNTSNPD
A Truncated Human TGFβRII Isoform B Extracellular Domain Polypeptide
SEQ ID NO: 52
VTDNNGAVKFPQLCKFCDVRFSTCDNQKSCMSNCSITSICEKPQEVCVAVWRKNDENI
TLETVCHDPKLPYHDFILEDAASPKCIMKEKKKPGETFFMCSCSSDECNDNIIFSEEYNT
SNPD
A Truncated Human TGFβRII Isoform B Extracellular Domain Polypeptide
SEQ ID NO: 53
LCKFCDVRFSTCDNQKSCMSNCSITSICEKPQEVCVAVWRKNDENITLETVCHDPKLP
YHDFILEDAASPKCIMKEKKKPGETFFMCSCSSDECNDNIIFSEEYNTSNPD
A Mutated Human TGFβRII Isoform B Extracellular Domain Polypeptide
SEQ ID NO: 54
VTDNAGAVKFPQLCKFCDVRESTCDNQKSCMSNCSITSICEKPQEVCVAVWRKNDENI
TLETVCHDPKLPYHDFILEDAASPKCIMKEKKKPGETFFMCSCSSDECNDNIIFSEEYNT
SNPD
Polypeptide sequence of the heavy chain variable region of anti-PD-L1 antibody
SEQ ID NO: 55
QVQLQESGPGLVKPSQTLSLTCTVSGGSISNDYWTWIRQHPGKGLEYIGYISYTGSTYY
NPSLKSRVTISRDTSKNQFSLKLSSVTAADTAVYYCARSGGWLAPFDYWGRGTLVTVS
S
Polypeptide sequence of the light chain variable region of anti-PD-L1 antibody
SEQ ID NO: 56
DIVMTQSPDSLAVSLGERATINCKSSQSLEVHSNQKHSLAWYQQKPGQPPKLLIYGAST
RESGVPDRFSGSGSGTDFTLTISSLQAEDVAVYYCQQYYGYPYTFGGGTKVEIK
Polypeptide sequence of the heavy chain variable region of anti-PD-L1 antibody
SEQ ID NO: 57
QVQLVQSGAEVKKPGASVKVSCKASGYTFTSYWMHWVRQAPGQGLEWMGRIGPNS
GFTSYNEKFKNRVTMTRDTSTSTVYMELSSLRSEDTAVYYCARGGSSYDYFDYWGQG
TTVTVSS
Polypeptide sequence of the light chain variable region of anti-PD-L1 antibody
SEQ ID NO: 58
DIVLTQSPASLAVSPGQRATITCRASESVSIHGTHLMHWYQQKPGQPPKLLIYAASNLE
SGVPARFSGSGSGTDFTLTINPVEAEDTANYYCQQSFEDPLTFGQGTKLEIK
Polypeptide sequence of the heavy chain of anti-PD-L1 antibody
SEQ ID NO: 59
QVQLQESGPGLVKPSQTLSLTCTVSGGSISNDYWTWIRQHPGKGLEYIGYISYTGSTYY
NPSLKSRVTISRDTSKNQFSLKLSSVTAADTAVYYCARSGGWLAPFDYWGRGTLVTVS
SASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEAAGG
PSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK
Polypeptide sequence of the light chain of anti-PD-L1 antibody
SEQ ID NO: 60
DIVMTQSPDSLAVSLGERATINCKSSQSLFYHSNQKHSLAWYQQKPGQPPKLLIYGAST
RESGVPDRFSGSGSGTDFTLTISSLQAEDVAVYYCQQYYGYPYTFGGGTKVEIKRTVA
APSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
Polypeptide sequence of the heavy chain of anti-PD-L1 antibody
SEQ ID NO: 61
QVQLVQSGAEVKKPGASVKVSCKASGYTFTSYWMHWVRQAPGQGLEWMGRIGPNS
GFTSYNEKFKNRVTMTRDTSTSTVYMELSSLRSEDTAVYYCARGGSSYDYFDYWGQG
TTVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPA
PEAAGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGA
Polypeptide sequence of the light chain of anti-PD-L1 antibody
SEQ ID NO: 62
DIVLTQSPASLAVSPGQRATITCRASESVSIHGTHLMHWYQQKPGQPPKLLIYAASNLE
SGVPARFSGSGSGTDFTLTINPVEAEDTANYYCQQSFEDPLTFGQGTKLEIKRTVAAPS
VFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
HMGA2 variant 1 RefSeq NM_003483.4
>NM_003483.4 Homo sapiens high mobility group AT-hook 2 (HMGA2), transcript variant 1,
mRNA
SEQ ID NO: 63
CTTGAATCTTGGGGCAGGAACTCAGAAAACTTCCAGCCCGGGCAGCGCGCGCTTG
GTGCAAGACTCAGGAGCTAGCAGCCCGTCCCCCTCCGACTCTCCGGTGCCGCCGCT
GCCTGCTCCCGCCACCCTAGGAGGCGCGGTGCCACCCACTACTCTGTCCTCTGCCT
GTGCTCCGTGCCCGACCCTATCCCGGCGGAGTCTCCCCATCCTCCTTTGCTITCCGA
CTGCCCAAGGCACTTTCAATCTCAATCTCTTCTCTCTCTCTCTCTCTCTCTCTCTC
TCTCTCTCTCTCTCTCTCTCTCTCTCTCTCGCAGGGTGGGGGGAAGAGGAGGAGGA
ATTCTTTCCCCGCCTAACATTTCAAGGGACACAATTCACTCCAAGTCTCTTCCCTTT
CCAAGCCGCTTCCGAAGTGCTCCCGGTGCCCGCAACTCCTGATCCCAACCCGCGAG
AGGAGCCTCTGCGACCTCAAAGCCTCTCTTCCTTCTCCCTCGCTTCCCTCCTCCTCT
TGCTACCTCCACCTCCACCGCCACCTCCACCTCCGGCACCCACCCACCGCCGCCGC
CGCCACCGGCAGCGCCTCCTCCTCTCCTCCTCCTCCTCCCCTCTTCTCTTTTTGGCAG
CCGCTGGACGTCCGGTGTTGATGGTGGCAGCGGCGGCAGCCTAAGCAACAGCAGC
CCTCGCAGCCCGCCAGCTCGCGCTCGCCCCGCCGGCGTCCCCAGCCCTATCACCTC
ATCTCCCGAAAGGTGCTGGGCAGCTCCGGGGCGGTCGAGGCGAAGCGGCTGCAGC
GGCGGTAGCGGCGGCGGGAGGCAGGATGAGCGCACGCGGTGAGGGCGCGGGGCA
GCCGTCCACTTCAGCCCAGGGACAACCTGCCGCCCCAGCGCCTCAGAAGAGAGGA
CGCGGCCGCCCCAGGAAGCAGCAGCAAGAACCAACCGGTGAGCCCTCTCCTAAGA
GACCCAGGGGAAGACCCAAAGGCAGCAAAAACAAGAGTCCCTCTAAAGCAGCTCA
AAAGAAAGCAGAAGCCACTGGAGAAAAACGGCCAAGAGGCAGACCTAGGAAATG
GCCACAACAAGTTGTTCAGAAGAAGCCTGCTCAGGAGGAAACTGAAGAGACATCC
TCACAAGAGTCTGCCGAAGAGGACTAGGGGGCGCCAACGTTCGATTTCTACCTCAG
CAGCAGTTGGATCTTTTGAAGGGAGAAGACACTGCAGTGACCACTTATTCTGTATT
GCCATGGTCTTTCCACTTTCATCTGGGGTGGGGTGGGGTGGGGTGGGGGAGGGGG
GGGTGGGGTGGGGAGAAATCACATAACCTTAAAAAGGACTATATTAATCACCTTCT
TTGTAATCCCTTCACAGTCCCAGGTTTAGTGAAAAACTGCTGTAAACACAGGGGAC
ACAGCTTAACAATGCAACTTTTAATTACTGTTTTCTTTTTTCTTAACCTACTAATAGT
TTGTTGATCTGATAAGCAAGAGTGGGCGGGTGAGAAAAACCGAATTGGGTTTAGT
CAATCACTGCACTGCATGCAAACAAGAAACGTGTCACACTTGTGACGTCGGGCATT
CATATAGGAAGAACGCGGTGTGTAACACTGTGTACACCTCAAATACCACCCCAACC
CACTCCCTGTAGTGAATCCTCTGTTTAGAACACCAAAGATAAGGACTAGATACTAC
TTTCTCTTTTTCGTATAATCTTGTAGACACTTACTTGATGATTTTTAACTTTTTATTT
CTAAATGAGACGAAATGCTGATGTATCCTTTCATTCAGCTAACAAACTAGAAAAGG
TTATGTTCATTTTTCAAAAAGGGAAGTAAGCAAACAAATATTGCCAACTCTTCTAT
TTATGGATATCACACATATCAGCAGGAGTAATAAATTTACTCACAGCACTTGTTTT
CAGGACAACACTTCATTTTCAGGAAATCTACTTCCTACAGAGCCAAAATGCCATTT
AGCAATAAATAACACTTGTCAGCCTCAGAGCATTTAAGGAAACTAGACAAGTAAA
ATTATCCTCTTTGTAATTTAATGAAAAGGTACAACAGAATAATGCATGATGAACTC
ACCTAATTATGAGGTGGGAGGAGCGAAATCTAAATTTCTTTTGCTATAGTTATACA
TCAATTTAAAAAGCAAAAAAAAAAAAGGGGGGGGCAATCTCTCTCTGTGTCTTTCT
CTCTCTCTCTTCCTCTCCCTCTCTCTTTTCATTGTGTATCAGTTTCCATGAAAGACCT
GAATACCACTTACCTCAAATTAAGCATATGTGTTACTTCAAGTAATACGTTTTGAC
ATAAGATGGTTGACCAAGGTGCTTTTCTTCGGCTTGAGTTCACCATCTCTTCATTCA
AACTGCACTTTTAGCCAGAGATGCAATATATCCCCACTACTCAATACTACCTCTGA
ATGTTACAACGAATTTACAGTCTAGTACTTATTACATGCTGCTATACACAAGCAAT
GCAAGAAAAAAACTTACTGGGTAGGTGATTCTAATCATCTGCAGTTCTTTTTGTAC
ACTTAATTACAGTTAAAGAAGCAATCTCCTTACTGTGTTTCAGCATGACTATGTATT
TTTCTATGTTTTTTTAATTAAAAATTTTTAAAATACTTGTTTCAGCTTCTCTGCTAGA
TTTCTACATTAACTTGAAAATTTTTTAACCAAGTCGCTCCTAGGTTCTTAAGGATAA
TTTTCCTCAATCACACTACACATCACACAAGATTTGACTGTAATATTTAAATATTAC
CCTCCAAGTCTGTACCTCAAATGAATTCTTTAAGGAGATGGACTAATTGACTTGCA
AAGACCTACCTCCAGACTTCAAAAGGAATGAACTTGTTACTTGCAGCATTCATTTG
TTTTTTCAATGTTTGAAATAGTTCAAACTGCAGCTAACCCTAGTCAAAACTATTTTT
GTAAAAGACATTTGATAGAAAGGAACACGTTTTTACATACTTTTGCAAAATAAGTA
AATAATAAATAAAATAAAAGCCAACCTTCAAAGAAACTTGAAGCTTTGTAGGTGA
GATGCAACAAGCCCTGCTTTTGCATAATGCAATCAAAAATATGTGTTTTTAAGATT
AGTTGAATATAAGAAAATGCTTGACAAATATTTTCATGTATITTACACAAATGTGA
TTTTTGTAATATGTCTCAACCAGATTTATTTTAAACGCTTCTTATGTAGAGTTTTTAT
GCCTTTCTCTCCTAGTGAGTGTGCTGACTTTTTAACATGGTATTATCAACTGGGCCA
GGAGGTAGTTTCTCATGACGGCTTTTGTCAGTATGGCTTTTAGTACTGAAGCCAAA
TGAAACTCAAAACCATCTCTCTTCCAGCTGCTTCAGGGAGGTAGTTTCAAAGGCCA
CATACCTCTCTGAGACTGGCAGATCGCTCACTGTTGTGAATCACCAAAGGAGCTAT
GGAGAGAATTAAAACTCAACATTACTGTTAACTGTGCGTTAAATAAGCAAATAAA
CAGTGGCTCATAAAAATAAAAGTCGCATTCCATATCTTTGGATGGGCCTTTTAGAA
ACCTCATTGGCCAGCTCATAAAATGGAAGCAATTGCTCATGTTGGCCAAACATGGT
GCACCGAGTGATTTCCATCTCTGGTAAAGTTACACTTTTATTTCCTGTATGTTGTAC
AATCAAAACACACTACTACCTCTTAAGTCCCAGTATACCTCATTTTTCATACTGAAA
AAAAAAGCTTGTGGCCAATGGAACAGTAAGAACATCATAAAATTTTTATATATATA
GTTTATTTTTGTGGGAGATAAATTTTATAGGACTGTTCTTTGCTGTTGTTGGTCGCA
GCTACATAAGACTGGACATTTAACTTTTCTACCATTTCTGCAAGTTAGGTATGTTTG
CAGGAGAAAAGTATCAAGACGTTTAACTGCAGTTGACTTTCTCCCTGTTCCTTTGA
GTGTCTTCTAACTTTATTCTTTGTTCTTTATGTAGAATTGCTGTCTATGATTGTACTT
TGAATCGCTTGCTTGTTGAAAATATTTCTCTAGTGTATTATCACTGTCTGTTCTGCA
CAATAAACATAACAGCCTCTGTGATCCCCATGTGTTTTGATTCCTGCTCTTTGTTAC
AGTTCCATTAAATGAGTAATAAAGTTTGGTCAAAACAGAAAAAAAAAAA
MECOM RefSeq NM_001105077.3
>NM_001105077.3 Homo sapiens MDS1 and EVI1 complex locus (MECOM), transcript
variant 1, mRNA
SEQ ID NO: 64
CCTTGCCAAGTAACAGCTTTGCTGTCCAACATCGTGTGCTGCTTCGCGAGAAAGTC
ACATTCGGACCCTTTGGCTAGATTGCTTATTCATAGGGCTTCTTGACTAAAGCCCTT
GGAGCACTGGGTTTTTCTTGAAGTATATGATCTTAGACGAATTTTACAATGTGAAG
TTCTGCATAGATGCCAGTCAACCAGATGTTGGAAGCTGGCTCAAGTACATTAGATT
CGCTGGCTGTTATGATCAGCACAACCTTGTTGCATGCCAGATAAATGATCAGATA
TTCTATAGAGTAGTTGCAGACATTGCGCCGGGAGAGGAGCTTCTGCTGTTCATGAA
GAGCGAAGACTATCCCCATGAAACTATGGCGCCGGATATCCACGAAGAACGGCAA
TATCGCTGCGAAGACTGTGACCAGCTCTTTGAATCTAAGGCTGAACTAGCAGATCA
CCAAAAGTTTCCATGCAGTACTCCTCACTCAGCATTTTCAATGGTTGAAGAGGACT
TTCAGCAAAAACTCGAAAGCGAGAATGATCTCCAAGAGATACACACGATCCAGGA
GTGTAAGGAATGTGACCAAGTTTTTCCTGATTTGCAAAGCCTGGAGAAACACATGC
TGTCACATACTGAAGAGAGGGAATACAAGTGTGATCAGTGTCCCAAGGCATTTAA
CTGGAAGTCCAATTTAATTCGCCACCAGATGTCACATGACAGTGGAAAGCACTATG
AATGTGAAAACTGTGCCAAGCAGGTTTTCACGGACCCTAGCAACCTTCAGCGGCAC
ATTCGCTCTCAGCATGTCGGTGCCCGGGCCCATGCATGCCCGGAGTGTGGCAAAAC
GTTTGCCACTTCGTCGGGCCTCAAACAACACAAGCACATCCACAGCAGTGTGAAGC
CCTTTATCTGTGAGGTCTGCCATAAATCCTATACTCAGTTTTCAAACCTTTGCCGTC
ATAAGCGCATGCATGCTGATTGCAGAACCCAAATCAAGTGCAAAGACTGTGGACA
AATGTTCAGCACTACGTCTTCCTTAAATAAACACAGGAGGTTTTGTGAGGGCAAGA
ACCATTTTGCGGCAGGTGGATTTTTTGGCCAAGGCATTTCACTTCCTGGAACCCCA
GCTATGGATAAAACGTCCATGGTTAATATGAGTCATGCCAACCCGGGCCTTGCTGA
CTATTTTGGCGCCAATAGGCATCCTGCTGGTCTTACCTTTCCAACAGCTCCTGGATT
TTCTTTTAGCTTCCCTGGTCTGTTTCCTTCCGGCTTGTACCACAGGCCTCCTTTGATA
CCTGCTAGTTCTCCTGTTAAAGGACTATCAAGTACTGAACAGACAAACAAAAGTCA
AAGTCCCCTCATGACACATCCTCAGATACTGCCAGCTACACAGGATATTTTGAAGG
CACTATCTAAACACCCATCTGTAGGGGACAATAAGCCAGTGGAGCTCCAGCCCGA
GAGGTCCTCTGAAGAGAGGCCCTTTGAGAAAATCAGTGACCAGTCAGAGAGTAGT
GACCTTGATGATGTCAGTACACCAAGTGGCAGTGACCTGGAAACAACCTCGGGCTC
TGATCTGGAAAGTGACATTGAAAGTGATAAAGAGAAATTTAAAGAAAATGGTAAA
ATGTTCAAAGACAAAGTAAGCCCTCTTCAGAATCTGGCTTCAATAAATAATAAGAA
AGAATACAGCAATCATTCCATTTTCTCACCATCTTTAGAGGAGCAGACTGCGGTGT
CAGGAGCTGTGAATGATTCTATAAAGGCTATTGCTTCTATTGCTGAAAAATACTTT
GGTTCAACAGGACTGGTGGGGCTGCAAGACAAAAAAGTTGGAGCTTTACCTTACC
CTTCCATGTTTCCCCTCCCATTTTTTCCAGCATTCTCTCAATCAATGTACCCATTTCC
TGATAGAGACTTGAGATCGTTACCTTTGAAAATGGAACCCCAATCACCAGGTGAAG
TAAAGAAACTGCAGAAGGGCAGCTCTGAGTCCCCCTTTGATCTCACCACTAAGCGA
AAGGATGAGAAGCCCTTGACTCCAGTCCCCTCCAAGCCTCCAGTGACACCTGCCAC
AAGCCAAGACCAGCCCCTGGATCTAAGTATGGGCAGTAGGAGTAGAGCCAGTGGG
ACAAAGCTGACTGAGCCTCGAAAAAACCACGTGTTTGGGGGAAAAAAAGGAAGCA
ACGTCGAATCAAGACCTGCTTCAGATGGTTCCTTGCAGCATGCAAGACCCACTCCT
TTCTTTATGGACCCTATTTACAGAGTAGAGAAAAGAAAACTAACTGACCCACTTGA
AGCTTTAAAAGAGAAATACTTGAGGCCTTCTCCAGGATTCTTGTTTCACCCACAAT
TCCAACTGCCTGATCAGAGAACTTGGATGTCAGCTATTGAAAACATGGCAGAAAA
GCTAGAGAGCTTCAGTGCCCTGAAACCTGAGGCCAGTGAGCTCTTACAGTCAGTGC
CCTCTATGTTCAACTTCAGGGCGCCTCCCAATGCCCTGCCAGAGAACCTTCTGCGG
AAGGGAAAGGAGCGCTATACCTGCAGATACTGTGGCAAGATTTTTCCAAGGTCTGC
AAACCTAACACGGCACTTGAGAACCCACACAGGAGAGCAGCCTTACAGATGCAAA
TACTGTGACAGATCATTTAGCATATCTTCTAACTTGCAAAGGCATGTTCGCAACAT
CCACAATAAAGAGAAGCCATTTAAGTGTCACTTATGTGATAGGTGTTTTGGTCAAC
AAACCAATTTAGACAGACACCTAAAGAAACATGAGAATGGGAACATGTCCGGTAC
AGCAACATCGTCGCCTCATTCTGAACTGGAAAGTACAGGTGCGATTCTGGATGACA
AAGAAGATGCTTACTTCACAGAAATTCGAAATTTCATTGGGAACAGCAACCATGGC
AGCCAATCTCCCAGGAATGTGGAGGAGAGAATGAATGGCAGTCATTTTAAAGATG
AAAAGGCTTTGGTGACCAGTCAAAATTCAGACTTGCTGGATGATGAAGAAGTTGA
AGATGAGGTGITGTTAGATGAGGAGGATGAAGACAATGATATTACTGGAAAAACA
GGAAAGGAACCAGTGACAAGTAATTTACATGAAGGAAACCCTGAGGATGACTATG
AAGAAACCAGTGCCCTGGAGATGAGTTGCAAGACATCCCCAGTGAGGTATAAAGA
GGAAGAATATAAAAGTGGACTTTCTGCTCTAGATCATATAAGGCACTTCACAGATA
GCCTCAAAATGAGGAAAATGGAAGATAATCAATATTCTGAAGCTGAGCTGTCTT
CTTTTAGTACTTCCCATGTGCCAGAGGAACTTAAGCAGCCGTTACACAGAAAGTCC
AAATCGCAGGCATATGCTATGATGCTGTCACTGTCTGACAAGGAGTCCCTCCATTC
TACATCCCACAGTTCTTCCAACGTGTGGCACAGTATGGCCAGGGCTGCGGCGGAAT
CCAGTGCTATCCAGTCCATAAGCCACGTATGACGTTATCAAGGTTGACCAGAGTGG
GACCAAGTCCAACAGTAGCATGGCTCTTTCATATAGGACTATTTACAAGACTGCTG
AGCAGAATGCCTTATAAACCTGCAGGGTCACTCATCTAAAGTCTAGTGACCTTAAA
CTGAATGATTTAAAAAAGAAAAGAAAGAAAAAAGAAACTATTTATTCTCGATATT
TTGTTTTGCACAGCAAAGGCAGCTGCTGACTTCTGGAAGATCAATCAATGCGACTT
AAAGTGATTCAGTGAAAACAAAAAACTTGGTGGGCTGAAGGCATCTTCCAGTTTAC
CCCACCTTAGGGTATGGGTGGGTGAGAAGGGCAGTTGAGATGGCAGCATTGATAT
GAATGAACACTCCATAGAAACTGAATTCTCTTTTGTACAAGATCACCTGACATGAT
TGGGAACAGTTGCTTTTAATTACAGATTTAATTTTTTTCTTCGTTAAAGTTTTATGTA
ATTTAACCCTTTGAAGACAGAAGTAGTTGGATGAAATGCACAGTCAATTATTATAG
AAACTGATAACAGGGAGTACTTGTTCCCCCTTTTGCCTTCTTAAGTACATTGTTTAA
AACTAGGGAAAAAGGGTATGTGTATATTGTAAACTATGGATGTTAACACTCAAAG
AGGTTAAGTCAGTGAAGTAACCTATTCATCACCAGTACCGCTGTACCACTAATAAA
TTGTTTGCCAAATCCTTGTAATAACATCTTAATTTTAGACAATCATGTCACTGTTTT
TAATGTTTATTTTTTTGTGTGTGTTGCGTGTATCATGTATTTATTTGTTGGCAAACTA
TTGTTTGTTGATTAAAATAGCACTGTTCCAGTCAGCCACTACTTTATGACGTCTGAG
GCACACCCCTTTCCGAATTTCAAGGACCAAGGTGACCCGACCTGTGTATGAGAGTG
CCAAATGGTGTTTGGCTTTTCTTAACATTCCTTTTTGTTTGTTTGTTTTGTTTTCCTTC
TTAATGAACTAAATACGAATAGATGCAACTTAGTTTTTGTAATACTGAAATCGATT
CAATTGTATAAACGATTATAATTTCTTTCATGGAAGCATGATTCTTCTGATTAAAAA
CTGTACTCCATATTTTATGCTGGTTGTCTGCAAGCTTGTGCGATGTTATGTTCATGT
TAATCCTATTTGTAAAATGAAGTGTTCCCAACCTTATGTTAAAAGAGAGAAGTAAA
TAACAGACTGTATTCAGTTATTTTGCCCTTTATTGAGGAACCAGATTTGTTTTCTTTT
TGTTTGTAATCTCATTTTGAAATAATCAGCAAGTTGAGGTACTTTCTTCAAATGCTT
TGTACAATATAAACTGTTATGCCTTTCAGTGCATTACTATGGGAGGAGCAACTAAA
AAATAAAGACTTACAAAAAGGAGTATTTTT
synthetic construct
SEQ ID NO: 65
GCGAAGCGGCTGCAGCGGCGGTAGCGGCGGCGGGAGGCAGGATGAGCGCACGCG
GTGAGGGCGCGGGGCAGCCGTCCACTTCAGCCCAGGGACAACCTGCCGCCCCAGC
GCCTCAGAAGAGAGGACGCGGCCGCCCCAGGAAGCAGCAGCAAGAACCAACCGG
TGAGCCCTCTCCTAAGAGACCCAGGGGAAGACCCAAAGGCAGCAAAAACAAGAGT
CCCTCTAAAGCAGCTCAAAAGAAAGCAGAAGCCACTGGAGAAAAACGGCCAAGA
GGCAGACCTAGGAAATGGCCACAACAAGTTGTTCAGAAGAAGCCTGCTCAGGTCA
ATGTTGCCTTGCCTGGGAAGGACCACCCGGGCAATCTTATATATCTACTGTTCTCTA
AA
>NP_003474.1 high mobility group protein HMGI-C isoform a [Homo sapiens]
SEQ ID NO: 66
MSARGEGAGQPSTSAQGQPAAPAPQKRGRGRPRKQQQEPTGEPSPKRPRGRPKGSKN
KSPSKAAQKKAEATGEKRPRGRPRKWPQQVVQKKPAQEETEETSSQESAEED
>NP_001098547.3 MDS1 and EVI1 complex locus protein isoform a [Homo sapiens]
SEQ ID NO: 67
MILDEFYNVKFCIDASQPDVGSWLKYIRFAGCYDQHNLVACQINDQIFYRVVADIAPG
EELLLFMKSEDYPHETMAPDIHEERQYRCEDCDQLFESKAELADHQKFPCSTPHSAFS
MVEEDFQQKLESENDLQEIHTIQECKECDQVFPDLQSLEKHMLSHTEEREYKCDQCPK
AFNWKSNLIRHQMSHDSGKHYECENCAKQVFTDPSNLQRHIRSQHVGARAHACPECG
KTFATSSGLKQHKHIHSSVKPFICEVCHKSYTQFSNLCRHKRMHADCRTQIKCKDCGQ
MFSTTSSLNKHRRFCEGKNHFAAGGFFGQGISLPGTPAMDKTSMVNMSHANPGLADY
FGANRHPAGLTFPTAPGFSFSFPGLFPSGLYHRPPLIPASSPVKGLSSTEQTNKSQSPLMT
HPQILPATQDILKALSKHPSVGDNKPVELQPERSSEERPFEKISDQSESSDLDDVSTPSGS
DLETTSGSDLESDIESDKEKFKENGKMFKDKVSPLQNLASINNKKEYSNHSIFSPSLEEQ
TAVSGAVNDSIKAIASIAEKYFGSTGLVGLQDKKVGALPYPSMFPLPFFPAFSQSMYPF
PDRDLRSLPLKMEPQSPGEVKKLQKGSSESPFDLTTKRKDEKPLTPVPSKPPVTPATSQ
DQPLDLSMGSRSRASGTKLTEPRKNHVFGGKKGSNVESRPASDGSLQHARPTPFFMDP
IYRVEKRKLTDPLEALKEKYLRPSPGFLFHPQFQLPDQRTWMSAIENMAEKLESFSALK
PEASELLQSVPSMFNFRAPPNALPENLLRKGKERYTCRYCGKIFPRSANLTRHLRTHTG
EQPYRCKYCDRSFSISSNLQRHVRNIHNKEKPFKCHLCDRCFGQQTNLDRHLKKHENG
NMSGTATSSPHSELESTGAILDDKEDAYFTEIRNFIGNSNHGSQSPRNVEERMNGSHFK
DEKALVTSQNSDLLDDEEVEDEVLLDEEDEDNDITGKTGKEPVTSNLHEGNPEDDYEE
TSALEMSCKTSPVRYKEEEYKSGLSALDHIRHFTDSLKMRKMEDNQYSEAELSSFSTS
HVPEELKQPLHRKSKSQAYAMMLSLSDKESLHSTSHSSSNVWHSMARAAAESSAIQSI
SHV

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. A method of treating or managing triple negative breast cancer (TNBC) in a patient, the method comprising administering an anti-TGFβ agent to a patient who has been determined to have an increased level of high mobility group AT-hook 2 (HMGA2) expression relative to a known control level, and thereby treating TNBC in the patient.

2. A method of achieving at least a partial response in treating or increasing survival of a triple negative breast cancer (TNBC) patient, the method comprising administering an anti-TGFβ agent to a patient who has been determined to have an increased level of high mobility group AT-hook 2 (HMGA2) expression relative to a known control level, and thereby achieving at least a partial response in treating TNBC in the patient.

3. A method of identifying a patient suitable for treating or managing triple negative breast cancer (TNBC) in the patient with an anti-TGFβ agent, the method comprising determining the level of high mobility group AT-hook 2 (HMGA2) in the patient, wherein an increased level of HMGA2 expression in the patient, relative to a known control level, identifies the patient as suitable for treating TNBC with said anti-TGFβ agent.

4. The method of any one of claims 1 to 3, wherein the HMGA2 level of the patient is determined by analyzing a tissue sample from the patient.

5. The method of claim 4, wherein the tissue sample is a biopsy sample, blood, serum, or plasma sample.

6. The method of claim 4 or 5, wherein the level of HMGA2 is determined by immunochemistry or by RNA expression analysis.

7. The method of any one of claims 1 to 6, wherein the anti-TGFβ agent is an anti-PD-L1/TGFβ Trap protein comprising a first polypeptide comprising: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), and a second polypeptide comprising at least a variable region of a light chain of an antibody that binds PD-L1; wherein the heavy chain of the first polypeptide and the light chain of the second polypeptide, when combined, form an antigen binding site that binds PD-L1.

8. The method of claim 7, wherein the first polypeptide comprises the amino acid sequences of SEQ ID NOs: 35, 36, and 37, and the second polypeptide comprises the amino acid sequences of SEQ ID NOs: 38, 39, and 40.

9. The method of claim 7 or 8, wherein the first polypeptide comprises the amino acid sequence of SEQ ID NO: 3, and the second polypeptide comprises the amino acid sequence of SEQ TD NO: 1.

10. The method of any one of claims 7 to 9, wherein the patient is administered at least 1200 mg of the anti-PD-L1/TGFβ Trap protein.

11. The method of any one of claims 7 to 9, wherein the patient is administered at least 1800 mg of the anti-PD-L1/TGFβ Trap protein.

12. The method of any one of claims 7 to 9, wherein the patient is administered 1800 mg to 3000 mg of the anti-PD-L1/TGFβ Trap protein.

13. The method of any one of claims 7 to 9, wherein the patient is administered 1800 mg to 2100 mg of the anti-PD-L1/TGFβ Trap protein.

14. The method of any one of claims 7 to 9, wherein the patient is administered 1200 mg of the anti-PD-L1/TGFβ Trap protein.

15. The method of claim 14, wherein the patient is administered 1200 mg of the anti-PD-L1/TGFβ Trap protein, once every two weeks.

16. The method of claim 12, wherein the patient is administered 2400 mg of the anti-PD-L1/TGFβ Trap protein.

17. The method of claim 16, wherein the patient is administered 2400 mg of the anti-PD-L1/TGFβ Trap protein, once every three weeks.

18. The method of claim 12, wherein the patient is administered 2100 mg or 3000 mg of the anti-PD-L1/TGFβ Trap protein, once every three weeks.

19. The method of any one of claims 1 to 18, wherein the increased HMGA2 expression has been determined via quantification of HMGA2 mRNA expression.

20. The method of claim 19, wherein the quantification of HMGA2 mRNA expression is via PCR.

21. The method of any one of claims 1 to 20, wherein the increased HMGA2 expression is at least 2.27-fold more than a known population mean HMGA2 expression among TNBC patients.

22. The method of any one of claims 1 to 21, wherein the increased HMGA2 expression is at least 5-fold more than a known population mean HMGA2 expression among TNBC patients.

23. The method of any one of claims 1 to 20, wherein the increased HMGA2 expression is at least 200% higher, at least 300% higher, at least 400% higher, at least 500% higher, at least 600% higher, at least 700% higher, at least 800% higher, at least 900% higher, at least 1000% higher, or more than the normal level of HMGA2 expression.

24. The method of any one of claims 1 to 20, wherein the increased HMGA2 expression in the patient is at least 19- to 35-fold more than the HMGA2 expression in a patient who is non-responsive to a treatment with the anti-TGFβ agent.

25. The method of any one of claims 1 to 18, wherein the increased HMGA2 expression has been determined by HMGA2 protein expression level.

26. The method of claim 25, wherein the increased HMGA2 protein expression level has been determined via immunohistochemistry.

27. The method of claim 26, wherein more than 1% tumor cells expressing HMGA2 protein in a tissue sample obtained from the TNBC patient determined the increased HMGA2 protein expression level.

28. An anti-TGFβ agent for use in a method of treating or managing triple negative breast cancer (TNBC) in a patient, the method comprising administering an anti-TGFβ agent to a patient who has been determined to have an increased level of high mobility group AT-hook 2 (HMGA2) expression relative to a known control level, and thereby treating TNBC in the patient.

29. An anti-TGFβ agent for use in a method of achieving at least a partial response in treating or increasing survival of a triple negative breast cancer (TNBC) patient, the method comprising administering an anti-TGFβ agent to a patient who has been determined to have an increased level of high mobility group AT-hook 2 (HMGA2) expression relative to a known control level, and thereby achieving at least a partial response in treating TNBC in the patient.

30. An anti-TGFβ agent for use in a method of identifying a patient suitable for treating or managing triple negative breast cancer (TNBC) in the patient with an anti-TGFβ agent, the method comprising determining the level of high mobility group AT-hook 2 (HMGA2) in the patient, wherein an increased level of HMGA2 expression in the patient, relative to a known control level, identifies the patient as suitable for treating TNBC with said anti-TGFβ agent.

31. The anti-TGFβ agent for use of any one of claims 28 to 30, wherein the HMGA2 level of the patient is determined by analyzing a tissue sample from the patient.

32. The anti-TGFβ agent for use of claim 31, wherein the tissue sample is a biopsy sample, blood, serum, or plasma sample.

33. The anti-TGFβ agent for use of claim 31 or 32, wherein the level of HMGA2 is determined by immunochemistry or by RNA expression analysis.

34. The anti-TGFβ agent for use of any one of claims 28 to 33, wherein the increased HMGA2 expression has been determined via quantification of HMGA2 mRNA expression.

35. The anti-TGFβ agent for use of claim 34, wherein the quantification of HMGA2 mRNA expression is via PCR.

36. The anti-TGFβ agent for use of any one of claims 28 to 35, wherein the increased HMGA2 expression is at least 2.27-fold more than a known population mean HMGA2 expression among TNBC patients.

37. The anti-TGFβ agent for use of any one of claims 28 to 36, wherein the increased HMGA2 expression is at least 5-fold more than a known population mean HMGA2 expression among TNBC patients.

38. The anti-TGFβ agent for use of any one of claims 28 to 35, wherein the increased HMGA2 expression is at least 200% higher, at least 300% higher, at least 400% higher, at least 500% higher, at least 600% higher, at least 700% higher, at least 800% higher, at least 900% higher, at least 1000% higher, or more than the normal level of HMGA2 expression.

39. The anti-TGFβ agent for use of any one of claims 28 to 35, wherein the increased HMGA2 expression in the patient is at least 19- to 35-fold more than the HMGA2 expression in a patient who is non-responsive to a treatment with the anti-TGFβ agent.

40. The anti-TGFβ agent for use of any one of claims 28 to 33, wherein the increased HMGA2 expression has been determined by HMGA2 protein expression level.

41. The anti-TGFβ agent for use of claim 40, wherein the increased HMGA2 protein expression level has been determined via immunohistochemistry.

42. The anti-TGFβ agent for use of claim 41, wherein more than 1% tumor cells expressing HMGA2 protein in a tissue sample obtained from the TNBC patient determined the increased HMGA2 protein expression level.

43. The anti-TGFβ agent for use of any one of claims 28 to 42, wherein the anti-TGFβ agent is an anti-PD-L1/TGFβ Trap protein comprising a first polypeptide comprising: (a) at least a variable region of a heavy chain of an antibody that binds to human protein Programmed Death Figand 1 (PD-F1); and (b) human Transforming Growth Factor β Receptor II (TGFβRII), or a fragment thereof, capable of binding Transforming Growth Factor β (TGFβ), and a second polypeptide comprising at least a variable region of a light chain of an antibody that binds PD-F1; wherein the heavy chain of the first polypeptide and the light chain of the second polypeptide, when combined, form an antigen binding site that binds PD-F1.

44. The anti-TGFβ Trap agent for use of claim 43, wherein the first polypeptide comprises the amino acid sequences of SEQ ID NOs: 35, 36, and 37, and the second polypeptide comprises the amino acid sequences of SEQ ID NOs: 38, 39, and 40.

45. The anti-TGFβ Trap agent for use of claim 43 or 44, wherein the first polypeptide comprises the amino acid sequence of SEQ ID NO: 3, and the second polypeptide comprises the amino acid sequence of SEQ ID NO: 1.

46. The anti-TGFβ agent for use of any one of claims 43 to 45, wherein the dose of the anti-PD-L1/TGFβ Trap protein is 1200 mg to 3000 mg.

47. The anti-TGFβ agent for use of claim 46, wherein the dose of the anti-PD-L1/TGFβ Trap protein is 1200 mg.

48. The anti-TGFβ agent for use of claim 47, wherein the dose of the anti-PD-L1/TGFβ Trap protein is 1200 mg, administered once every two weeks.

49. The anti-TGFβ agent for use of any one of claims 43 to 46, wherein the dose of the anti-PD-L1/TGFβ Trap protein is 2100 mg to 2400 mg.

50. The anti-TGFβ agent for use of claim 49, wherein the anti-PD-L1/TGFβ Trap protein is administered once every three weeks.

51. The anti-TGFβ agent for use of claim 50, wherein the dose of the anti-PD-L1/TGFβ Trap protein is 2100 mg, administered once every three weeks.

52. The anti-TGFβ agent for use of claim 50, wherein the dose of the anti-PD-L1/TGFβ Trap protein is 2400 mg, administered once every three weeks.

53. The anti-TGFβ agent for use of any one of claims 43 to 46, wherein the dose of the anti-PD-L1/TGFβ Trap protein is 3000 mg, administered once every three weeks.

54. The anti-TGFβ agent for use of any one of claims 43 to 53, wherein the anti-PD-L1/TGFβ Trap protein is administered by intravenous administration.

55. The anti-TGFβ agent for use of claim 54, wherein the intravenous administration is performed with a prefilled bag, a prefilled pen, or a prefilled syringe comprising a formulation comprising the protein.

56. The anti-TGFβ agent for use of claim 55, wherein the bag is connected to a channel comprising a tube and/or a needle.


SEQUENCE LISTING (67 sequences)

SEQ ID NO: 1 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 2 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 607 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - DNA, 711 bp, Artificial Sequence
  Description of Artificial Sequence Synthetic polynucleotide
atgagggccctgctggctagactgctgctgtgcgtgctggtcgtgtccgacagcaagggccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctcatga

SEQ ID NO: 5 - DNA, 1887 bp, Artificial Sequence
  Description of Artificial Sequence Synthetic polynucleotide
atggaaacagacaccctgctgctgtgggtgctgctgctgtgggtgcccggctccacaggcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggtggcagcggtggcggtggctccggcggaggtggctccggaatccctccccacgtgcagaagtccgtgaacaacgacatgatcgtgaccgacaacaacggcgccgtgaagttccctcagctgtgcaagttctgcgacgtgaggttcagcacctgcgacaaccagaagtcctgcatgagcaactgcagcatcacaagcatctgcgagaagccccaggaggtgtgtgtggccgtgtggaggaagaacgacgaaaacatcaccctcgagaccgtgtgccatgaccccaagctgccctaccacgacttcatcctggaagacgccgcctcccccaagtgcatcatgaaggagaagaagaagcccggcgagaccttcttcatgtgcagctgcagcagcgacgagtgcaatgacaacatcatctttagcgaggagtacaacaccagcaaccccgactgataa

SEQ ID NO: 6 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 607 aa, Artificial Sequence
  Description of Artificial Sequence Synthetic polypeptide
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 9 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 10 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 21 aa, Artificial Sequence
  Description of Artificial Sequence Synthetic peptide
GGGGSGGGGSGGGGSGGGGSG

SEQ ID NO: 12 - protein, 118 aa, Artificial Sequence [sequence omitted]

SEQ ID NO: 13 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 118 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 15 - protein, 4 aa, Artificial Sequence
  Description of Artificial Sequence Synthetic peptide
QFNS

SEQ ID NO: 16 - protein, 4 aa, Artificial Sequence
  Description of Artificial Sequence Synthetic peptide
QAQS

SEQ ID NO: 17 - protein, 6 aa, Artificial Sequence
  Description of Artificial Sequence Synthetic peptide
PKSCDK

SEQ ID NO: 18 - protein, 6 aa, Artificial Sequence
  Description of Artificial Sequence Synthetic peptide
PKSSDK

SEQ ID NO: 19 - protein, 4 aa, Artificial Sequence
  Description of Artificial Sequence Synthetic peptide
LSLS

SEQ ID NO: 20 - protein, 4 aa, Artificial Sequence
  Description of Artificial Sequence Synthetic peptide
ATAT

SEQ ID NO: 21 - protein, 5 aa, Artificial Sequence
  Description of Artificial Sequence Synthetic peptide
  MOD_RES (1)..(1): Lys, Arg, Thr, Gln, Gly, Ala, Trp, Met, Ile or Ser
  MOD_RES (3)..(3): Val, Arg, Lys, Leu, Met or Ile
  MOD_RES (5)..(5): His, Thr, Asn, Gln, Ala, Val, Tyr, Trp, Phe or Met
XYXMX

SEQ ID NO: 22 - protein, 17 aa, Artificial Sequence
  Description of Artificial Sequence Synthetic peptide
  MOD_RES (8)..(8): Phe or Ile
  MOD_RES (14)..(14): Ser or Thr
SIYPSGGXTFYADXVKG

SEQ ID NO: 23 - protein, 11 aa, Artificial Sequence
  Description of Artificial Sequence Synthetic peptide
  MOD_RES (10)..(10): Glu or Asp
IKLGTVTTVXY

SEQ ID NO: 24 - protein, 30 aa, Artificial Sequence
  Description of Artificial Sequence Synthetic polypeptide
EVQLLESGGGLVQPGGSLRLSCAASGFTFS

SEQ ID NO: 25 - protein, 14 aa, Artificial Sequence
  Description of Artificial Sequence Synthetic peptide
WVRQAPGKGLEWVS

SEQ ID NO: 26 - protein, 32 aa, Artificial Sequence
  Description of Artificial Sequence Synthetic polypeptide
RFTISRDNSKNTLYLQMNSLRAEDTAVYYCAR

SEQ ID NO: 27 - protein, 11 aa, Artificial Sequence
  Description of Artificial Sequence Synthetic peptide
WGQGTLVTVSS

SEQ ID NO: 28 - protein, 14 aa, Artificial Sequence
  Description of Artificial Sequence Synthetic peptide
  MOD_RES (4)..(4): Asn or Ser
  MOD_RES (5)..(5): Thr, Arg or Ser
  MOD_RES (9)..(9): Ala or Gly
TGTXXDVGXYNYVS

SEQ ID NO: 29 - protein, 7 aa, Artificial Sequence
  Description of Artificial Sequence Synthetic peptide
  MOD_RES (1)..(1): Glu or Asp
  MOD_RES (3)..(3): Ile, Asn or Ser
  MOD_RES (4)..(4): Asp, His or Asn
XVXXRPS

SEQ ID NO: 30 - protein, 10 aa, Artificial Sequence
  Description of Artificial Sequence Synthetic peptide
  MOD_RES (3)..(3): Phe or Tyr
  MOD_RES (5)..(5): Asn or Ser
  MOD_RES (6)..(6): Arg, Thr or Ser
  MOD_RES (7)..(7): Gly or Ser
  MOD_RES (8)..(8): Ile or Thr
SSXTXXXXRV

SEQ ID NO: 31 - protein, 22 aa, Artificial Sequence
  Description of Artificial Sequence Synthetic peptide
QSALTQPASVSGSPGQSITISC

SEQ ID NO: 32 - protein, 15 aa, Artificial Sequence
  Description of Artificial Sequence Synthetic peptide
WYQQHPGKAPKLMIY

SEQ ID NO: 33 - protein, 32 aa, Artificial Sequence
  Description of Artificial Sequence Synthetic polypeptide
GVSNRFSGSKSGNTASLTISGLQAEDEADYYC

SEQ ID NO: 34 - protein, 10 aa, Artificial Sequence
  Description of Artificial Sequence Synthetic peptide
FGTGTKVTVL

SEQ ID NO: 35 - protein, 5 aa, Artificial Sequence
  Description of Artificial Sequence Synthetic peptide
SYIMM

SEQ ID NO: 36 - protein, 17 aa, Artificial Sequence
  Description of Artificial Sequence Synthetic peptide
SIYPSGGITFYADTVKG

SEQ ID NO: 37 - protein, 11 aa, Artificial Sequence
  Description of Artificial Sequence Synthetic peptide
IKLGTVTTVDY

SEQ ID NO: 38 - protein, 14 aa, Artificial Sequence
  Description of Artificial Sequence Synthetic peptide
TGTSSDVGGYNYVS

SEQ ID NO: 39 - protein, 7 aa, Artificial Sequence
  Description of Artificial Sequence Synthetic peptide
DVSNRPS

SEQ ID NO: 40 - protein, 10 aa, Artificial Sequence
  Description of Artificial Sequence Synthetic peptide
SSYTSSSTRV

SEQ ID NO: 41 - protein, 5 aa, Artificial Sequence
  Description of Artificial Sequence Synthetic peptide
MYMMM

SEQ ID NO: 42 - protein, 17 aa, Artificial Sequence
  Description of Artificial Sequence Synthetic peptide
SIYPSGGITFYADSVKG

SEQ ID NO: 43 - protein, 14 aa, Artificial Sequence
  Description of Artificial Sequence Synthetic peptide
TGTSSDVGAYNYVS

SEQ ID NO: 44 - protein, 119 aa, Artificial Sequence
  Description of Artificial Sequence Synthetic polypeptide
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVTVSS

SEQ ID NO: 45 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 46 - protein, 120 aa, Artificial Sequence
  Description of Artificial Sequence Synthetic polypeptide
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 47 - protein, 110 aa, Artificial Sequence
  Description of Artificial Sequence Synthetic polypeptide
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 48 - DNA, 1407 bp, Artificial Sequence
  Description of Artificial Sequence Synthetic polynucleotide from human Fab library
atggagttgcctgttaggctgttggtgctgatgttctggattcctgctagctccagcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 49 - DNA, 705 bp, Artificial Sequence
  Description of Artificial Sequence Synthetic polynucleotide from human Fab library
atggagttgcctgttaggctgttggtgctgatgttctggattcctgcttccttaagccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctca

SEQ ID NO: 50 - protein, 117 aa, Homo sapiens
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 51 - protein, 115 aa, Homo sapiens
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 52 - protein, 122 aa, Homo sapiens [sequence omitted]

SEQ ID NO: 53 - protein, 110 aa, Homo sapiens
LCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 54 - protein, 122 aa, Artificial Sequence
  Description of Artificial Sequence Synthetic polypeptide
VTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 55 - protein, 118 aa, Artificial Sequence
  Description of Artificial Sequence Synthetic polypeptide
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSS

SEQ ID NO: 56 - protein, 113 aa, Artificial Sequence
  Description of Artificial Sequence Synthetic polypeptide
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIK

SEQ ID NO: 57 - protein, 119 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSS

SEQ ID NO: 58 - protein, 111 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 59 - protein, 445 aa, Artificial Sequence
  Description of Artificial Sequence Synthetic polypeptide
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 220 aa, Artificial Sequence
  Description of Artificial Sequence Synthetic polypeptide
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 446 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGA

SEQ ID NO: 62 - protein, 218 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 4150 bp, Homo sapiens
cttgaatcttggggcaggaactcagaaaacttccagcccgggcagcgcgcgcttggtgcaagactcaggagctagcagcccgtccccctccgactctccggtgccgccgctgcctgctcccgccaccctaggaggcgcggtgccacccactactctgtcctctgcctgtgctccgtgcccgaccctatcccggcggagtctccccatcctcctttgctttccgactgcccaaggcactttcaatctcaatctcttctctctctctctctctctctctctctctctctctctctctctctctctctctctcgcagggtggggggaagaggaggaggaattctttccccgcctaacatttcaagggacacaattcactccaagtctcttccctttccaagccgcttccgaagtgctcccggtgcccgcaactcctgatcccaacccgcgagaggagcctctgcgacctcaaagcctctcttccttctccctcgcttccctcctcctcttgctacctccacctccaccgccacctccacctccggcacccacccaccgccgccgccgccaccggcagcgcctcctcctctcctcctcctcctcccctcttctctttttggcagccgctggacgtccggtgttgatggtggcagcggcggcagcctaagcaacagcagccctcgcagcccgccagctcgcgctcgccccgccggcgtccccagccctatcacctcatctcccgaaaggtgctgggcagctccggggcggtcgaggcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgccaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggactatattaatcaccttctttgtaatcccttcacagtcccaggtttagtgaaaaactgctgtaaacacaggggacacagcttaacaatgcaacttttaattactgttttcttttttcttaacctactaatagtttgttgatctgataagcaagagtgggcgggtgagaaaaaccgaattgggtttagtcaatcactgcactgcatgcaaacaagaaacgtgtcacacttgtgacgtcgggcattcatataggaagaacgcggtgtgtaacactgtgtacacctcaaataccaccccaacccactccctgtagtgaatcctctgtttagaacaccaaagataaggactagatactactttctctttttcgtataatcttgtagacacttacttgatgatttttaactttttatttctaaatgagacgaaatgctgatgtatcctttcattcagctaacaaactagaaaaggttatgttcatttttcaaaaagggaagtaagcaaacaaatattgccaactcttctatttatggatatcacacatatcagcaggagtaataaatttactcacagcacttgttttcaggacaacacttcattttcaggaaatctacttcctacagagccaaaatgccatttagcaataaataacacttgtcagcctcagagcatttaaggaaactagacaagtaaaattatcctctttgtaatttaatgaaaaggtacaacagaataatgcatgatgaactcacctaattatgaggtgggaggagcgaaatctaaatttcttttgctatagttatacatcaatttaaaaagcaaaaaaaaaaaaggggggggcaatctctctctgtgtctttctctctctctcttcctctccctctctcttttcattgtgtatcagtttccatgaaagacctgaataccacttacctcaaattaagcatatgtgttacttcaagtaatacgttttgacataagatggttgaccaaggtgcttttcttcggcttgagttcaccatctcttcattcaaactgcacttttagccagagatgcaatatatccccactactcaatactacctctgaatgttacaacgaatttacagtctagtacttattacatgctgctatacacaagcaatgcaagaaaaaaacttactgggtaggtgattctaatcatctgcagttctttttgtacacttaattacagttaaagaagcaatctccttactgtgtttcagcatgactatgtatttttctatgtttttttaattaaaaatttttaaaatacttgtttcagcttctctgctagatttctacattaacttgaaaattttttaaccaagtcgctcctaggttcttaaggataattttcctcaatcacactacacatcacacaagatttgactgtaatatttaaatattaccctccaagtctgtacctcaaatgaattctttaaggagatggactaattgacttgcaaagacctacctccagacttcaaaaggaatgaacttgttacttgcagcattcatttgttttttcaatgtttgaaatagttcaaactgcagctaaccctagtcaaaactatttttgtaaaagacatttgatagaaaggaacacgtttttacatacttttgcaaaataagtaaataataaataaaataaaagccaaccttcaaagaaacttgaagctttgtaggtgagatgcaacaagccctgcttttgcataatgcaatcaaaaatatgtgtttttaagattagttgaatataagaaaatgcttgacaaatattttcatgtattttacacaaatgtgatttttgtaatatgtctcaaccagatttattttaaacgcttcttatgtagagtttttatgcctttctctcctagtgagtgtgctgactttttaacatggtattatcaactgggccaggaggtagtttctcatgacggcttttgtcagtatggcttttagtactgaagccaaatgaaactcaaaaccatctctcttccagctgcttcagggaggtagtttcaaaggccacatacctctctgagactggcagatcgctcactgttgtgaatcaccaaaggagctatggagagaattaaaactcaacattactgttaactgtgcgttaaataagcaaataaacagtggctcataaaaataaaagtcgcattccatatctttggatgggccttttagaaacctcattggccagctcataaaatggaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctacataagactggacatttaacttttctaccatttctgcaagttaggtatgtttgcaggagaaaagtatcaagacgtttaactgcagttgactttctccctgttcctttgagtgtcttctaactttattctttgttctttatgtagaattgctgtctatgattgtactttgaatcgcttgcttgttgaaaatatttctctagtgtattatcactgtctgttctgcacaataaacataacagcctctgtgatccccatgtgttttgattcctgctctttgttacagttccattaaatgagtaataaagtttggtcaaaacagaaaaaaaaaaa

SEQ ID NO: 64 - DNA, 4900 bp, Homo sapiens
ccttgccaagtaacagctttgctgtccaacatcgtgtgctgcttcgcgagaaagtcacattcggaccctttggctagattgcttattcatagggcttcttgactaaagcccttggagcactgggtttttcttgaagtatatgatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaagcaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtattttt

SEQ ID NO: 65 - DNA, 386 bp, Artificial Sequence
  Description of Artificial Sequence Synthetic polynucleotide
gcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggtcaatgttgccttgcctgggaaggaccacccgggcaatcttatatatctactgttctctaaa

SEQ ID NO: 66 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 67 - protein, 1116 aa, Homo sapiens
MILDEFYNVKFCIDASQPDVGSWLKYIRFAGCYDQHNLVACQINDQIFYRVVADIAPGEELLLFMKSEDYPHETMAPDIHEERQYRCEDCDQLFESKAELADHQKFPCSTPHSAFSMVEEDFQQKLESENDLQEIHTIQECKECDQVFPDLQSLEKHMLSHTEEREYKCDQCPKAFNWKSNLIRHQMSHDSGKHYECENCAKQVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGANRHPAGLTFPTAPGFSFSFPGLFPSGLYHRPPLIPASSPVKGLSSTEQTNKSQSPLMTHPQILPATQDILKALSKHPSVGDNKPVELQPERSSEERPFEKISDQSESSDLDDVSTPSGSDLETTSGSDLESDIESDKEKFKENGKMFKDKVSPLQNLASINNKKEYSNHSIFSPSLEEQTAVSGAVNDSIKAIASIAEKYFGSTGLVGLQDKKVGALPYPSMFPLPFFPAFSQSMYPFPDRDLRSLPLKMEPQSPGEVKKLQKGSSESPFDLTTKRKDEKPLTPVPSKPPVTPATSQDQPLDLSMGSRSRASGTKLTEPRKNHVFGGKKGSNVESRPASDGSLQHARPTPFFMDPIYRVEKRKLTDPLEALKEKYLRPSPGFLFHPQFQLPDQRTWMSAIENMAEKLESFSALKPEASELLQSVPSMFNFRAPPNALPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESTGAILDDKEDAYFTEIRNFIGNSNHGSQSPRNVEERMNGSHFKDEKALVTSQNSDLLDDEEVEDEVLLDEEDEDNDITGKTGKEPVTSNLHEGNPEDDYEETSALEMSCKTSPVRYKEEEYKSGLSALDHIRHFTDSLKMRKMEDNQYSEAELSSFSTSHVPEELKQPLHRKSKSQAYAMMLSLSDKESLHSTSHSSSNVWHSMARAAAESSAIQSISHV